(19) United States
(12) Patent Application Publication
(10) Pub. No.: US 2021/0222168 A1
(43) Pub. Date: Jul. 22, 2021

(54) METHODS FOR REACTIVATING GENES ON THE INACTIVE X CHROMOSOME

(71) Applicant: THE GENERAL HOSPITAL CORPORATION, Boston, MA (US)

(72) Inventors: Jeannie T. Lee, Boston, MA (US); Lieselot Carrette, Beernem (BE)

(21) Appl. No.: 16/769,078

(22) PCT Filed: Dec. 3, 2018
(86) PCT No.: PCT/US2018/063690

§ 371 Date: Jun. 2, 2020

Related U.S. Application Data
(60) Provisional application No. 62/594,378, filed on Dec. 4, 2017.

(51) Int. Cl. C12N 15/113; A61K 31/7088; A61K 31/712

(52) U.S. Cl. CPC C12N 15/113; A61K 31/7088; A61K 31/712; C12N 2310/321; C12N 2320/34; C12N 2310/346; C12N 2310/315; C12N 2310/322; C12N 2310/341; C12N 2320/31; C12N 2310/3231; C12N 2310/113; C12N 2310/14

(57) ABSTRACT

Methods for reactivating genes on the inactive X chromosome that include administering an inhibitor of XIST RNA and an inhibitor of an Xist-interacting protein, e.g., a chromatin-modifying protein, e.g., a small molecule or an inhibitory nucleic acid (such as a small inhibitory RNA (siRNAs) or antisense oligonucleotide (ASO)) that targets XIST RNA and/or a gene encoding an Xist-interacting protein, e.g., a chromatin-modifying protein.

### CLAIM OF PRIORITY

This application claims the benefit of U.S. Provisional Patent Application Ser. No. 62/594,378, filed on Dec. 4, 2017. The entire contents of the foregoing are hereby incorporated by reference.

### FEDERALLY SPONSORED RESEARCH OR DEVELOPMENT

This invention was made with Government support under Grant No. DA38695 awarded by the National Institutes of Health. The Government has certain rights in the invention.

### TECHNICAL FIELD

This invention relates to compositions of one or more inhibitors of Xist RNA and inhibitors of Xist-interacting proteins. Also described are methods of using said compositions to activate expression of one or more alleles in a cell—e.g., an inactive X-linked allele, an epigenetically silenced allele, or a hypomorphic allele. For example, described herein are methods for reactivating genes on the inactive X chromosome that include administering both of an inhibitor of Xist RNA (e.g., an antisense oligonucleotide (ASO), e.g., locked nucleic acid (LNA), that targets Xist RNA), and an inhibitor of Xist-interacting protein, e.g., a chromatin-modifying protein, e.g., a small molecule.

### BACKGROUND

Diseases caused by a mutation on the mammalian X-chromosome affect males and females very differently as males have only one X chromosome and females have two. Female X-chromosomes are, however, subject to a dosage compensation mechanism in which one X-chromosomes is inactivated and is termed the inactive X (Xi), while the other X chromosome is spared inactivation and termed the active X (Xa). Because of “X-chromosome inactivation” (XCI), the female mammal is a mosaic of cells that expresses either the maternal or paternal X-chromosome (Disteche C M. Dosage compensation of the sex chromosomes. Annu Rev Genet. 2012; 46:537-560; Maduro C et al. Fitting the puzzle pieces: The bigger picture of XCI. Trends Biochem Sci. 2016; 41:138-147; Lee J T. Gracefully ageing at 50, X-chromosome inactivation becomes a paradigm for RNA and chromatin control. Nat Rev Mol Cell Biol. 2011; 12:815-826). Thus, heterozygous X-linked mutations would affect approximately half of her somatic cells. For gene products with a non-cell-autonomous function, healthy cells can usually compensate for those expressing the mutation (e.g., Factor VIII for hemophilia). With mutations in gene products that fulfill a critical role within the cells that produce them on the other hand, deficits in just half of the body's somatic cells can result in a severe disorder. One well-known example is Rett Syndrome (RTT), a human neurodevelopmental disorder caused by a mutation in the methyl-CpG-binding protein 2 (MECP2), a chromatin-associated gene product that is crucial for neuronal development (Lyst M J et al. Rett syndrome: A complex disorder with simple roots. Nat Rev Genet. 2015; 16:261-275). Whereas males do not survive, females are typically born and remain symptom free until the first or second year of life. Then, symptoms arise that include motor abnormalities, severe seizures, absent speech, and autism (Katz D M et al. Rett syndrome: Crossing the threshold to clinical translation. Trends Neurosci. 2016; 39:100-113). To date, no disease-specific therapy is available for this disorder that affects 1 in 10,000 girls throughout the world.

Notably, females carry a potential cure within their own cells. Every affected cell harbors a normal but dormant copy of MECP2 on the inactive X (Xi) chromosome, which may, in principle, be reactivated to alleviate disease burden. Intriguingly, in male RTT mouse models, restoring normal Mecp2 expression can reverse disease after the onset of symptoms (Giacometti E et al. Partial rescue of MeCP2 deficiency by postnatal activation of MeCP2. Proc Natl Acad Sci USA. 2007; 104:1931-1936; Guy J et al. Reversal of neurological defects in a mouse model of Rett syndrome. Science. 2007; 315:1143-1147). There are, however, two obstacles to an Xi-reactivation strategy. First, sex chromosomal dosage compensation is known to be important throughout development and life: perturbing XCI by a germline deletion of the master regulator Xist resulted in inviable female embryos (Marahrens Y et al. Xist-deficient mice are defective in dosage compensation but not spermatogenesis. Genes Dev. 1997; 11:156-166); an epiblast-specific deletion of Xist caused severely reduced female fitness (Yang L et al. Female mice lacking Xist RNA show partial dosage compensation and survive to term. Genes Dev. 2016; 30:1747-1760); and a conditional deletion of Xist in blood caused fully penetrant hematologic cancers (Yildirim E et al. Xist RNA is a potent suppressor of hematologic cancer in mice. Cell. 2013; 152:727-742). Perturbing dosage balance via Xi-reactivation could therefore have untoward physiological consequences. On the other hand, loss of Xist and partial reactivation occurs naturally in lymphocytes (Wang J et al. Unusual maintenance of X chromosome inactivation predisposes female lymphocytes for increased expression from the inactive X. Proc Natl Acad Sci USA. 2016; 113:E2029-E2038) and Xi-reactivation may therefore be tolerated in vivo under controlled circumstances. A second challenge is that the Xi has been difficult to reactivate via pharmacological means due to multiple parallel mechanisms of epigenetic silencing (Disteche C M. Dosage compensation of the sex chromosomes. Annu Rev Genet. 2012; 46:537-560; Csankovszki G et al. Synergism of Xist RNA, DNA methylation, and histone hypoacetylation in maintaining X chromosome inactivation. J Cell Biol. 2001; 153:773-784). Progress has been made in recent years, however. Several siRNA screens identified several factors regulating Xi stability, but no overlap of candidates was observed between them (Bhatnagar S et al. Genetic and pharmacological reactivation of the mammalian inactive X chromosome. Proc Natl Acad Sci USA. 2014; 111:12591-12598; Chan K M et al. Diverse factors are involved in maintaining X chromosome inactivation. Proc Natl Acad Sci USA. 2011; 108:16699-16704), perhaps because the screens were not saturating. Others have identified the TGF-β pathway (Sripathy S et al. Screen for reactivation of MeCP2 on the inactive X chromosome identifies the BMP/TGF-β superfamily as a regulator of XIST expression. Proc Natl Acad Sci USA. 2017; 114:1619-1624), a synergism between Aurora kinase and DNA methylation in a primed small molecule screen (Lessing D et al. A high-throughput small molecule screen identifies synergism between DNA methylation and Aurora kinase pathways for X reactivation. Proc Natl Acad Sci USA. 2016; 113:14366-14371), as well as a synergism between a ribonucleotide reductase subunit (RRM2) and 5-aza-2′-deoxycytidine (Minkovsky A et al. A high-throughput screen of inactive X chromosome reactivation identifies the enhancement of DNA demethylation by 5-aza-2′-dC upon inhibition of ribonucleotide reductase. Epigenetics Chromatin. 2015; 8:42). In a more direct approach, an Xist RNA proteomic screen identified more than a hundred interacting proteins and demonstrated that de-repression of the Xi could be achieved robustly only when 2-3 interactors were targeted simultaneously (Minajigi A et al. A comprehensive Xist interactome reveals cohesin repulsion and an RNA-directed chromosome conformation. Science. 2015; 349:aab2276-12).

### SUMMARY

In all studies to date, MECP2 restoration has been extremely limited (<<1% of normal levels). Described herein is a new mixed modality approach including an antisense oligonucleotide (ASO) against Xist RNA and an inhibitor of an Xist-interacting protein, the combination of which confers reactivation of MECP2 gene expression on the Xi.

Provided herein are methods and compositions for reactivating genes on the inactive or active X chromosome.

Provided herein are compositions comprising an inhibitor of Xist RNA and an inhibitor of an Xist-interacting protein.

Also provided herein are methods for activating an inactive X-linked allele in a cell, preferably a cell of a female heterozygous subject or a male hemizygous subject. The methods include administering to the cell an inhibitor of Xist RNA and an inhibitor of an Xist-interacting protein. As used herein, “an inhibitor of an Xist-interacting protein” can include one or more inhibitors, e.g., one or more small molecules or inhibitory nucleic acids. As used herein, “an inhibitor of Xist RNA” can include one or more inhibitors, e.g., one or more small molecules or inhibitory nucleic acids, e.g., an antisense oligonucleotide (ASO), e.g., locked nucleic acid (LNA), that target XIST RNA or a gene encoding XIST RNA.

In addition, provided herein are methods for activating an epigenetically silenced or hypomorphic allele on the active X-chromosome, e.g., FMRI, in a cell, e.g., in a cell of a male or female heterozygous subject. The methods include administering to the cell an inhibitor of Xist RNA and an inhibitor of an Xist-interacting protein.

Also provided here are an inhibitor of Xist and an inhibitor of an Xist-interacting protein, for use in activating an inactive X-linked allele in a cell, preferably a cell of a female heterozygous subject, preferably wherein the inactive X-linked allele is associated with an X-linked disorder.

Also provided here are an inhibitor of Xist RNA and an inhibitor of an Xist-interacting protein, for use in activating an epigenetically silenced or hypomorphic allele on the active X chromosome in a cell, either in a female heterozygous or male hemizygous subject, preferably wherein the active X-linked allele is associated with an X-linked disorder.

Also provided here are an inhibitor of Xist RNA and an inhibitor of an Xist-interacting protein, for use in treating an X-linked disorder in a female heterozygous or male hemizygous subject.

In some embodiments of the methods or compositions described herein, the inhibitor of Xist RNA is an inhibitory nucleic acid that targets the Xist lncRNA, e.g., e.g., an antisense oligonucleotide (ASO), e.g., locked nucleic acid (LNA), or that targets a gene encoding XIST.

In some embodiments of the methods or compositions described herein, the inhibitor of an Xist-interacting protein inhibits a protein described herein, e.g., shown in Table 1 or in tables 5 or 6 of WO2016164463, e.g., SMC1a; SMC3; WAPL, RAD21; KIF4; PDS5a/b; CTCF; TOP1; TOP2a; TOP2b; SMARCA4 (BRG1); SMARCA5; SMARCC1; SMARCC2; SMARCB1; CBX7; RING1a/b (PRC1); PRC2 (EZH2, SUZ12, RBBP7, RBBP4, EED); AURKB; SPEN/MINT/SHARP; DNMT1; SmcHD1; CTCF; MYEF2; ELAV1; SUN2; Lamin-B Receptor (LBR); LAP; hnRPU/SAF-A; hnRPK; hnRPC; PTBP2; RALY; MATRIN3; MacroH2A; and ATRX. In some embodiments, the Xist-interacting protein is not DNMT and/or is not topoisomerase, e.g., the inhibitor is not etoposide or 5′-azacytidine (aza),

In some embodiments of the methods or compositions described herein, the inhibitor of an Xist-interacting protein is a small molecule inhibitor or an inhibitory nucleic acid that targets a gene encoding the Xist-interacting protein.

In some embodiments of the methods or compositions described herein, the inactive X-linked allele is associated with an X-linked disorder, and the inhibitor of Xist RNA and inhibitor of Xist-interacting protein are administered in a therapeutically effective amount.

In some embodiments of the methods or compositions described herein, the active X-linked allele is associated with an X-linked disorder, and the inhibitor of Xist RNA and inhibitor of Xist-interacting protein are administered in a therapeutically effective amount.

In some embodiments of the methods described herein, the cell is in a living subject.

In some embodiments of the methods or compositions described herein, the inhibitory nucleic acid does not comprise three or more consecutive guanosine nucleotides or does not comprise four or more consecutive guanosine nucleotides.

In some embodiments of the methods or compositions described herein, the inhibitory nucleic acid is 8 to 30 nucleotides in length.

In some embodiments of the methods or compositions described herein, at least one nucleotide of the inhibitory nucleic acid is a nucleotide analogue.

In some embodiments of the methods or compositions described herein, at least one nucleotide of the inhibitory nucleic acid comprises a 2′ O-methyl, e.g., wherein each nucleotide of the inhibitory nucleic acid comprises a 2′ O-methyl.

In some embodiments of the methods or compositions described herein, the inhibitory nucleic acid comprises at least one ribonucleotide, at least one deoxyribonucleotide, or at least one bridged nucleotide.

In some embodiments of the methods or compositions described herein, the bridged nucleotide is a LNA nucleotide, a cEt nucleotide or a ENA modified nucleotide.

In some embodiments of the methods or compositions described herein, each nucleotide of the inhibitory nucleic acid is a LNA nucleotide.

In some embodiments of the methods or compositions described herein, one or more of the nucleotides of the inhibitory nucleic acid comprise 2′-fluoro-deoxyribonucleotides and/or 2′-O-methyl nucleotides.

In some embodiments of the methods or compositions described herein, one or more of the nucleotides of the inhibitory nucleic acid comprise one of both of ENA nucleotide analogues or LNA nucleotides.

In some embodiments of the methods or compositions described herein, the nucleotides of the inhibitory nucleic acid comprise comprising phosphorothioate internucleotide linkages between at least two nucleotides, or between all nucleotides.

In some embodiments of the methods or compositions described herein, the inhibitory nucleic acid is a gapmer or a mixmer.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a schematic representation showing the directionality and regions targeted by anti-sense oligonucleotides (ASOs) on the X chromosome.

FIG. 1B is a graph depicting luciferase assay results corrected counts per second (CCPS) normalized to the amount of cells of Xi-Mecp2-Luc MEFs transfected with 20 nM indicated ASO and treated with 0.5 uM 5-aza-2′-deoxycytidine (Aza) over 3 days.

FIG. 1C is a list of the small molecule inhibitors tested and their protein targets.

FIG. 1D is a graph depicting luciferase assay results CCPS measured at 3 days normalized to the amount of cells of Xi-Mecp2-Luc MEFs either untreated or transfected with 20 nM XIST gapmer 1 ASO (labeled as ‘Xist ASO’) and/or incubated with the indicated small molecule inhibitors.

FIG. 2A is a schematic representation of the Xist locus with the LoxP sites of the conditional deletion allele denoted by triangles, regions targeted by ASOs denoted by vertical bars, and conserved Xist repeat elements A-E labeled.

FIG. 2B is a graph depicting the average fold change in Xist RNA expression normalized to GAPDH expression in cells at 3 days transfected with negative control ASO (scrambled sequence, Scr) or transfected with XIST gapmer 1 ASO (herein referred to as ‘Xist ASO’) as compared to untreated cells; error bars represent the standard error of the mean (SEM).

FIG. 2C are representations of bright field microscope images of Xi-Mecp2-Luc MEF cells transfected with 20 nM ‘Xist ASO’ or negative control ASO and further treated with the indicated concentrations of Aza.

FIGS. 2D-2E are graphs depicting luciferase assay results of Xi-Mecp2-Luc MEFs transfected either with negative control ASO (scrambled sequence, Scr) or with 20 nM ‘XIST ASO’ and treated with 0.5 uM Aza over 3 or 5 days; p values determined by with Mann-Whitney U test (2-sided); error bars represent the SEM.

FIG. 3A is a schematic representation of the bioinformatics pipeline used to perform allele-specific analysis of RNA-sequencing (RNA-seq) data.

FIG. 3B are plots of normalized RNA sequencing reads aligning to the Xist gene locus in MEFs transfected with 20 nM ASOs and treated with 0.5 μM Aza for 3 days as compared to cells transfected with negative control ASO (scrambled sequence Scr) only; the scales (brackets) are set equal across treatments.

FIG. 3C is a cumulative distribution plot of percentage Xi expression ([number of mus reads/number of (mus+cas)] reads×100%) for MEF cells after each indicated 3 day treatment; p values were determined by Wilcoxon rank sum test (paired, one-sided); data from a single biological replicates is shown.

FIG. 3D is a heatmap generated with hierarchical clustering of Xi (mus) expression of X-inactivated genes in each indicated sample.

FIG. 3E is a scatterplot showing percentage Xi expression ([number of mus reads/number of (mus+cas)] reads×100%) for X-linked genes in cells treated with Aza and either transfected with locked nucleic acid (LNA) targeting XIST (labeled as XIST LNA) or with negative control ASO (scrambled sequence, Scr).

FIG. 3F are plots of normalized RNA-sequencing reads aligning to select reactivated genes in MEFs treated with 0.5 μM Aza and transfected with 20 nM ASOs for 3 days; the adjusted scales (brackets) set for Xi (mus: 0-0.25), or Xa (cas: 0-1), are shown within each gene.

FIG. 4A is a schematic representation of mating schemes to obtain F1 heterozygous and F2 homozygous female mice with brain-specific Xist deletion.

FIG. 4B are graphs depicting relative Xist expression normalized to GAPDH expression in brain and liver of F1 and F2 males and females; error bars represent the SEM.

FIG. 4C are representations of fluorescence microscopy images of DAPI and Xist RNA FISH stained brain and liver cells taken from indicated mice at age 530 days.

FIG. 4D is a graph depicting Kaplan-Maier survival curves for mice with specified genotypes.

FIG. 4E is a graph depicting rotarod analysis of one-year old female F1 mice with indicated genotypes and sample sizes; p values were determined by 2-sided T Student test with equal variance; error bars represent the SEM.

FIG. 4F is a graph depicting open field test results, specifically the ratio of the distance traveled in the center (measure of fear) to the total distance traveled (measure of activity) by 3-month old females of the four different genotypes; p values were determined by with Mann-Whitney U test (2-sided); error bars represent the SEM.

FIG. 4G-4H are cumulative distribution plots of fold-change in X-linked and autosomal gene expression comparing F2 homozygous female brain with Xist2lox/+control at 91 and 7 weeks (G and H, respectively); p values were determined by the Wilcoxon rank sum test (unpaired, one-sided) (n=1).

FIG. 5A are cumulative distribution plots of fold-change in X-linked and autosomal gene expression comparing control mice and test mice administered Aza three times over one week intraperitoneally at age 5 weeks; p values were determined by the Wilcoxon rank sum test (unpaired, one-sided) (n=1).

FIG. 5B is a graph depicting Kaplan-Maier survival curves for mice with specified genotypes; all mice survived to 1 year of age.

FIG. 5C is a graph depicting the weight of indicated mice before and after treatment; there was no statistically significant differences between the weights as determined by either one way ANOVA test or Brown-Forsythe test.

FIGS. 6A-6B are graphs depicting raw data from the luciferase assay of Mecp2-Luc MEFs treated with 0.5 uM Aza and transfected with different ASOs at 20 nM (Left) or transfected with ‘Xist ASO’ at 20 nM and treated with different small molecule inhibitors (Right) for 3 days; (A) depicts results as corrected counts per second (CCPS); (B) depicts results as number of cells harvested from one well of a 12-well plate.

FIG. 6C is a graph depicting average fold change of luciferase expression in treated Xi clones compared to Xa clone as determined qPCR in cells that were transfected by indicated ASO (negative control ASO or ‘XIST ASO’) at 20 nM and/or further treated by incubation with 0.5 μM Aza; error bars represent SEM for three biological replicates.

FIG. 7A is a graph depicting fold-change of Xist RNA expression normalized to GAPDH expression in MEFs transfected with ‘Xist ASO’ (labeled as XIST ASO 1) or transfected with Xist ASO 2, or transfected with XIST ASO 3 as compared to MEFs transfected with negative control ASO (scrambled sequence, Scr; labeled as control).

FIGS. 7B-7D are graphs depicting luciferase assay results of Xi-Mecp2-Luc MEFs treated with 0.5 μM Aza and transfected with 20 nM of indicated ASO compared to untreated Xi-Mecp2-Luc MEFs and compared to Xa-Mecp2-Luc MEFs; (B) depicts results as corrected counts per second (CCPS); (C) depicts results as number of cells harvested from one well of a 12-well plate; (D) depicts results as relative CCPS per number of cells; error bars represent SEM for three biological replicates.

FIG. 8A are representations of fluorescence microscopy images of DAPI and Xist RNA FISH stained brain and liver cells taken from indicated mice.

FIG. 8B is a graph depicting percentage of brain and liver cells zero, one, or two Xist RNA clouds as determined by RNA FISH.

FIG. 8C is a photo showing two representative female F1 littermates; Xist4/+(Left); Xist2lox/+(Right).

FIG. 8D is a graph depicting weights of F1 generations after one year with overlaid box and whisker plot; triangle denotes mean weight.

FIG. 9A is a graph depicting open field test results, specifically the average ratio of the distance traveled in the center (measure of fear) to the total distance traveled (measure of activity) by one-year-old F1 females; p values were determined by the Mann-Whitney U test.

FIG. 9B are representative graphs depicting an open field test for a one-year-old F1 XistA/+ female (Left) and for an age-matched Xist2lox/+ female (Right).

FIGS. 9C-9E are graphs depicting number of selected behaviors exhibited by F1 Xist4/+ female mice or F1 Xist2lox/+ female mice during the elevated cross maze expressed in events (C), time in (D), and percentage (E). Error bars represent the standard error.

FIG. 9F is a graph depicting time spent in the center of the elevated cross maze by F1 Xist4/+ female mice or F1 Xist2lox/+ female mice.

FIG. 10 is a cartoon representation of the mating scheme to separate Nestin-Cre+/− genotype from the Xist2lox/+ genotype.

### DETAILED DESCRIPTION

The Xi is a reservoir of >1000 functional genes that could, in principle, be reactivated, by increasing gene expression, to treat disorders caused by mutations or altered epigenetic regulation on the Xa. In the present study, we set out to define a pharmacological approach for selective Xi-reactivation to restore expression of X-linked gene products. We focused on RTT and restoration of MECP2 gene expression, but our Xi-reactivation platform is agnostic to both the disease and the X-linked gene product. Any gene residing on the X-chromosome could be targeted in phenotypic, heterozygous females.

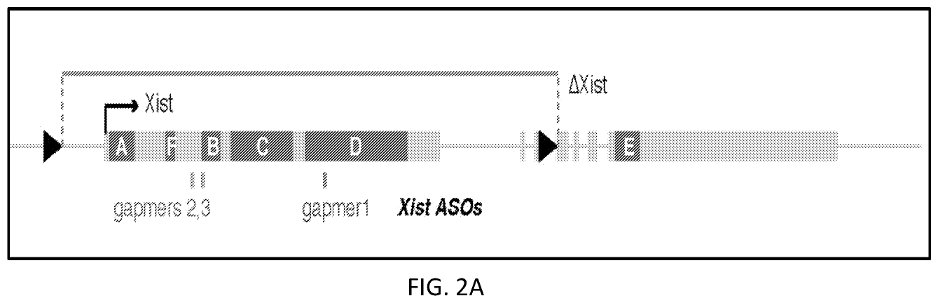

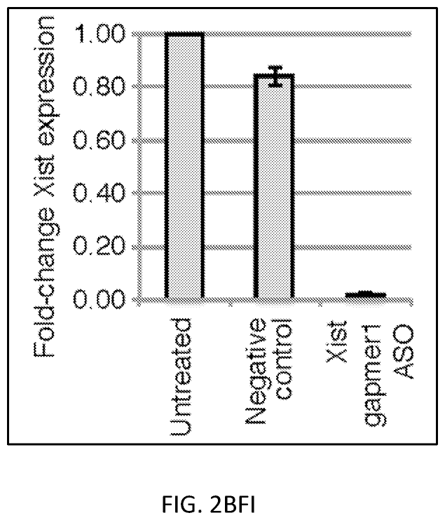

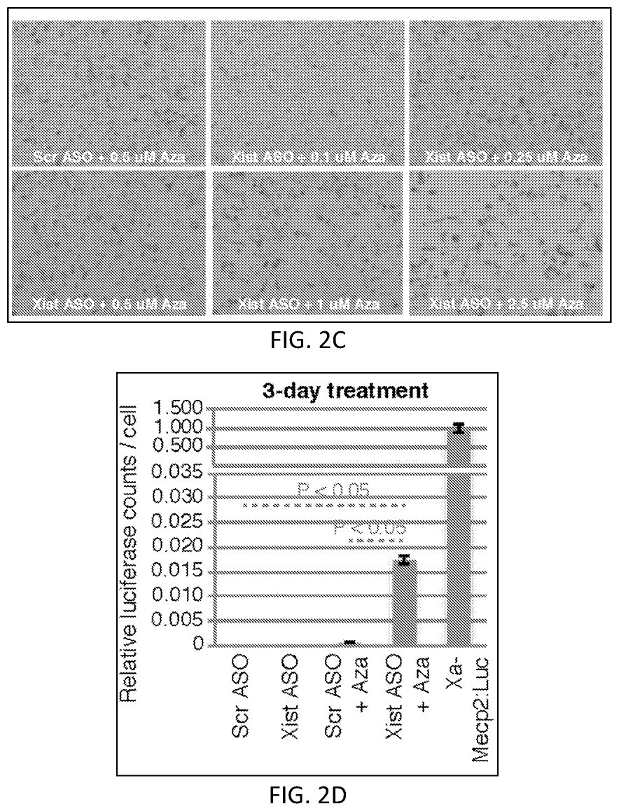

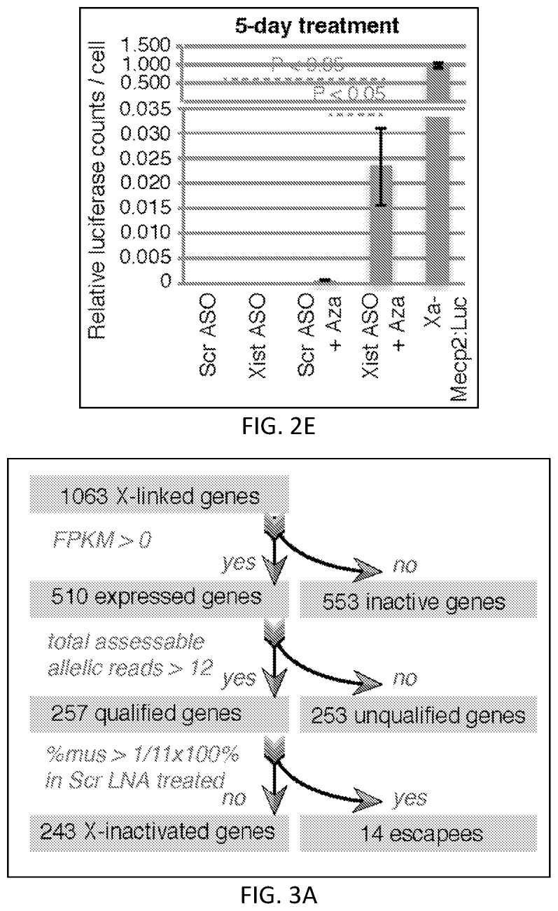

By targeting factors in the Xist interactome, we found that combining two drug modalities—an inhibitor of an Xist interacting protein, e.g., a small molecule inhibitor of EZH2 or AURKB, in combination with an anti-sense oligonucleotide (ASO) targeting Xist RNA—achieved an unprecedented level of Xi-reactivation, as measured by upregulated MECP2 gene expression and MECP2 protein. By targeting Xist RNA with an ASO and DNMT1 protein with decitabine (Aza), we observed a 2 to 5% upregulation—equivalent to a 12,000-30,000× increase in Xi-Mecp2 expression, which is considerably greater than the 600× upregulation observed in a previous screen (Lessing D et al. A high-throughput small molecule screen identifies synergism between DNA methylation and Aurora kinase pathways for X reactivation. Proc Natl Acad Sci USA. 2016; 113:14366-14371), and thus marks significant progress for the Xi-reactivation platform. Several other combinations also yielded a larger degree of reactivation than previously seen with any other compound; these combinations included Xist LNA in combination with EPZ6438 (an EZH2 inhibitor) and Xist LNA in combination with VX680 (an AURK inhibitor). In vivo data have suggested that even 5% of normal Mecp2 levels can have profound impact on survival and overall function, as a previous report showed slightly milder phenotype of the Mecp2-lox-stop-lox male mice, due to their “leaky” termination cassette that enabled read-through Mecp2 transcription (Guy J et al. Reversal of neurological defects in a mouse model of Rett syndrome. Science. 2007; 315:1143-1147). Thus, while the degree of MECP2 gene expression upregulation following treatment with Xist ASO in combination with Aza did not exceed 5% in the experiments disclosed herein, this degree of restoration could have significant phenotypic consequences in vivo. Moreover, because our treatment period was very brief—only 3-5 days—and the tolerable Aza concentrations in cell culture (0.5 μM; FIG. 2C) are still higher than concentrations typically used for mouse IP injections (Sales A J et al. Antidepressant-like effect induced by systemic and intra-hippocampal administration of DNA methylation inhibitors. Br J Pharmacol. 2011; 164:1711-1721), in vivo outcomes may be enhanced by applying a more concentrated dosage in future studies. Our present analysis cannot distinguish between high-level MECP2 reactivation from a few cells versus a low-level reactivation from a large percentage of cells. The two possibilities would have different physiological implications, but both are potentially relevant from a therapeutic standpoint, as MECP2 has been identified to have both cell-autonomous and non-cell-autonomous functions (Kishi and Macklis, (2010) Experimental Neurology 222(1):51-58).

ASO drugs are generally more specific and have the advantage that information on pharmacokinetics and toxicity studies for chemically similar ASOs is transferable and cumulative. Thus, ASOs may have a more favorable path to regulatory approval. Small molecules generally have lower selectivity and may face steeper hurdles in the approval process within the US Food and Drug Administration (FDA). By mixing modalities, our approach may potentially anticipate a more streamlined approach to FDA approval. We also note that Aza has already been FDA-approved for other disease indications (myelodysplastic syndrome and acute myeloid leukemia (Kishi N and Macklis J D. MeCP2 functions largely cell-autonomously, but also non-cell-autonomously, in neuronal maturation and dendritic arborization of cortical pyramidal neurons. Exp Neurol. 2010; 222:51-58). Furthermore, our present in vivo data indicate that Aza need not be given continuously or long term to observe an impact on Xi reactivation in the brain. Nor does Aza need to be injected into the target organ—e.g., the brain. Indeed, three short pulses delivered systemically at the beginning of the treatment period was sufficient to induce Xi reactivation after two weeks in the brain. Unlike LNA-based ASOs which have tissue half-lives of several weeks (Wahlestedt C et al. Potent and nontoxic antisense oligonucleotides containing locked nucleic acids. Proc Natl Acad Sci USA. 2000; 97:5633-5638), Aza is known to have a very short half-life (t_{1/2}<1 h in plasma) (Welch J S et al. TP53 and decitabine in acute myeloid leukemia and myelodysplastic syndromes. N Engl J Med. 2016; 375:2023-2036). However, once DNA is demethylated, the state may be stable (Kordasiewicz H B et al. Sustained therapeutic reversal of Huntington's disease by transient repression of huntingtin synthesis. Neuron. 2012; 74:1031-1044). Future work will determine the duration of the effect and whether periodic boosters of either Aza or ASO might be necessary to maintain the reactivation.

Finally, we are encouraged that partial Xi-reactivation in the brain does not cause apparent morbidity or mortality in the mouse. An important next step will be to test the combination drug in a Rett-specific disease model for phenotypic improvement. ASOs are well suited for the treatment of neurological diseases and their delivery may be targeted to the central nervous system through intracerebroventricular or intrathecal injection (Southwell A L et al. Antisense oligonucleotide therapeutics for inherited neurodegenerative diseases. Trends Mol Med. 2012; 18:634-643), which has been considered acceptable and safe for serious disease such as ALS (Karahoca M and Momparler R L. Pharmacokinetic and pharmacodynamic analysis of 5-aza-2′-deoxycytidine (decitabine; Aza) in the design of its dose-schedule for cancer therapy. Clin Epigenetics. 2013; 5:3). Another critical next step is the development of a better female mouse model, that recapitulates the RTT disease severity (Miller T M et al. An antisense oligonucleotide against SOD1 delivered intrathecally for patients with SOD1 familial amyotrophic lateral sclerosis: A phase 1, randomized, first-in-man study. Lancet Neurol. 2013; 12:435-442). Such a female mouse model will be essential to test our Xi reactivation platform in vivo.

### Methods of Reactivating Genes on the Inactive X Chromosome (Xi)

The present disclosure provides methods for reactivating genes on Xi by combining inhibitors for Xist-interacting factors (listed in Table 1). The methods include co-administering an inhibitor of an Xist-interacting factor (listed in Table 7), e.g., a small molecule, and a small inhibitory RNA (siRNAs) that targets Xist RNA. These methods can be used, e.g., to reactivate genes in single cells, e.g., isolated cells in culture, or in tissues, organs, or whole animals. In some embodiments, the methods are used to reactivate genes on Xi in a cell or subject that has an X-linked disease. X-reactivation can be achieved in various cell types, including proliferating fibroblasts and post-mitotic neurons.

The methods described herein can be also be used to specifically re-activate one or more genes on Xi, by co-administering an inhibitory nucleic acid targeting a suppressive RNA or genomic DNA at strong and/or moderate binding sites as described in WO 2012/065143, WO 2012/087983, and WO 2014/025887 or in U.S. Ser. No. 62/010,342 (which are incorporated herein in their entirety), to disrupt RNA-mediated silencing in cis on the inactive X-chromosome. The suppressive RNAs can be noncoding (e.g., long noncoding RNA (lncRNA)) or occasionally part of a coding mRNA; for simplicity, we will refer to them together as suppressive RNAs (supRNAs) henceforth. SupRNAs that mediate silencing of genes on the X chromosome are known in the art; see, e.g., WO 2012/065143, WO 2012/087983, WO 2014/025887 and U.S. Ser. No. 62/010,342, and inhibitory nucleic acids and small molecules targeting (e.g., complementary to) the sRNAs, or complementary or identical to a region within a strong or moderate binding site in the genome, e.g., as described in WO 2014/025887, can be used to modulate gene expression in a cell, e.g., a cancer cell, a stem cell, or other normal cell types for gene or epigenetic therapy. The nucleic acids targeting supRNAs that are used in the methods described herein are termed “inhibitory” (though they increase expression of the supRNA-repressed gene) because they inhibit the supRNAs-mediated repression of a specified gene. Without wishing to be bound to a particular theory, the nucleic acids targeting supRNAs may function either by directly binding to the supRNAs itself (e.g., an antisense oligo that is complementary to the supRNAs) or by binding to a strong or moderate binding site for an RNA-binding protein (e.g., PRC2—also termed an EZH2, SUZ12, and CTCF) in the genome, and in doing so, preventing binding of the RNA-binding protein complex and thus disrupting silencing in the region of the strong or moderate binding site. The inhibitory nucleic acids that bind to a strong or moderate RNA-binding protein binding site can bind to either strand of the DNA, but preferably bind to the same strand to which the supRNAs binds. See, e.g., WO 2012/065143, WO 2012/087983, WO 2014/025887 and U.S. Ser. No. 62/010,342.

The cells can be in vitro, including ex vivo, or in vivo (e.g., in a subject who has cancer, e.g., a tumor).

In some embodiments, the methods include introducing into the cell (or administering to a subject) an inhibitory ASO targeting XIST RNA and an inhibitor of an Xist-interacting protein, e.g., a chromatin-modifying protein, e.g., a small molecule inhibitor of an Xist-interacting protein.

In some embodiments, the methods include introducing into the cell (or administering to a subject) an inhibitory nucleic acid (e.g., targeting Xist RNA) that is modified in some way, e.g., an inhibitory nucleic acid that differs from the endogenous nucleic acids at least by including one or more modifications to the backbone or bases as described herein for inhibitory nucleic acids. Such modified nucleic acids are also within the scope of the present invention.

In some embodiments, the methods include introducing into the cell (or administering to a subject) an inhibitor of Xist RNA (e.g., a small inhibitory RNA (siRNA) or LNA that targets XIST) and an inhibitor of an Xist-interacting protein, e.g., AURKB or EZH2, e.g., a small molecule inhibitor, and optionally an inhibitory nucleic acid that specifically binds, or is complementary, to a strong or moderate binding site or a supRNA described in WO 2012/065143, WO 2012/087983, WO 2014/025887 and U.S. Ser. No. 62/010,342. A nucleic acid that binds “specifically” binds primarily to the target, i.e., to the target DNA, mRNA, or supRNA to inhibit regulatory function or binding of the DNA, mRNA, or supRNA, but does not substantially inhibit function of other non-target nucleic acids. The specificity of the nucleic acid interaction thus refers to its function (e.g., inhibiting gene expression) rather than its hybridization capacity. Inhibitory nucleic acids may exhibit nonspecific binding to other sites in the genome or other RNAs without interfering with binding of other regulatory proteins and without causing degradation of the non-specifically-bound RNA. Thus this nonspecific binding does not significantly affect function of other non-target RNAs and results in no significant adverse effects. These methods can be used to treat an X-linked condition in a subject by administering to the subject a composition or compositions (e.g., as described herein) comprising an inhibitor of Xist RNA and of an Xist-interacting protein, e.g., as listed in Table 1, e.g., a small molecule inhibitor, and optionally an inhibitory nucleic acid that specifically binds, or is complementary, to a strong or moderate binding site or a supRNA (e.g., as described in WO 2012/065143, WO 2012/087983, WO 2014/025887 and U.S. Ser. No. 62/010,342) that is associated with an X-linked disease gene. Examples of genes involved in X-linked diseases are shown in Table 2.

As used herein, treating includes “prophylactic treatment” which means reducing the incidence of or preventing (or reducing risk of) a sign or symptom of a disease in a patient at risk for the disease, and “therapeutic treatment”, which means reducing signs or symptoms of a disease, reducing progression of a disease, reducing severity of a disease, in a patient diagnosed with the disease.

In some embodiments, the methods described herein include administering a composition, e.g., a sterile composition, comprising an inhibitory nucleic acid that is complementary to Xist or a gene encoding Xist RNA, and an inhibitor of an Xist-interacting protein, e.g., as listed in Table 1, and optionally an inhibitory nucleic acid that is complementary to a supRNA as known in the art, e.g., as described in WO 2012/065143, WO 2012/087983, and/or WO 2014/025887. Inhibitory nucleic acids for use in practicing the methods described herein can be an antisense or small interfering RNA, including but not limited to an shRNA or siRNA. In some embodiments, the inhibitory nucleic acid is a modified nucleic acid polymer (e.g., a locked nucleic acid (LNA) molecule).

Inhibitory nucleic acids have been employed as therapeutic moieties in the treatment of disease states in animals, including humans. Inhibitory nucleic acids can be useful therapeutic modalities that can be configured to be useful in treatment regimens for the treatment of cells, tissues and animals, especially humans.

For therapeutics, an animal, preferably a human, who has an X-linked disorder is treated by administering an inhibitor of XIST RNA and an inhibitor of an Xist-interacting protein, e.g., as listed in Table 1, e.g., a small molecule inhibitor or an inhibitory nucleic acid such as a small inhibitory RNA (siRNA) or LNA that targets a gene encoding Xist RNA and/or an Xist-interacting protein, and optionally an inhibitory nucleic acid that is complementary to a supRNA.

### Inhibitor of XIST RNA

The methods include administering an inhibitor of an XIST RNA itself, e.g., an inhibitory nucleic acid targeting XIST RNA. Although in typical usage XIST refers to the human sequence and Xist to the mouse sequence, in the present application the terms are used interchangeably. The human XIST sequence is available in the ensemble database at ENSG00000229807; it is present on Chromosome X at 73,820,651-73,852,753 reverse strand (Human GRCh38.p2). The full sequence is shown in SEQ ID NO:66; XIST exons correspond to 601-11972 (exon 1); 15851-15914 (exon 2); 19593-20116 (exon 3); 21957-21984 (exon 4); 22080-22288 (exon 5); and 23887-33304 (exon 6). Alternatively, see NCBI Reference Sequence: NR_001564.2, Homo sapiens X inactive specific transcript (non-protein coding) (XIST), long non-coding RNA, wherein the exons correspond to 1-11372, 11373-11436, 11437-11573, 11574-11782, 11783-11946, and 11947-19280. The inhibitory nucleic acid targeting XIST RNA can be any inhibitory nucleic acid as described herein, and can include modifications described herein or known in the art. In some embodiments, the inhibitory nucleic acid is an antisense oligonucleotide (ASO) that targets a sequence in XIST RNA, e.g., a sequence within an XIST exon as shown in SEQ ID NO:66 or within the RNA sequence as set forth in NR_001564.2. In some embodiments, the inhibitory nucleic includes at least one locked nucleotide, e.g., is a locked nucleic acid (LNA).

### Xist-Interacting Proteins

The methods include administering an inhibitor of an Xist-interacting protein. Tables 5 and 6 of PCT/US2016/026218 (published as WO2016164463, which is incorporated by reference here in its entirety), and Table 1 herein, list Xist-interacting proteins, e.g., chromatin-modifying proteins, that can be targeted in the methods described herein. These inhibitors can include small molecules as well as inhibitory nucleic acids targeting the Xist-interacting protein.

Small molecule inhibitors of many of these Xist interactors are known in the art; see, e.g., Table 1, for examples. In addition, small molecule inhibitors of PRC1 or PRC2 components can be used; for example, inhibitors of EZH2 include UNC1999, E7438, N-[(4,6-dimethyl-2-oxo-1,2-dihydro-3-pyridinyl)methyl]-3-methyl-1-[(1S)-1-methylpropyl]-6-[6-(1-piperazinyl)-3-pyridinyl]-1H-indole-4-carboxamide, EPZ-6438 (N-((4,6-dimethyl-2-oxo-1,2-dihydropyridin-3-yl)methyl)-5-(ethyl(tetrahyd-ro-2H-pyran-4-yl)amino)-4-methyl-4′-(morpholinomethyl)-[1,1′-biphenyl]-3-carboxamide), GSK-126 ((S)-1-(sec-butyl)-N-(4,6-dimethyl-2-oxo-1,2-dihydropyridin-3-yl)methyl)-3-methyl-6-(6-(piperazin-1-yl)pyridin-3-yl)-1H-indole-4-carboxamide), GSK-343 (1-Isopropyl-N-((6-methyl-2-oxo-4-propyl-1,2-dihydropyridin-3-yl)-methyl)-6-(2-(4-methylpiperazin-1-yl)pyridine-4-yl)-1H-indazole-4-carboxam-ide), E11, 3-deazaneplanocin A (DNNep, 5R-(4-amino-1H-imidazo[4,5-c]pyridin-1-yl)-3-(hydroxymethyl)-3-cyclopente-ne-1S,2R-diol), isoliquiritigenin, and those provided in, for example, U.S. Publication Nos. 2009/0012031, 2009/0203010, 2010/0222420, 2011/0251216, 2011/0286990, 2012/0014962, 2012/0071418, 2013/0040906, US20140378470, US20140275081, US20140357688, and 2013/0195843; see also PCT/US2011/035336, PCT/US2011/035340, PCT/US2011/035344.

TABLE 1
Exemplary Xist-Interacting Proteins and Chromatin-Modifying Proteins

| Name of Xist- | |
|---|---|
| Interacting Protein | Small molecule inhibitor |
| WAPL | — |
| SMC1a | See above |
| SMC3 | See above |
| RAD21 | See above |
| KIF4 | — |
| PDS5a/b | See above |
| CTCF | 3-aminobenzamide |
| TOP1 | See above |
| TOP2a | See above |
| TOP2b | See above |
| SMARCA4 (BRG1) | PFI3 ((E)-1-(2-Hydroxyphenyl)-3-((1R,4R)-5-(pyridin-2-yl)-2,5- |
| | diazabicyclo[2.2.1]heptan-2-yl)prop-2-en-1-one); JQ1(+); AGN-PC- |
| | 0DAUWN |
| SMARCA5 | — |
| SMARCC1 | — |
| SMARCC2 | — |
| SMARCB1 | — |
| CBX2 | — |
| CBX4 | — |
| CBX5 | — |
| CBX6 | — |
| CBX7 | MS37452 |
| CBX8 | — |
| RINB1a | PRT4165 (2-pyridine-3-yl-methylene-indan-1,3-dione) |
| RING1b | — |
| AURKB | ZM447439, Hesperadin, VX-680/MK-0457 (4,6-diaminopyrimidine), |
| | AT9283, AZD1152, AKI-001, PHA-680632, VE-465, JNJ-7706621, |
| | CCT129202, MLN8237, ENMD-2076, MK-5108, PHA-739358, |
| | CYC116, SNS-314, R763, PF-03814375, GSK1070916, AMG-900 |
| | (see Kollareddy et al., Invest New Drugs. 2012 December; 30(6): 2411- |
| | 2432) |
| SPEN/MINT/SHARP | MG132 |
| DNMT1 | See, e.g., WO2016164463 |
| SmcHD1 | — |
| CTCF | — |
| MYEF2 | — |
| ELAVL1 | — |
| SUN2 | mevinolin |
| Lamin-B Receptor | — |
| (LBR) | |
| LAP | bestatin |
| hnRPU/SAF-A | - DPQ |
| hnRPK | — |
| hnRPC | — |
| PTBP2 | — |
| RALY | — |
| MATRIN3 | plumbagin |
| MacroH2A | |
| ATRX | Berberine, Inhibitors of histone deacetylases (HDAC) such as |
| | trichostatin A (TSA), depsipeptide, vorinostat, |
| RYBP | — |
| YY1 | — |
| EZH2 | See above |
| SUZ12 | — |
| EED | Astemizole (inhibits EZH2-EED interaction) |
| RBBP7 | — |
| RBBP4 | — |
| JARID2 | — |

### Inhibitory Nucleic Acids

The methods and compositions described herein can include nucleic acids such as a small inhibitory RNA (siRNA) or LNA that targets (specifically binds, or is complementary to) XIST RNA or to a gene encoding XIST or an XIST-interacting protein, e.g., a chromatin-modifying protein, and optionally an inhibitory nucleic acid that targets a strong or moderate binding site or a supRNA described in WO 2012/065143, WO 2012/087983, WO 2014/025887 and U.S. Ser. No. 62/010,342. Inhibitory nucleic acids useful in the present methods and compositions include antisense oligonucleotides, ribozymes, external guide sequence (EGS) oligonucleotides, siRNA compounds, single- or double-stranded RNA interference (RNAi) compounds such as siRNA compounds, molecules comprising modified bases, locked nucleic acid (LNA) molecules, bridged nucleic acid (BNA) molecules, peptide nucleic acid (PNA) molecules, and other oligomeric compounds or oligonucleotide mimetics which hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof. See, e.g., U.S. Ser. No. 62/010,342, WO 2012/065143, WO 2012/087983, and WO 2014/025887. However, in some embodiments the inhibitory nucleic acid is not an miRNA, an stRNA, an shRNA, an siRNA, an RNAi, or a dsRNA.

| Human | | Human | | |
|---|---|---|---|---|
| Gene | Human | Gene | Gene | |
| ID | Protein | symbol | Synonyms | Accession numbers |
| 2146 | EZH2 | EZH2 | KMT6; | XM_011515896; |
| | | | KMT6A; | XM_011515897; |
| | | | WVS; | XM_011515901; |
| | | | EZH2b; | NM_001203249; |
| | | | ENX-1; | XM_005249964; |
| | | | EZH1; | XM_011515884; |
| | | | ENX1; | XM_011515890; |
| | | | WVS2 | XM_011515894; |
| | | | | XM_011515899; |
| | | | | NM_004456; |
| | | | | XM_011515886; |
| | | | | XM_011515892; |
| | | | | XM_011515900; |
| | | | | NM_152998; |
| | | | | XM_011515888; |
| | | | | XM_011515889; |
| | | | | XM_011515902;; |
| | | | | NM_001203247; |
| | | | | NM_001203248; |
| | | | | XM_005249962; |
| | | | | XM_011515895; |
| | | | | XM_011515883; |
| | | | | XM_005249963; |
| | | | | XM_011515885; |
| | | | | XM_011515887; |
| | | | | XM_011515898; |
| | | | | XM_011515891; |
| | | | | XM_011515893 |
| 9212 | AURKB | AURKB | STK5; | XM_011524070; |
| | | | aurkb-sv2; | XR_934118; |
| | | | AurB; | NM_001256834; |
| | | | ARK2; | XM_011524071; |
| | | | PPP1R48; | XR_934117; |
| | | | aurkb-sv1; | NM_001284526; |
| | | | AIM-1; | NM_004217; |
| | | | AIK2; | XM_011524072 |
| | | | IPL1; | |
| | | | AIM1; | |
| | | | STK12; | |
| | | | STK-1; | |
| | | | ARK-2 | |

In some embodiments, the inhibitory nucleic acids are 10 to 50, 10 to 20, 10 to 25, 13 to 50, or 13 to 30 nucleotides in length. One having ordinary skill in the art will appreciate that this embodies inhibitory nucleic acids having complementary portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin. In some embodiments, the inhibitory nucleic acids are 15 nucleotides in length. In some embodiments, the inhibitory nucleic acids are 12 or 13 to 20, 25, or 30 nucleotides in length. One having ordinary skill in the art will appreciate that this embodies inhibitory nucleic acids having complementary portions of 12, 13, 14, 15, 16, 17, 18, 19, 20, 21, 22, 23, 24, 25, 26, 27, 28, 29 or 30 nucleotides in length, or any range therewithin (complementary portions refers to those portions of the inhibitory nucleic acids that are complementary to the target sequence).

The inhibitory nucleic acids useful in the present methods are sufficiently complementary to the target RNA, i.e., hybridize sufficiently well and with sufficient specificity, to give the desired effect. “Complementary” refers to the capacity for pairing, through hydrogen bonding, between two sequences comprising naturally or non-naturally occurring bases or analogs thereof. For example, if a base at one position of an inhibitory nucleic acid is capable of hydrogen bonding with a base at the corresponding position of a RNA, then the bases are considered to be complementary to each other at that position. 100% complementarity between inhibitory nucleic acid and target is not required for the inhibitory nucleic acid to sufficiently inhibit function of the target.

Routine methods can be used to design an inhibitory nucleic acid that binds to the target sequence with sufficient specificity. In some embodiments, the methods include using bioinformatics methods known in the art to identify regions of secondary structure, e.g., one, two, or more stem-loop structures, or pseudoknots, and selecting those regions to target with an inhibitory nucleic acid. For example, “gene walk” methods can be used to optimize the inhibitory activity of the nucleic acid; for example, a series of oligonucleotides of 10-30 nucleotides spanning the length of a target RNA can be prepared, followed by testing for activity. Optionally, gaps, e.g., of 5-10 nucleotides or more, can be left between the target sequences to reduce the number of oligonucleotides synthesized and tested. GC content is preferably between about 30-60%. Contiguous runs of three or more Gs or Cs should be avoided where possible (for example, it may not be possible with very short (e.g., about 9-10 nt) oligonucleotides).

In some embodiments, the inhibitory nucleic acid molecules can be designed to target a specific region of the RNA sequence. For example, a specific functional region can be targeted, e.g., a region comprising a known RNA localization motif (i.e., a region complementary to the target nucleic acid on which the RNA acts). Alternatively or in addition, highly conserved regions can be targeted, e.g., regions identified by aligning sequences from disparate species such as primate (e.g., human) and rodent (e.g., mouse) and looking for regions with high degrees of identity. Percent identity can be determined routinely using basic local alignment search tools (BLAST programs) (Altschul et al., J. Mol. Biol., 1990, 215, 403-410; Zhang and Madden, Genome Res., 1997, 7, 649-656), e.g., using the default parameters.

Once one or more target regions, segments or sites have been identified, e.g., within a sequence known in the art or provided herein, inhibitory nucleic acid compounds are chosen that are sufficiently complementary to the target, i.e., that hybridize sufficiently well and with sufficient specificity (i.e., do not substantially bind to other non-target RNAs), to give the desired effect.

In the context of this invention, hybridization means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as used herein, refers to the capacity for precise pairing between two nucleotides. For example, if a nucleotide at a certain position of an oligonucleotide is capable of hydrogen bonding with a nucleotide at the same position of a RNA molecule, then the inhibitory nucleic acid and the RNA are considered to be complementary to each other at that position. The inhibitory nucleic acids and the RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or precise pairing such that stable and specific binding occurs between the inhibitory nucleic acid and the RNA target. For example, if a base at one position of an inhibitory nucleic acid is capable of hydrogen bonding with a base at the corresponding position of a RNA, then the bases are considered to be complementary to each other at that position. 100% complementarity is not required.

It is understood in the art that a complementary nucleic acid sequence need not be 100% complementary to that of its target nucleic acid to be specifically hybridisable. A complementary nucleic acid sequence for purposes of the present methods is specifically hybridisable when binding of the sequence to the target RNA molecule interferes with the normal function of the target RNA to cause a loss of activity, and there is a sufficient degree of complementarity to avoid non-specific binding of the sequence to non-target RNA sequences under conditions in which specific binding is desired, e.g., under physiological conditions in the case of in vivo assays or therapeutic treatment, and in the case of in vitro assays, under conditions in which the assays are performed under suitable conditions of stringency. For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

In general, the inhibitory nucleic acids useful in the methods described herein have at least 80% sequence complementarity to a target region within the target nucleic acid, e.g., 90%, 95%, or 100% sequence complementarity to the target region within an RNA. For example, an antisense compound in which 18 of 20 nucleobases of the antisense oligonucleotide are complementary, and would therefore specifically hybridize, to a target region would represent 90 percent complementarity. Percent complementarity of an inhibitory nucleic acid with a region of a target nucleic acid can be determined routinely using basic local alignment search tools (BLAST programs) (Altschul et al., J. Mol. Biol., 1990, 215, 403-410; Zhang and Madden, Genome Res., 1997, 7, 649-656). Inhibitory nucleic acids that hybridize to an RNA can be identified through routine experimentation. In general the inhibitory nucleic acids must retain specificity for their target, i.e., must not directly bind to, or directly significantly affect expression levels of, transcripts other than the intended target.

For further disclosure regarding inhibitory nucleic acids, please see US2010/0317718 for antisense oligos; US2010/0249052 for double-stranded ribonucleic acid (dsRNA); US2009/0181914 and US2010/0234451 for LNAs; US2007/0191294 for siRNA analogues; US2008/0249039 for modified siRNA; and WO2010/129746 and WO2010/040112 for inhibitory nucleic acids, as well as WO2012/065143, WO 2012/087983, and WO 2014/025887 for inhibitory nucleic acids targeting non-coding RNAs/supRNAs; all of which are incorporated herein by reference in their entirety.

### Antisense

In some embodiments, the inhibitory nucleic acids are antisense oligonucleotides (ASOs). ASOs are typically designed to block expression of a DNA or RNA target by binding to the target and halting expression at the level of transcription, translation, or splicing. ASOs of the present invention are complementary nucleic acid sequences designed to hybridize under stringent conditions to an RNA. Thus, oligonucleotides are chosen that are sufficiently complementary to the target, i.e., that hybridize sufficiently well and with sufficient specificity, to confer the desired effect.

siRNA/shRNA

In some embodiments, the nucleic acid sequence that is complementary to an target RNA can be an interfering RNA, including but not limited to a small interfering RNA (“siRNA”) or a small hairpin RNA (“shRNA”). Methods for constructing interfering RNAs are well known in the art. For example, the interfering RNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure); the antisense strand comprises nucleotide sequence that is complementary to a nucleotide sequence in a target nucleic acid molecule or a portion thereof (i.e., an undesired gene) and the sense strand comprises nucleotide sequence corresponding to the target nucleic acid sequence or a portion thereof. Alternatively, interfering RNA is assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNA interference.

In some embodiments, the interfering RNA coding region encodes a self-complementary RNA molecule having a sense region, an antisense region and a loop region. Such an RNA molecule when expressed desirably forms a “hairpin” structure, and is referred to herein as an “shRNA.” The loop region is generally between about 2 and about 10 nucleotides in length. In some embodiments, the loop region is from about 6 to about 9 nucleotides in length. In some embodiments, the sense region and the antisense region are between about 15 and about 20 nucleotides in length. Following post-transcriptional processing, the small hairpin RNA is converted into a siRNA by a cleavage event mediated by the enzyme Dicer, which is a member of the RNase III family. The siRNA is then capable of inhibiting the expression of a gene with which it shares homology. For details, see Brummelkamp et al., Science 296:550-553, (2002); Lee et al, Nature Biotechnol., 20, 500-505, (2002); Miyagishi and Taira, Nature Biotechnol 20:497-500, (2002); Paddison et al. Genes & Dev. 16:948-958, (2002); Paul, Nature Biotechnol, 20, 505-508, (2002); Sui, Proc. Natl. Acad. Sd. USA, 99(6), 5515-5520, (2002); Yu et al. Proc Natl Acad Sci USA 99:6047-6052, (2002).

The target RNA cleavage reaction guided by siRNAs is highly sequence specific. In general, siRNA containing a nucleotide sequences identical to a portion of the target nucleic acid are preferred for inhibition. However, 100% sequence identity between the siRNA and the target gene is not required to practice the present invention. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Alternatively, siRNA sequences with nucleotide analog substitutions or insertions can be effective for inhibition. In general the siRNAs must retain specificity for their target, i.e., must not directly bind to, or directly significantly affect expression levels of, transcripts other than the intended target.

### Ribozymes

Trans-cleaving enzymatic nucleic acid molecules can also be used; they have shown promise as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the RNA non-functional.

In general, enzymatic nucleic acids with RNA cleaving activity act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

Several approaches such as in vitro selection (evolution) strategies (Orgel, 1979, Proc. R. Soc. London, B 205, 435) have been used to evolve new nucleic acid catalysts capable of catalyzing a variety of reactions, such as cleavage and ligation of phosphodiester linkages and amide linkages, (Joyce, 1989, Gene, 82, 83-87; Beaudry et al., 1992, Science 257, 635-641; Joyce, 1992, Scientific American 267, 90-97; Breaker et al, 1994, TIBTECH 12, 268; Bartel et al, 1993, Science 261:1411-1418; Szostak, 1993, TIBS 17, 89-93; Kumar et al, 1995, FASEB J., 9, 1183; Breaker, 1996, Curr. Op. Biotech., 1, 442). The development of ribozymes that are optimal for catalytic activity would contribute significantly to any strategy that employs RNA-cleaving ribozymes for the purpose of regulating gene expression. The hammerhead ribozyme, for example, functions with a catalytic rate (kcat) of about 1 min⁻¹ in the presence of saturating (10 rnM) concentrations of Mg²⁺ cofactor. An artificial “RNA ligase” ribozyme has been shown to catalyze the corresponding self-modification reaction with a rate of about 100 min⁻¹. In addition, it is known that certain modified hammerhead ribozymes that have substrate binding arms made of DNA catalyze RNA cleavage with multiple turn-over rates that approach 100 min⁻¹.

### Modified Inhibitory Nucleic Acids

In some embodiments, the inhibitory nucleic acids used in the methods described herein are modified, e.g., comprise one or more modified bonds or bases. A number of modified bases include phosphorothioate, methylphosphonate, peptide nucleic acids, or locked nucleic acid (LNA) molecules. Some inhibitory nucleic acids are fully modified, while others are chimeric and contain two or more chemically distinct regions, each made up of at least one nucleotide. These inhibitory nucleic acids typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric inhibitory nucleic acids of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures comprise, but are not limited to, U.S. Pat. Nos. 5,013,830; 5,149,797; 5, 220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the inhibitory nucleic acid comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make the inhibitory nucleic acid into which they are incorporated more resistant to nuclease digestion than the native oligodeoxynucleotide; these modified oligos survive intact for a longer time than unmodified inhibitory nucleic acids. Specific examples of modified inhibitory nucleic acids include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are inhibitory nucleic acids with phosphorothioate backbones and those with heteroatom backbones, particularly CH2-NH—O—CH2, CH,˜N(CH3)˜O˜CH2 (known as a methylene(methylimino) or MMI backbone], CH2-O—N(CH3)-CH2, CH2-N(CH3)-N(CH3)-CH2 and O—N(CH3)-CH2-CH2 backbones, wherein the native phosphodiester backbone is represented as O—P—O—CH,); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the inhibitory nucleic acid is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991.

Cyclohexenyl nucleic acid inhibitory nucleic acid mimetics are described in Wang et al., J. Am. Chem. Soc., 2000, 122, 8595-8602.

Modified inhibitory nucleic acid backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264, 562; 5, 264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

One or more substituted sugar moieties can also be included, e.g., one of the following at the 2′ position: OH, SH, SCH₃, F, OCN, OCH₃OCH₃, OCH₃O(CH₂)n CH₃, O(CH₂)n NH₂ or O(CH₂)n CH₃ where n is from 1 to about 10; Ci to C10 lower alkyl, alkoxyalkoxy, substituted lower alkyl, alkaryl or aralkyl; Cl; Br; CN; CF3; OCF3; O—, S—, or N-alkyl; O—, S—, or N-alkenyl; SOCH3; SO2 CH3; ONO2; NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an inhibitory nucleic acid; or a group for improving the pharmacodynamic properties of an inhibitory nucleic acid and other substituents having similar properties. A preferred modification includes 2′-methoxyethoxy [2′-0-CH₂CH₂OCH₃, also known as 2′-O-(2-methoxyethyl)] (Martin et al, HeIv. Chim. Acta, 1995, 78, 486). Other preferred modifications include 2′-methoxy (2′-O—CH₃), 2′-propoxy (2′-OCH₂ CH₂CH₃) and 2′-fluoro (2′-F). Similar modifications may also be made at other positions on the inhibitory nucleic acid, particularly the 3′ position of the sugar on the 3′ terminal nucleotide and the 5′ position of 5′ terminal nucleotide. Inhibitory nucleic acids may also have sugar mimetics such as cyclobutyls in place of the pentofuranosyl group.

Inhibitory nucleic acids can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine, can also be included. 5-Me-C substitutions have been shown to increase nucleic acid duplex stability by 0.6-1.2<0>C. (Sanghvi, Y. S., in Crooke, S. T. and Lebleu, B., eds., Antisense Research and Applications, CRC Press, Boca Raton, 1993, pp. 276-278) and are presently preferred base substitutions.

It is not necessary for all positions in a given inhibitory nucleic acid to be uniformly modified, and in fact more than one of the aforementioned modifications may be incorporated in a single inhibitory nucleic acid or even at within a single nucleoside within an inhibitory nucleic acid.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an inhibitory nucleic acid mimetic that has been shown to have excellent hybridization properties, is referred to as a peptide nucleic acid (PNA). In PNA compounds, the sugar-backbone of an inhibitory nucleic acid is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Inhibitory nucleic acids can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in ‘The Concise Encyclopedia of Polymer Science And Engineering’, pages 858-859, Kroschwitz, J. I., ed. John Wiley & Sons, 1990, those disclosed by Englisch et al., Angewandle Chemie, International Edition’, 1991, 30, page 613, and those disclosed by Sanghvi, Y. S., Chapter 15, Antisense Research and Applications’, pages 289-302, Crooke, S. T. and Lebleu, B. ea., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2<0>C (Sanghvi, Y. S., Crooke, S. T. and Lebleu, B., eds, ‘Antisense Research and Applications’, CRC Press, Boca Raton, 1993, pp. 276-278) and are presently preferred base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175, 273; 5, 367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

In some embodiments, the inhibitory nucleic acids are chemically linked to one or more moieties or conjugates that enhance the activity, cellular distribution, or cellular uptake of the inhibitory nucleic acid. Such moieties comprise but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S— tritylthiol (Manoharan et al, Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther., 1996, 277, 923-937). See also U.S. Pat. Nos. 4,828,979; 4,948,882; 5,218,105; 5,525,465; 5,541,313; 5,545,730; 5,552, 538; 5,578,717, 5,580,731; 5,580,731; 5,591,584; 5,109,124; 5,118,802; 5,138,045; 5,414,077; 5,486, 603; 5,512,439; 5,578,718; 5,608,046; 4,587,044; 4,605,735; 4,667,025; 4,762, 779; 4,789,737; 4,824,941; 4,835,263; 4,876,335; 4,904,582; 4,958,013; 5,082, 830; 5,112,963; 5,214,136; 5,082,830; 5,112,963; 5,214,136; 5, 245,022; 5,254,469; 5,258,506; 5,262,536; 5,272,250; 5,292,873; 5,317,098; 5,371,241, 5,391, 723; 5,416,203, 5,451,463; 5,510,475; 5,512,667; 5,514,785; 5, 565,552; 5,567,810; 5,574,142; 5,585,481; 5,587,371; 5,595,726; 5,597,696; 5,599,923; 5,599, 928 and 5,688,941, each of which is herein incorporated by reference.

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

### Locked Nucleic Acids (LNAs)

In some embodiments, the modified inhibitory nucleic acids used in the methods described herein comprise locked nucleic acid (LNA) molecules, e.g., including [alpha]-L-LNAs. LNAs comprise ribonucleic acid analogues wherein the ribose ring is “locked” by a methylene bridge between the 2′-oxgygen and the 4′-carbon—i.e., inhibitory nucleic acids containing at least one LNA monomer, that is, one 2′-O,4′-C-methylene-β-D-ribofuranosyl nucleotide. LNA bases form standard Watson-Crick base pairs but the locked configuration increases the rate and stability of the basepairing reaction (Jepsen et al., Oligonucleotides, 14, 130-146 (2004)). LNAs also have increased affinity to base pair with RNA as compared to DNA. These properties render LNAs especially useful as probes for fluorescence in situ hybridization (FISH) and comparative genomic hybridization, as knockdown tools for miRNAs, and as antisense oligonucleotides to target mRNAs or other RNAs, e.g., RNAs as described herein.

The LNA molecules can include molecules comprising 10-30, e.g., 12-24, e.g., 12, 13, 14, 15, 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the RNA. The LNA molecules can be chemically synthesized using methods known in the art.

The LNA molecules can be designed using any method known in the art; a number of algorithms are known, and are commercially available (e.g., on the internet, for example at exiqon.com). See, e.g., You et al., Nuc. Acids. Res. 34:e60 (2006); McTigue et al., Biochemistry 43:5388-405 (2004); and Levin et al., Nuc. Acids. Res. 34:e142 (2006). For example, “gene walk” methods, similar to those used to design antisense oligos, can be used to optimize the inhibitory activity of the LNA; for example, a series of inhibitory nucleic acids of 10-30 nucleotides spanning the length of a target RNA can be prepared, followed by testing for activity. Optionally, gaps, e.g., of 5-10 nucleotides or more, can be left between the LNAs to reduce the number of inhibitory nucleic acids synthesized and tested. GC content is preferably between about 30-60%. General guidelines for designing LNAs are known in the art; for example, LNA sequences will bind very tightly to other LNA sequences, so it is preferable to avoid significant complementarity within an LNA. Contiguous runs of more than four LNA residues, should be avoided where possible (for example, it may not be possible with very short (e.g., about 9-10 nt) inhibitory nucleic acids). In some embodiments, the LNAs are xylo-LNAs.

For additional information regarding LNAs see U.S. Pat. Nos. 6,268,490; 6,734,291; 6,770,748; 6,794,499; 7,034,133; 7,053,207; 7,060,809; 7,084,125; and 7,572,582; and U.S. Pre-Grant Pub. Nos. 20100267018; 20100261175; and 20100035968; Koshkin et al. Tetrahedron 54, 3607-3630 (1998); Obika et al. Tetrahedron Lett. 39, 5401-5404 (1998); Jepsen et al., Oligonucleotides 14:130-146 (2004); Kauppinen et al., Drug Disc. Today 2(3):287-290 (2005); and Ponting et al., Cell 136(4):629-641 (2009), and references cited therein.

### Making and Using Inhibitory Nucleic Acids

The nucleic acid sequences used to practice the methods described herein, whether RNA, cDNA, genomic DNA, vectors, viruses or hybrids thereof, can be isolated from a variety of sources, genetically engineered, amplified, and/or expressed/generated recombinantly. Recombinant nucleic acid sequences can be individually isolated or cloned and tested for a desired activity. Any recombinant expression system can be used, including e.g. in vitro, bacterial, fungal, mammalian, yeast, insect or plant cell expression systems.

Nucleic acid sequences of the invention can be inserted into delivery vectors and expressed from transcription units within the vectors. The recombinant vectors can be DNA plasmids or viral vectors. Generation of the vector construct can be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example as described in Sambrook et al. Molecular Cloning: A Laboratory Manual. (1989)), Coffin et al. (Retroviruses. (1997)) and “RNA Viruses: A Practical Approach” (Alan J. Cann, Ed., Oxford University Press, (2000)). As will be apparent to one of ordinary skill in the art, a variety of suitable vectors are available for transferring nucleic acids of the invention into cells. The selection of an appropriate vector to deliver nucleic acids and optimization of the conditions for insertion of the selected expression vector into the cell, are within the scope of one of ordinary skill in the art without the need for undue experimentation. Viral vectors comprise a nucleotide sequence having sequences for the production of recombinant virus in a packaging cell. Viral vectors expressing nucleic acids of the invention can be constructed based on viral backbones including, but not limited to, a retrovirus, lentivirus, adenovirus, adeno-associated virus, pox virus or alphavirus. The recombinant vectors capable of expressing the nucleic acids of the invention can be delivered as described herein, and persist in target cells (e.g., stable transformants).

Nucleic acid sequences used to practice this invention can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Nucleic acid sequences of the invention can be stabilized against nucleolytic degradation such as by the incorporation of a modification, e.g., a nucleotide modification. For example, nucleic acid sequences of the invention includes a phosphorothioate at least the first, second, or third internucleotide linkage at the 5′ or 3′ end of the nucleotide sequence. As another example, the nucleic acid sequence can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., comprise nucleic acid analogues in which the ribose ring is “locked” by a methylene bridge connecting the 2′-O atom and the 4′-C atom (see, e.g., Kaupinnen et al., Drug Disc. Today 2(3):287-290 (2005); Koshkin et al., J. Am. Chem. Soc., 120(50):13252-13253 (1998)). For additional modifications see US 20100004320, US 20090298916, and US 20090143326.

Techniques for the manipulation of nucleic acids used to practice this invention, such as, e.g., subcloning, labeling probes (e.g., random-primer labeling using Klenow polymerase, nick translation, amplification), sequencing, hybridization and the like are well described in the scientific and patent literature, see, e.g., Sambrook et al., Molecular Cloning; A Laboratory Manual 3d ed. (2001); Current Protocols in Molecular Biology, Ausubel et al., eds. (John Wiley & Sons, Inc., New York 2010); Kriegler, Gene Transfer and Expression: A Laboratory Manual (1990); Laboratory Techniques In Biochemistry And Molecular Biology: Hybridization With Nucleic Acid Probes, Part I. Theory and Nucleic Acid Preparation, Tijssen, ed. Elsevier, N.Y. (1993).

### Pharmaceutical Compositions

The methods described herein can include the administration of pharmaceutical compositions and formulations comprising an inhibitor of XIST RNA and an inhibitor of an Xist-interacting protein, e.g., a chromatin-modifying protein, e.g., a small molecule inhibitor or an inhibitory nucleic acid such as a small inhibitory RNA (siRNA) or LNA that targets XIST RNA and/or a gene encoding Xist or an Xist-interacting protein, e.g., a chromatin-modifying protein, and optionally an inhibitory nucleic acid that specifically binds, or is complementary, to a strong or moderate binding site or a supRNA described in WO 2012/065143, WO 2012/087983, WO 2014/025887 and U.S. Ser. No. 62/010,342. The methods can include administration of a single composition comprising an inhibitor of Xist and an inhibitor of an Xist-interacting protein, e.g., a chromatin-modifying protein, or multiple compositions, e.g., each comprising one or both of an inhibitor of Xist and an inhibitor of an Xist-interacting protein, e.g., a chromatin-modifying protein.

In some embodiments, the compositions are formulated with a pharmaceutically acceptable carrier. The pharmaceutical compositions and formulations can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005.

The inhibitory nucleic acids can be administered alone or as a component of a pharmaceutical formulation (composition). The compounds may be formulated for administration, in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compositions of the invention include those suitable for intradermal, inhalation, oral/nasal, topical, parenteral, rectal, and/or intravaginal administration. The formulations may conveniently be presented in unit dosage form and may be prepared by any methods well known in the art of pharmacy. The amount of active ingredient (e.g., nucleic acid sequences of this invention) which can be combined with a carrier material to produce a single dosage form will vary depending upon the host being treated, the particular mode of administration, e.g., intradermal or inhalation. The amount of active ingredient which can be combined with a carrier material to produce a single dosage form will generally be that amount of the compound which produces a therapeutic effect, e.g., an antigen specific T cell or humoral response.

Pharmaceutical formulations can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., nucleic acid sequences of the invention) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

In some embodiments, oil-based pharmaceuticals are used for administration of nucleic acid sequences of the invention. Oil-based suspensions can be formulated by suspending an active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102.

Pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, these injectable oil-in-water emulsions of the invention comprise a paraffin oil, a sorbitan monooleate, an ethoxylated sorbitan monooleate and/or an ethoxylated sorbitan trioleate.

The pharmaceutical compounds can also be administered by in intranasal, intraocular and intravaginal routes including suppositories, insufflation, powders and aerosol formulations (for examples of steroid inhalants, see e.g., Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In some embodiments, the pharmaceutical compounds can be delivered transdermally, by a topical route, formulated as applicator sticks, solutions, suspensions, emulsions, gels, creams, ointments, pastes, jellies, paints, powders, and aerosols.

In some embodiments, the pharmaceutical compounds can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In some embodiments, the pharmaceutical compounds can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

In some embodiments, the pharmaceutical compounds and formulations can be lyophilized. Stable lyophilized formulations comprising an inhibitory nucleic acid can be made by lyophilizing a solution comprising a pharmaceutical of the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. 20040028670.

The compositions and formulations can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587. As used in the present invention, the term “liposome” means a vesicle composed of amphiphilic lipids arranged in a bilayer or bilayers. Liposomes are unilamellar or multilamellar vesicles that have a membrane formed from a lipophilic material and an aqueous interior that contains the composition to be delivered. Cationic liposomes are positively charged liposomes that are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes can also include “sterically stabilized” liposomes, i.e., liposomes comprising one or more specialized lipids. When incorporated into liposomes, these specialized lipids result in liposomes with enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

The formulations of the invention can be administered for prophylactic and/or therapeutic treatments. In some embodiments, for therapeutic applications, compositions are administered to a subject who is need of reduced triglyceride levels, or who is at risk of or has a disorder described herein, in an amount sufficient to cure, alleviate or partially arrest the clinical manifestations of the disorder or its complications; this can be called a therapeutically effective amount. For example, in some embodiments, pharmaceutical compositions of the invention are administered in an amount sufficient to decrease serum levels of triglycerides in the subject.

The amount of pharmaceutical composition adequate to accomplish this is a therapeutically effective dose. The dosage schedule and amounts effective for this use, i.e., the dosing regimen, will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; Remington: The Science and Practice of Pharmacy, 21st ed., 2005). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on for example: the dosage and frequency as required and tolerated by the patient, the degree and amount of therapeutic effect generated after each administration (e.g., effect on tumor size or growth), and the like. The formulations should provide a sufficient quantity of active agent to effectively treat, prevent or ameliorate conditions, diseases or symptoms.

In alternative embodiments, pharmaceutical formulations for oral administration are in a daily amount of between about 1 to 100 or more mg per kilogram of body weight per day. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington: The Science and Practice of Pharmacy, 21st ed., 2005.

Various studies have reported successful mammalian dosing using complementary nucleic acid sequences. For example, Esau C., et al., (2006) Cell Metabolism, 3(2):87-98 reported dosing of normal mice with intraperitoneal doses of miR-122 antisense oligonucleotide ranging from 12.5 to 75 mg/kg twice weekly for 4 weeks. The mice appeared healthy and normal at the end of treatment, with no loss of body weight or reduced food intake. Plasma transaminase levels were in the normal range (AST ¾ 45, ALT ¾ 35) for all doses with the exception of the 75 mg/kg dose of miR-122 ASO, which showed a very mild increase in ALT and AST levels. They concluded that 50 mg/kg was an effective, non-toxic dose. Another study by Krutzfeldt J., et al., (2005) Nature 438, 685-689, injected anatgomirs to silence miR-122 in mice using a total dose of 80, 160 or 240 mg per kg body weight. The highest dose resulted in a complete loss of miR-122 signal. In yet another study, locked nucleic acids (“LNAs”) were successfully applied in primates to silence miR-122. Elmen J., et al., (2008) Nature 452, 896-899, report that efficient silencing of miR-122 was achieved in primates by three doses of 10 mg kg-1 LNA-antimiR, leading to a long-lasting and reversible decrease in total plasma cholesterol without any evidence for LNA-associated toxicities or histopathological changes in the study animals.

In some embodiments, the methods described herein can include co-administration with other drugs or pharmaceuticals, e.g., compositions for providing cholesterol homeostasis. For example, the inhibitory nucleic acids can be co-administered with drugs for treating or reducing risk of a disorder described herein.

Disorders Associated with X-Inactivation

The present disclosure provides methods for treating X-linked diseases formulated by administering an inhibitor of an XIST RNA and an inhibitor of an Xist interacting protein, e.g., a small molecule inhibitor or an inhibitory nucleic acid such as a small inhibitory RNA (siRNA) or LNA that targets XIST or a gene encoding XIST or an Xist-interacting protein, e.g., a chromatin-modifying protein, and optionally an inhibitory nucleic acid that specifically binds, or is complementary, to a strong or moderate binding site or a supRNA described in WO 2012/065143, WO 2012/087983, WO 2014/025887 and U.S. Ser. No. 62/010,342, to disrupt silencing of genes controlled by the PRC2 sites (e.g., all of the genes within a cluster), or to disrupt silencing of one specific gene. This methodology is useful in X-linked disorders, e.g., in heterozygous women who retain a wild-type copy of a gene on the Xi (See, e.g., Lyon, Acta Paediatr Suppl. 2002; 91(439):107-12; Carrell and Willard, Nature. 434(7031):400-4 (2005); den Veyver, Semin Reprod Med. 19(2):183-91 (2001)). In females, reactivating a non-disease silent allele on the Xi would be therapeutic in many cases of X-linked disease, such as Rett Syndrome (caused by MECP2 mutations), Fabry's Disease (caused by GLA mutations), or X-linked hypophosphatemia (caused by mutation of PHEX). The methodology may also be utilized to treat male X-linked disease. In both females and males, upregulation of a hypomorphic or epigenetically silenced allele may alleviate disease phenotype, such as in Fragile X Syndrome, where the mechanism of epigenetic silencing of FMR1 may be similar to epigenetic silencing of a whole Xi in having many different types of heterochromatic marks.

As a result of X-inactivation, heterozygous females are mosaic for X-linked gene expression; some cells express genes from the maternal X and other cells express genes from the paternal X. The relative ratio of these two cell populations in a given female is frequently referred to as the “X-inactivation pattern.” One cell population may be at a selective growth disadvantage, resulting in clonal outgrowth of cells with one or the other parental X chromosome active; this can cause significant deviation or skewing from an expected mean X-inactivation pattern (i.e., 50:50). See, e.g., Plenge et al., Am. J. Hum. Genet. 71:168-173 (2002) and references cited therein.

The present methods can be used to treat disorders associated with X-inactivation, which includes those listed in Table 2. The methods include administering a an inhibitor of XIST RNA (e.g., an inhibitory nucleic acid such as a small inhibitory RNA (siRNA) or LNA that targets Xist) and an inhibitor of an Xist-interacting protein, e.g., a chromatin-modifying protein, e.g., a small molecule inhibitor, and optionally an inhibitory nucleic acid that specifically binds, or is complementary, to a strong or moderate binding site or a supRNA described in WO 2012/065143, WO 2012/087983, WO 2014/025887 and U.S. Ser. No. 62/010,342, i.e., a supRNA associated with the gene that causes the disorder, as shown in Table 2 and WO 2012/065143, WO 2012/087983, and WO 2014/025887.

TABLE 2
X Linked Disorders and Associated Genes

| Disorder | OMIM # | Locus | Gene |
|---|---|---|---|
| Dent's disease 1 | 300009 | Xp11.22 | CLCN5 |
| Testicular feminization syndrome | 300068 | Xq11-q12 | AR |
| Addison's disease with cerebral | 300100 | Xq28 | ABCD1 |
| sclerosis | | | |
| Adrenal hypoplasia | 300200 | XP21.3-p21.2 | DAX1 |
| siderius X-linked mental retardation | 300263 | Xp11.22 | PHF8 |
| syndrome | | | |
| Agammaglobulinaemia, Bruton type | 300300 | Xq21.3-q22 | BTK |
| Choroidoretinal degeneration | 300389 | Xp21.1 | RPGR |
| Choroidaemia | 300390 | Xq21.2 | CHM |
| Albinism, ocular | 300500 | Xp22.3 | OA1 |
| Dent's disease 2 | 300555 | Xq25-q26 | OCRL |
| fragile X syndrome | 300624 | Xq27.3 | FMR1 |
| Rett/Epileptic encephalopathy, early | 300672 | Xp22.13 | CDKL5 |
| infantile, 2 | | | |
| Albinism-deafness syndrome | 300700 | Xq26.3-q27.1 | ADFN |
| paroxysmal nocturnal hemoglobinuria | 300818 | Xp22.2 | PIGA |
| Aldrich syndrome | 301000 | Xp11.23-p11.22 | WAS |
| Alport syndrome | 301050 | Xq22.3 | COL4A5 |
| Anaemia, hereditary hypochromic | 301300 | Xp11.21 | ALAS2 |
| Anemia, sideroblastic, with ataxia | 301310 | Xq13.3 | ABCB7 |
| Fabry disease | 301500 | Xq22 | GLA |
| Spinal muscular atrophy 2 | 301830 | Xp11.23 | UBA1 |
| Cataract, congenital | 302200 | Xp | CCT |
| Charcot-Marie-Tooth, peroneal | 302800 | Xq13.1 | GJB1 |
| Spastic paraplegia | 303350 | Xq28 | L1CAM |
| Colour blindness | 303800 | Xq28 | OPN1MW |
| Diabetes insipidus, nephrogenic | 304800 | Xq28 | AVPR2 |
| Dyskeratosis congenita | 305000 | Xq28 | DKC1 |
| Ectodermal dysplasia, anhidrotic | 305100 | Xq12-q13.1 | ED1 |
| Faciogenital dysplasia (Aarskog | 305400 | Xp11.21 | FGD1 |
| syndrome) | | | |
| Glucose-6-phosphate dehydrogenase | 305900 | Xq28 | G6PD |
| deficiency | | | |
| Glycogen storage disease type VIII | 306000 | Xp22.2-p22.1 | PHKA2 |
| Gonadal dysgenesis (XY female type) | 306100 | Xp22.11-p21.2 | GDXY |
| Granulomatous disease (chronic) | 306400 | Xp21.1 | CYBB |
| Haemophilia A | 306700 | Xq28 | F8 |
| Haemophilia B | 306900 | Xq27.1-q27.2 | F9 |
| Hydrocephalus (aqueduct stenosis) | 307000 | Xq28 | L1CAM |
| Hypophosphataemic rickets | 307800 | Xp22.2-p22.1 | PHEX |
| Lesch-Nyhan syndrome (hypoxanthine- | 308000 | Xq26-q27.2 | HPRT1 |
| guanine-phosphoribosyl transferase | | | |
| deficiency) | | | |
| Incontinentia pigmenti | 308300 | Xq28 | IKBKG |
| Kallmann syndrome | 308700 | Xp22.3 | KAL1 |
| Keratosis follicularis spinulosa | 308800 | Xp22.1 | SAT |
| Lowe (oculocerebrorenal) syndrome | 309000 | Xq26.1 | OCRL |
| Menkes syndrome | 309400 | Xq12-q13 | ATP7A |
| Renpenning Syndrome | 309500 | Xp11.23 | PQBP1 |
| Mental retardation, with or without | 309530 | Xp11.3-q21.1 | MRX1 |
| fragile site (numerous specific types) | | | |
| Coffin-Lowry syndrome | 309580 | Xq13 | ATRX |
| Microphthalmia with multiple | 309800 | Xq27-q28 | MAA |
| anomalies (Lenz syndrome) | | | |
| Muscular dystrophy (Becker, Duchenne | 310300 | Xq28 | EMD |
| and Emery-Dreifuss types) | | | |
| Myotubular myopathy | 310400 | Xq28 | MTM1 |
| Night blindness, congenital stationary | 310500 | Xp11.4 | CSNB1 |
| Norrie's disease (pseudoglioma) | 310600 | Xp11.4 | NDP |
| Nystagmus, oculomotor or ‘jerky’ | 310700 | Xq26-q27 | NYS1 |
| Orofaciodigital syndrome (type I) | 311200 | Xp22.3-p22.2 | OFD1 |
| Ornithine transcarbamylase deficiency | 311250 | Xp21.1 | OTC |
| (type I hyperammonaemia) | | | |
| Phosphoglycerate kinase deficiency | 311800 | Xq13 | PGK1 |
| Phosphoribosylpyrophosphate | 311850 | Xq22-q24 | PRPS1 |
| synthetase deficiency | | | |
| Retinitis pigmentosa | 312610 | Xp21.1 | RPGR |
| Retinoschisis | 312700 | Xp22.2-p22.1 | RS1 |
| Rett syndrome | 312750 | Xq28, Xp22 | MECP2 |
| Muscular atrophy/Dihydrotestosterone | 313200 | Xq11-q12 | AR |
| receptor deficiency | | | |
| Spinal muscular atrophy | 313200 | Xq11-q12 | AR |
| Spondyloepiphyseal dysplasia tarda | 313400 | Xp22.2-p22.1 | SEDL |
| Thrombocytopenia, hereditary | 313900 | Xp11.23-p11.22 | WAS |
| Thyroxine-binding globulin, absence | 314200 | Xq22.2 | TBG |
| McLeod syndrome | 314850 | Xp21.1 | XK |

Table 2 was adapted in part from Germain, “Chapter 7: General aspects of X-linked diseases” in Fabry Disease: Perspectives from 5 Years of FOS. Mehta A, Beck M, Sunder-Plassmann G, editors. (Oxford: Oxford PharmaGenesis; 2006).

### EXAMPLES

The invention is further described in the following examples, which do not limit the scope of the invention described in the claims.

Materials and Methods

The following materials and methods were used in the Examples, below. No statistical methods were used to predetermine sample size, the experiments were not randomized and the investigators were not blinded to allocation during experiments and outcome assessment.

### Design of Gapmers

Gapmers targeting Xist were designed following specific design algorithms (Exiqon), sequences in Table S-I. 5-aza-2′-deoxycytidine (Aza) and other small molecules were purchased from Selleckchem or Tocris. I-BRD9 was obtained from SGC.

TABLE S-I
ASO sequences

| Name | Sequence (5′ to 3′) | SEQ ID NO: |
|---|---|---|
| Scramble ASO | AACACGTCTATACGC | 1. |
| Xist ASO 1, a | TCTTGGTTACTAACAG | 2. |
| Xist ASO 2 | AGTAGCTCGGTGGAT | 3. |
| Xist ASO 3 | TGAGTCTTGAGGAGAA | 4. |
| b | CGGTGCCTAGCATGCA | 5. |
| c | GTTGATTGATAGGA | 6. |
| d | GAAAGGCACGAAGAG | 7. |
| e | GGATGCTTGAAACA | 8. |
| f | TGGAAAGGAGGAGGTG | 9. |
| g | GTGTATGCGTGTGA | 10. |
| h | CCTTACCCCTCTAAAA | 11. |
| i | AGGAGCTTAAAGTGAG | 12. |
| j | GGTTGAAAGGAGGAAC | 13. |
| k | TTCAGCACTACACAT | 14. |
| l | AGATCAGGATGTAGC | 15. |
| m | GCAGGAAGGAAGCTG | 16. |
| n | CGATAGATATGAGAC | 17. |
| o | GATGTTTCTGCTTTG | 18. |
| p | CATCCACTCATCCTT | 19. |
| q | TCAGTCCCTCACTCC | 20. |
| r | TCTGTGGTTGTTTTC | 21. |
| s | CTTTCTCTCAATTCC | 22. |

### Tissue Culture

Mecp2-Luc fibroblast cell lines were a generous gift from Dr. Bedalov. The clonal hybrid (cast/mus) cell line (EY.T4) was previously developed in the lab (Yildirim E et al. (2011) X-chromosome hyperactivation in mammals via nonlinear relationships between chromatin states and transcription. Nat Struct Mol Biol 19:56-61). Passage number was kept below 25, no further verification of cell line identity was performed. They were maintained in DMEM-glutamax (Gibco), supplemented with fetal bovine serum (FBS, 10%), non-essential amino acids (1×, Gibco), HEPES buffer (25 mM, Gibco), penicillin/streptomycin (1×, Gibco) and 2-Mercaptoethanol (Sigma). Transfection was performed with 20 nM ASO assisted by Lipofectamine LTX with Plus reagent (Thermo Fisher).

Imaging was done with a Nikon Eclipse TE2000-E equipped with a HamamatsuCCD camera. Image analysis was done with OpenLAB software (Agilent).

### Luciferase Assay

Immortalized clonal MEF cell line that carries an Mecp2:luciferase fusion gene on the Xi were used for conducting these experiments (Sripathy S, et al. (2017) Screen for reactivation of MeCP2 on the inactive X chromosome identifies the BMP/TGF-beta superfamily as a regulator of XIST expression. Proc Natl Acad Sci USA 114:1619-1624). Xa-Mecp2-Luc clone cell line was used in parallel for providing a scaling magnitude for normalizing Xi-driven luciferase signals, Cells were grown in a 12 well plate, trypsinized, counted, washed with PBS and dispensed in 20 ul of 1× cell culture lysis reagent (Promega). The mixture was vortexed and incubated for 5 min and then transferred to a zebra 96 well plate. The plate was read using a Perkin Elmer MicroBeta2 LumiJET that automatically adds 100 μl of Luciferase Assay Reagent (Promega) 2 sec before measuring the produced light for 10 sec. The corrected counts per second where divided by the number of cells for generating a luciferase-reactivation score per cell. For the initiating screen ASOs were used at 20 nM (transfected with lipofectamine) in combination with 0.5 uM Aza for 3 days. The reverse screen used 20 nM Xist ASO (transfected with lipofectamine) in combination with the small molecule inhibitors at different concentrations (see Table S-II) for 3 days.

TABLE S-II
concentrations and targets of the small molecules in the screen

| Inhibitor | Concentra- | |
|---|---|---|
| Name | tion (uM) | Target |
| etoposide | 0.25 | DNA topoisomerase 2 (TOP2) |
| EPZ6438 | 1 | Enhancer of zeste homolog 2 (EZH2) |
| PFI-3 | 5 | Transcription activator BRG1 (SMARCA4) |
| PRT4165 | 10 | Ring Finger Protein 1 (RING1) |
| I-BRD9 | 0.5 | Bromodomain Containing 9 (BRD9) |
| A366 | 0.05 | Euchromatic histone-lysine N- |
| | | methyltransferase 2 (G9A) |
| SGC-CBP30 | 0.5 | CREB Binding Protein (CREBBP) |
| olaparib | 0.05 | Poly ADP ribose polymerase (PARP) |
| JQ1 | 0.5 | Bromodomain and extraterminal domain |
| | | (BET) |
| barasertib | 0.01 | Aurora Kinase B (AURKB) |
| topotecan | 0.01 | DNA topoisomerase 1 (TOP1) |
| entinostat | 0.5 | Histone deacetylase 1&3 (HDAC1 & 3) |
| tubastatin a | 0.5 | Histone deacetylase 6 (HDAC6) |
| bestatin | 0.5 | Aminopeptidase |
| romidepsin | 0.5 | Histone deacetylase 1&2 (HDAC1 & 2) |
| rocilinostat | 0.05 | Histone deacetylase 6 (HDAC6) |
| vorinostat | 0.5 | Histone deacetylase (HDAC) |
| Aza | 0.5 | DNA methyltransferase 1 (DNMT1) |
| valproic acid | 0.5 | Histone deacetylase (HDAC) |
| VX680 | 1 | Aurora Kinase (AURK) |

qPCR

RNA was isolated by Trizol (Life Technologies) extraction (tissues were snap frozen in liquid nitrogen and ground with pestle and mortar or put in Trizol and homogenized using a Qiagen TissueLyser), treated with TurboDNAse for 30 min at 37° C. 2 μg RNA was used for each of the reverse transcriptase (and rt minus control) reactions (Superscript III, Invitrogen) followed by the SYBR green qPCR using the primers listed in table, with annealing temperature of 60° C. for 45 cycles. The relative efficiency of the reactivations was calculated by comparing to GAPDH or TBP RNA as the internal control.

### RNA-Seq

Strand-specific RNA-seq was performed as previously described (Kung J T et al. (2015) Locus-specific targeting to the X chromosome revealed by the RNA interactome of CTCF. Mol Cell 57:361-375; Minajigi A et al. (2015) A comprehensive Xist interactome reveals cohesin repulsion and an RNA-directed chromosome conformation. Science, aab2276-12). All libraries were sequenced with Illumina HiSeq, generating 28-54 millions paired-end 50 nucleotide reads per sample. RNA-seq reads were aligned allele-specifically to 129S1/SvJm (mus) and CAST/Eih (cas) genome using TopHat2 (Kim D, et al. (2013) TopHat2: Accurate alignment of transcriptomes in the presence of insertions, deletions and gene fusions. Genome Biol 14:R36). After removal of PCR duplicates, all unique reads mapped to the exons of each gene were quantified by Homer (Heinz S, et al. (2010) Simple combinations of lineage-determining transcription factors prime cis-regulatory elements required for macrophage and B cell identities. Mol Cell 38:576-589). For non-allelic analysis, we used all reads (comp reads, which contain both allele-specific reads and reads that do not overlap with SNPs) to perform normalized differential expression analyses by DESeq (Anders S, Huber W (2010) Differential expression analysis for sequence count data. Genome Biol 11:R106). To compare the fold change of autosomal and X-linked genes, only genes with FPKM≥1 were considered (Yang L, Kirby J E, Sunwoo H, Lee J T (2016) Female mice lacking Xist RNA show partial dosage compensation and survive to term. Genes Dev 30:1747-1760). Upregulated genes were defined as genes with fold change>1.2. For allele-specific analysis, we defined % mus as the percentage of mus-specific exonic reads in all allele-specific (mus-specific+cas-specific) exonic reads of each transcript. For classification of X-linked genes, we defined expressed genes as genes having non-zero FPKM in all samples. Allele-assessable genes were defined as active genes that have more than 12 allele-specific reads in all samples (Pinter S F and Colognori D (2015) Allelic imbalance is a prevalent and tissue-specific feature of the mouse transcriptome. Genetics 200:537-549). It has been described that a small fraction of genes overlap with incorrectly annotated SNPs and produce unexpected allelic skewing (Pinter S F and Colognori D (2015) Allelic imbalance is a prevalent and tissue-specific feature of the mouse transcriptome. Genetics 200:537-549; Calabrese J M et al. (2012) Site-specific silencing of regulatory elements as a mechanism of X inactivation. Cell 151:951-963). These genes were identified by analyzing a published RNA-seq dataset of tail-tip fibroblasts (TTF) from pure Mus castaneous background. Allele-assessable genes having % mus greater than 9.09% in the pure cas TTF were considered as genes with miscalled SNPs. Genes that are qualified for allele-specific analysis (qualified genes) were defined as genes that were allele-assessable and were not genes with miscalled SNPs. Among qualified genes, we defined escapees as genes whose expression from the Xi is greater than 10% of the expression from the Xa (Yang F, Babak T, Shendure J, Disteche C M (2010) Global survey of escape from X inactivation by RNA-sequencing in mouse. Genome Res 20:614-622) in wild-type hybrid MEF treated with control ASO. Genes subjected to X-inactivation (X-inactivated genes) were defined as expressed and allele-assessable genes that were not genes with miscalled SNPs and escapees. The cumulative distribution plots, histograms, heat maps, and scatter plots were constructed with R, ggplot2, and Gviz package (http://www.R-project.org). To visualize RNA-seq coverage, we generated strand-resolved fpm-normalized bigWig files from the raw RNA-seq reads for all reads (comp), mus-specific (mus) reads, and cas-specific (cas) reads separately, which were displayed using IGV with scales indicated in each tract.

### Mice Husbandry

Mouse husbandry was carried out as stipulated by the Massachusetts Hospital Institutional Animal Care and Use Committee (IACUC) and all animal experiments were approved by them. Moribund animals (sacrificed per IACUC) were included in the data as deceased animals.

Xist2lox/Xist2lox mice (129Sv/Jae strain) were a gift of R. Jaenisch (64). Nestin-Cre mice (B6.Cg-Tg(Nes-cre)1Kln/J) were a gift from R. Kelleher. To generate XistΔ/+ mice, we crossed Xist2lox/Xist2lox females to Nest-Cre males. To generate homozygous mutants, we crossed Xist2lox/Xist2lox females to Xist2lox/Y; Nest-Cre males. Mice were screened by PCR for Nest-Cre and Xist2lox alleles using the primers in Table S-III.

TABLE S-III
primers for qPCR

| Name | Sequence (5′ to 3′) | SEQ ID NO: |
|---|---|---|
| Tbp F | ACGGACAACTGCGTTGATTTT | 23. |
| Tbp R | ACTTAGCTGGGAAGCCCAAC | 24. |
| GapdH F | ATGAATACGGCTACAGCAACAGG | 25. |
| GapdH R | CTCTTGCTCAGTGTCCTTGCTG | 26. |
| Xist F | CAGAGTAGCGAGGACTTGAAGAG | 27. |
| Xist R | GCTGGTTCGTCTATCTTGTGGG | 28. |
| Luc F | TCTAAGGAAGTCGGGGAAGC | 29. |
| Luc R | CCCTCGGGTGTAATCAGAAT | 30. |

TABLE S-IV
primers for mouse genotyping

| Name | Sequence (5′ to 3′) | SEQ ID NO: |
|---|---|---|
| Xist F | GTGCCATATCAGTGAGCTCTCG | 31. |
| Xist2lox R | AACCAAGGTTGAGAGAGCAAA | 32. |
| Xist1lox R | TGTCACCTACCAATGAGAGATCC | 33. |
| Cre F | GCGGTCTGGCAGTAAAAACTATC | 34. |
| Cre R | GTGAAACAGCATTGCTGTCACTT | 35. |
| Cre ICF | CTAGGCCACAGAATTGAAAGATCT | 36. |
| Cre ICR | GTAGGTGGAAATTCTAGCATCATCC | 37. |
| Mecp2 F | AAATTGGGTTACACCGCTGA | 38. |
| Mecp2 R mut | CCACCTAGCCTGCCTGTACT | 39. |
| Mecp2 R WT | CTGTATCCTTGGGTCAAGCTG | 40. |
| Tsix F | GGAGAAGCCATTTTCCATCA | 41. |
| Tsix mut R | ACGGAACGCAGTACCAAAAT | 42. |
| Tsix WT R | CAAAAATCCCCAAGAATGTGA | 43. |
| Neo F | CGTTGGCTACCCGTGATATT | 44. |
| Neo R | TCAGAAGAACTCGTCAAGAAGG | 45. |
| Sex X F | GGTAACAATTTTCCCGCCATGTG | 46. |
| Sex X R | GGAAATAAACGGAACGCAGTACC | 47. |
| Sex Y F | GACTAGACATGTCTTAACATCTGTCC | 48. |
| Sex Y R | CCTATTGCATGGACAGCAGCTTATG | 49. |

### Behavioral Testing

Blinding in these experiments at this stage was not possible, randomization was not performed. Littermates were used as control. The mice were kept in strict 12 h light/dark cycles. All behavior analysis was performed during the light cycle in a dedicated behavior room, where mice where acclimatized for at least 20 min before the experiment. All mice were naïve to the test. Behavior tests were performed with the Mecp2 deletion mice at 7 weeks of age, with the Xist deletion mice at 1 year of age.

### Open Field Test

The behavior of a mouse placed in a box with transparent walls is observed, which allows to assay general locomotor activity and anxiety. Individual mice were placed in the corner of a commercial open field activity arena (27×27 cm, Med Associates Inc.) which consists of a lit open area equipped with infrared beams on the side to track movements in x-y and z and allowed to move freely for 1 h, divided in blocks of 15 min. Total distance traveled, ambulatory time, ambulatory counts, stereotypy time, stereotypy counts, resting time, vertical counts, vertical time, zone entries, zone time, jump counts, jump time, average velocity, and ambulatory episodes were recorded and analyzed with automated software for each test mouse throughout the 60 min. test session. The distance traveled provides a measure of general activity and amount of time spent in the center (middle 20×20 cm) versus the edges of the arena, where the mouse feels more comfortable shielded by the walls measures anxiety.

### Rotarod Test

The mice were placed on top of a beam in a commercial rotorod apparatus (Ugo Basile) facing away from the experimenter's view. The beam was rotated such that forward locomotion is necessary to avoid falling off the beam. The rotorod is accelerated gradually from 4 to 40 rpm over a 5 min trial. One daily session of 3 trials with a minimal 15 min interval were conducted. Sessions were repeated for 3 days (total of 9 trials). If the mouse clings to the rod without moving (passive rotation) for two complete revolutions, it is considered to have fallen.

### Elevated Plus Maze Test

In this assay, mice are put in a plus-shaped maze (Med Associates) that has 4 alternating open and closed (walled) arms arranged perpendicularly and is elevated approximately 50 cm above the floor. The test is based on the innate drive of mice to explore novel environments while avoiding exposed, bright and unprotected environments. Each mouse was placed in the center hub of the maze (where the 4 arms meet) with its nose pointing inside a closed arm. Movement was recorded using a video tracking system for 10 minutes. The latency to first entry into an open arm and the time spent in the closed arms (measures of anxiety-like behavior), as well as total number of arm entries (open and closed, an indicator of hyperactivity), is recorded. Increased latency to enter the open arms, or increased time spent in the closed arms, indicates increased anxiety-like behavior.

### Aza Three Pulse Treatment

Aza was administered to the Xist2lox, Nestin-Cre F2 generation, by IP injection at 5 weeks old. Three injections 100 ul per 10 g of 0.033 mg/ml in sterile saline (or just sterile saline as control) were given over the course of a week (each injection separated by 2 days). Both Xist2lox/2lox and Xist Δ/Δ were injected and were randomly assigned to the treatment group. No specific randomization protocol was followed. RNA from the brain and liver were harvested (as described before) at 7 weeks of age (2 weeks after the first injection).

### Tissue Sectioning

The tissue was imbedded in TOC and frozen in a slurry of dry ice with isopentane. The obtained blocks were sliced at 8 micron with a cryostat.

### Fluorescence In Situ Hybridization (FISH)

A tissue section was immobilized on a glass slide, rinsed in cold PBS (5 min), pre-extracted in 0.5% CSKT on ice (6 min), fixed with 4% paraformaldehyde in PBS at room temperature (10 min) and then stored or washed in 70% EtOH. For hybridization the slide was dehydrated through sequential washing in 80%, 90% and 100% EtOH (2 min) and air-drying. DNA probe (Alexa 647-labeled oligonucleotide probes as described before) (Sunwoo H, Wu J Y, Lee J T (2015) The Xist RNA-PRC2 complex at 20-nm resolution reveals a low Xist stoichiometry and suggests a hit-and-run mechanism in mouse cells. Proc Natl Acad Sci USA 112:E4216-E4225) was then added to the slide, which was covered and incubated for 5 h at 37° C. After incubation the slide was washed 3 times with 50% formamide/2×SSC pH7.4 at 45° C. (5 min), 3 times with 0.5×SSC at 45° C. (5 min) and air-dried. The slide was then mounted with dapi containing antifade Vectashield (Vector Laboratories) and viewed under a Nikon Eclipse 90i microscope and Hamamatsu CCD camera. Image analysis (automated contrast enhancement for each channel in the whole image) was performed using Velocity (Perkin-Elmer).

### Statistics

Unless stated otherwise, error bars represent the standard error on the mean. For comparing 2 groups, p-values were calculated with the two sided T-test with equal variance. Variance was checked to be indeed equal with Levine's test and normality of the data was checked by looking at its representation in a histogram, its Q-Q plot and performing 2 tests of normality: Kolmogorov-Smirnov and Shapiro-Wilk. When this pointed out a possibility of non-normal distribution, the Mann-Whitney U test was performed. For comparing more than 2 groups one-way ANOVA test was performed. In case of unequal sample size, variance or a non-normal distribution, the Brown Forsythe test was performed. In the cumulative density plots p values were calculated using the Wilcoxon rank sum test.

### Data Availability

RNA-seq data was deposited to the Gene Expression Omnibus (GEO) under accession number GSE97077.

### Example 1. Pharmacological Synergy Through a Mixed Modality Approach

While the pharmaceutical industry has focused almost exclusively on targeting proteins, long noncoding RNAs (lncRNA) have become increasingly attractive as pharmacological targets (Matsui M and Corey D R. Non-coding RNAs as drug targets. Nat Rev Drug Discov. 2017; 16:167-179). With improving ASO technology, lncRNAs are now also pharmacologically accessible. ASOs are high molecular weight compounds that have been optimized over the past 50 years through chemical modifications to acquire greater stability, selectivity, and bioavailability (Bennett C F and Swayze E E. RNA targeting therapeutics: Molecular mechanisms of antisense oligonucleotides as a therapeutic platform. Annu Rev Pharmacol Toxicol. 2010; 50:259-293; Southwell A L et al. Antisense oligonucleotide therapeutics for inherited neurodegenerative diseases. Trends Mol Med. 2012; 18:634-643). Since ASOs bind their target through Watson-Crick basepairing interactions, they can be rationally designed and hit previously “undruggable” targets. Notably, ASO technology has achieved success in treating hypercholesterolemia (Kynamro™) and spinal muscular atrophy (Spinraza™).

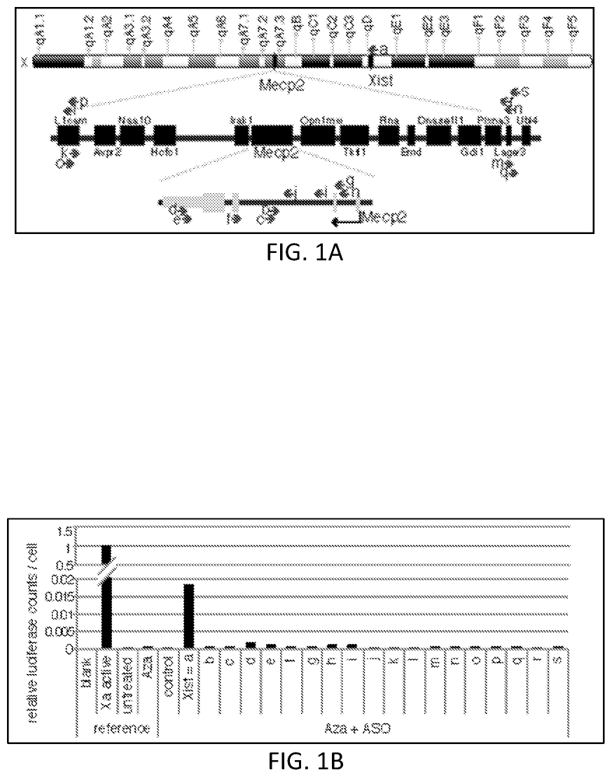

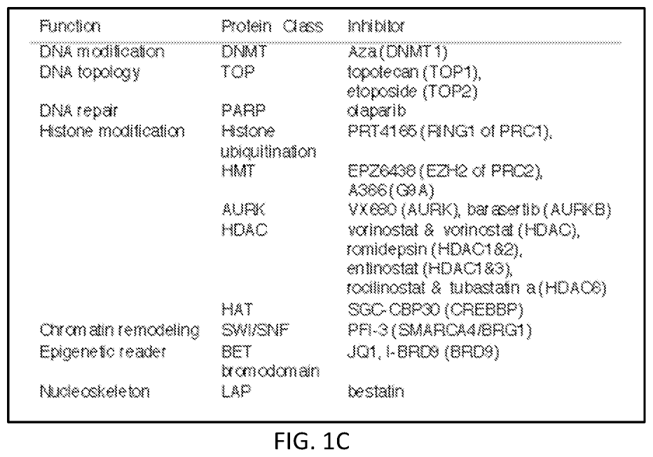

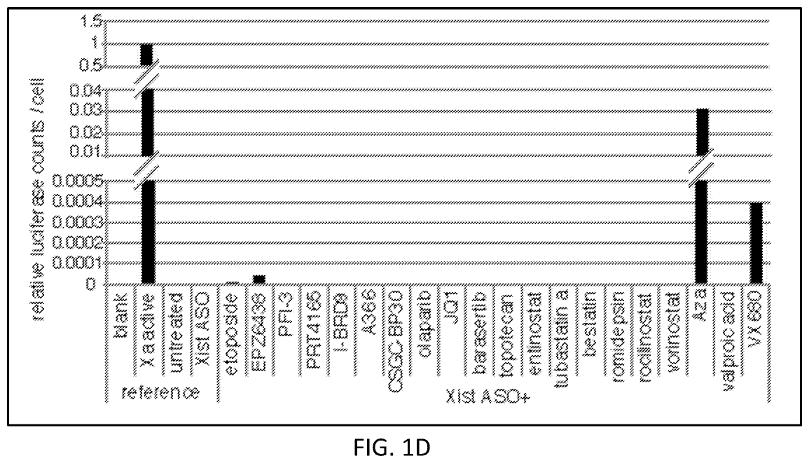

We asked whether an ASO could also be developed for Xi-reactivation. We screened a small ASO library against various targets of potential interest, including Xist RNA and an antisense transcript to Mecp2 (Mecp2-as) (FIG. 1A, FIG. 6, Table S-I). In designing the ASOs, we chose phosporothioate backbone and locked nucleic acid (LNA™) chemistry (Wahlestedt C et al. Potent and nontoxic antisense oligonucleotides containing locked nucleic acids. Proc Natl Acad Sci USA. 2000; 97:5633-5638) for its in vivo and in vitro stability, and increased affinity and selectivity for RNA targets. All were designed as gapmers, with unmodified deoxyribonucleosides in the center flanked by 5′ and 3′ terminal locked nucleosides, to direct RNAse-H-mediated cleavage of the target transcript. We tested each ASO on an immortalized clonal mouse fibroblast cell line carrying an Mecp2:Luciferase knock-in reporter on the Xi (Sripathy S et al. Screen for reactivation of MeCP2 on the inactive X chromosome identifies the BMP/TGF-β superfamily as a regulator of XIST expression. Proc Natl Acad Sci USA. 2017; 114:1619-1624; Lessing D et al. A high-throughput small molecule screen identifies synergism between DNA methylation and Aurora kinase pathways for X reactivation. Proc Natl Acad Sci USA. 2016; 113:14366-14371). The luciferase reporter provides a highly sensitive enzymatic detection method with a large dynamic range. Because previous studies provide strong support for synergistic Xi-reactivation (Csankovszki G et al. Synergism of Xist RNA, DNA methylation, and histone hypoacetylation in maintaining X chromosome inactivation. J Cell Biol. 2001; 153:773-784; Lessing D et al. A high-throughput small molecule screen identifies synergism between DNA methylation and Aurora kinase pathways for X reactivation. Proc Natl Acad Sci USA. 2016; 113: 14366-14371; Minkovsky A et al. A high-throughput screen of inactive X chromosome reactivation identifies the enhancement of DNA demethylation by 5-aza-2′-dC upon inhibition of ribonucleotide reductase. Epigenetics Chromatin. 2015; 8:42; Minajigi A, et al. A comprehensive Xist interactome reveals cohesin repulsion and an RNA-directed chromosome conformation. Science. 2015; 349: aab2276-12), we examined the efficacy of each ASO in the presence of 0.5 uM decitabine (“Aza”; 5-aza-2′-deoxycytidine) for three days. Notably, Aza combinations with ASOs against Mecp2-as or various nearby ASOs yielded inconsistent, low, or no Mecp2:Luciferase reactivation relative to untreated samples or Aza-only samples. Remarkably, however, the Xist ASO+Aza combination showed a robust, reproducible response equivalent to 3% of normal MECP2 levels on the Xa (FIG. 1B, FIG. 6). These data suggest that targeting Xist RNA together with DNA methylation may be an effective method of achieving partial Xi-reactivation.

Next, we performed the reciprocal analysis and asked whether combining the Xist ASO with small molecule inhibitors of other epigenetic pathways may be efficacious. We tested commercially available compounds for factors identified in an Xist proteomic study (Minajigi A, et al. A comprehensive Xist interactome reveals cohesin repulsion and an RNA-directed chromosome conformation. Science. 2015; 349: aab2276-12) (FIG. 1C; Table S-II). In combination with the Xist ASO, inhibitors of EZH2 (EPZ6438), and Aurora kinase (VX680) showed varying degrees of upregulation (FIG. 1D). These inhibitors were previously identified as potential Xi-reactivators in independent screens (Bhatnagar S et al. Genetic and pharmacological reactivation of the mammalian inactive X chromosome. Proc Natl Acad Sci USA. 2014; 111:12591-12598; Lessing D et al. A high-throughput small molecule screen identifies synergism between DNA methylation and Aurora kinase pathways for X reactivation. Proc Natl Acad Sci USA. 2016; 113:14366-14371). Intriguingly, none of the inhibitors against recently identified targets demonstrated an efficacy that rivaled Aza+Xist ASO (FIG. 1D), though we limited testing to Xist interactors for which small molecule probes were available. Thus, in reciprocal tests of ASOs and small molecule inhibitors, Xist ASO+Aza emerged as the top candidate. This mixed modality combination yielded a level of reactivation not previously seen. Henceforth, we focus on characterization of this combination.

### Example 2. The Xist ASO+Aza Synergistic Duo

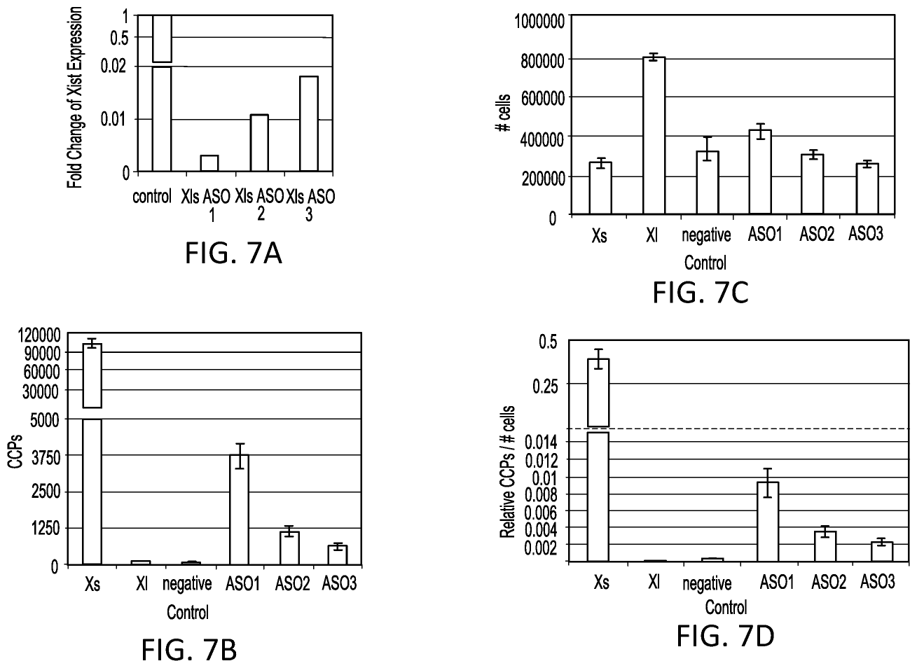

To exclude off-target effects, we created three Xist gapmers (1, 2, and 3) that target different regions of exon 1 (FIG. 2A). Introduction of any single Xist ASO at 20 nM by Lipofectamine transfection resulted in >95% Xist depletion in mouse embryonic fibroblasts (MEFs) for 3-5 days (FIG. 2B, FIG. 7A) To test the Xist ASO+Aza combinations and look for potential Xi-reactivation of Mecp2, we used the cell line carrying the Mecp2:luciferase reporter on the Xi. We examined 5 different Aza concentrations given as a single dose on day 0 against a fixed 20 nM concentration of the Xist (Gapmer 1 was selected for further studies) or control (Scr) ASO and examined cells over a 3-day treatment period. Whereas Aza concentrations between 0 to 0.5 uM were tolerated, higher concentrations (1.0, 2.5 uM) resulted in increased cell death (FIG. 2C). At 20 nM, the Xist ASO was not toxic relative to the control ASO (FIG. 2C, compare top left versus bottom left panels). These data suggest that the combination of 20 nM ASO and a single pulse of 0.5 uM Aza (its IC50), would be well-tolerated by MEF cells in culture. Notably, an Aza pulse was also used to prime cells in a small molecule screen (Lessing D, et al. A high-throughput small molecule screen identifies synergism between DNA methylation and Aurora kinase pathways for X reactivation. Proc Natl Acad Sci USA. 2016; 113:14366-14371).

After three days of treatment (FIG. 2D), neither the control Scr ASO (20 nM) nor Xist ASO (20 nM gapmer 1) resulted in measurable luminescent counts per second (LCPS). Application of Aza (0.5 uM) by itself caused the previously reported baseline level of Mecp2:luciferase reactivation (Bhatnagar S, et al. Genetic and pharmacological reactivation of the mammalian inactive X chromosome. Proc Natl Acad Sci USA. 2014; 111:12591-12598; Lessing D et al. A high-throughput small molecule screen identifies synergism between DNA methylation and Aurora kinase pathways for X reactivation. Proc Natl Acad Sci USA. 2016; 113:14366-14371; Minkovsky A et al. A high-throughput screen of inactive X chromosome reactivation identifies the enhancement of DNA demethylation by 5-aza-2′-dC upon inhibition of ribonucleotide reductase. Epigenetics Chromatin. 2015; 8:42). On the other hand, combining this Xist ASO with Aza resulted in a significant synergistic increase, in accordance with the in vivo data. This level of increase was equivalent to 1.8% of the theoretical maximum—i.e., ˜2% of the protein level of Mecp2:luciferase when it was carried on the expressed Xa. This is equivalent to a 12,000-fold increase in Xi-Mecp2 expression and is considerably greater than the 600× upregulation observed in a previous screen (Lessing D, et al. A high-throughput small molecule screen identifies synergism between DNA methylation and Aurora kinase pathways for X reactivation. Proc Natl Acad Sci USA. 2016; 113:14366-14371). When cells were treated for 5 days with the Xist ASO+Aza combination, Mecp2:luciferase upregulation increased to as much as 2.0-3.5% (average 2.5%, n=3; FIG. 2E) or up to 30,000-fold of Xi levels. Single treatments with the ASO or Aza remained significantly lower. To exclude off-target effects, 2 other Xist gapmers (gapmers 2,3; FIG. 2A) were tested and were found to also upregulate MECP2 (FIG. 7B-D).

### Example 3. Transcriptomic Analysis Indicates Selective Xi-Reactivation

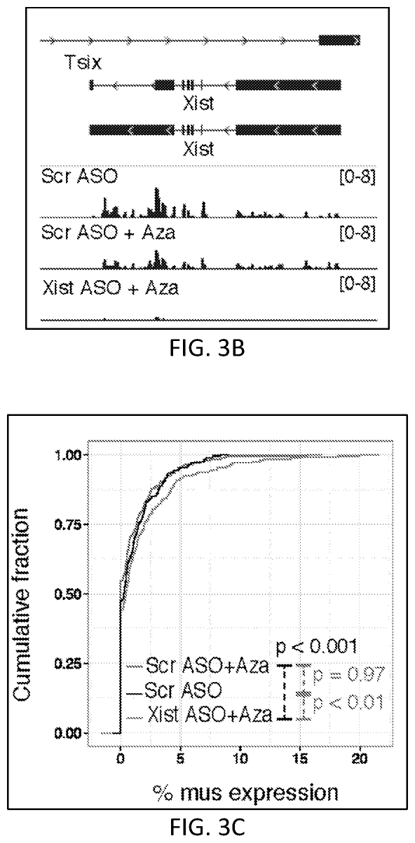

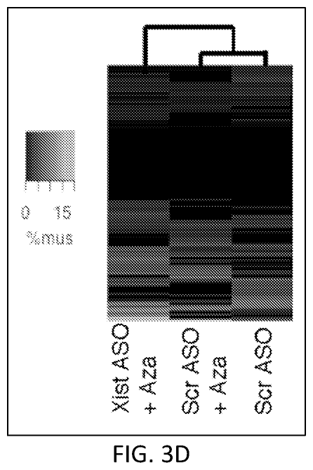

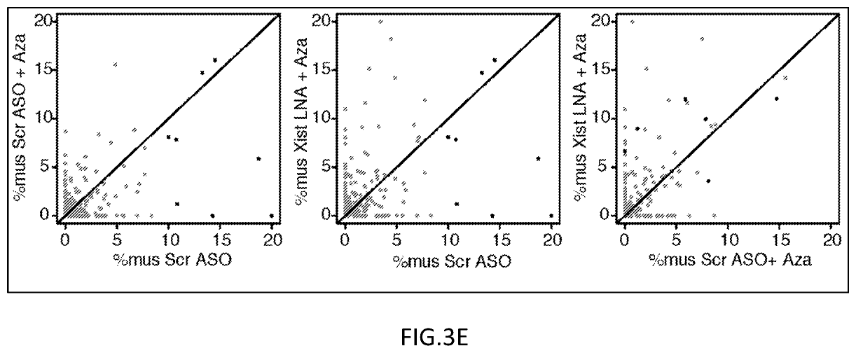

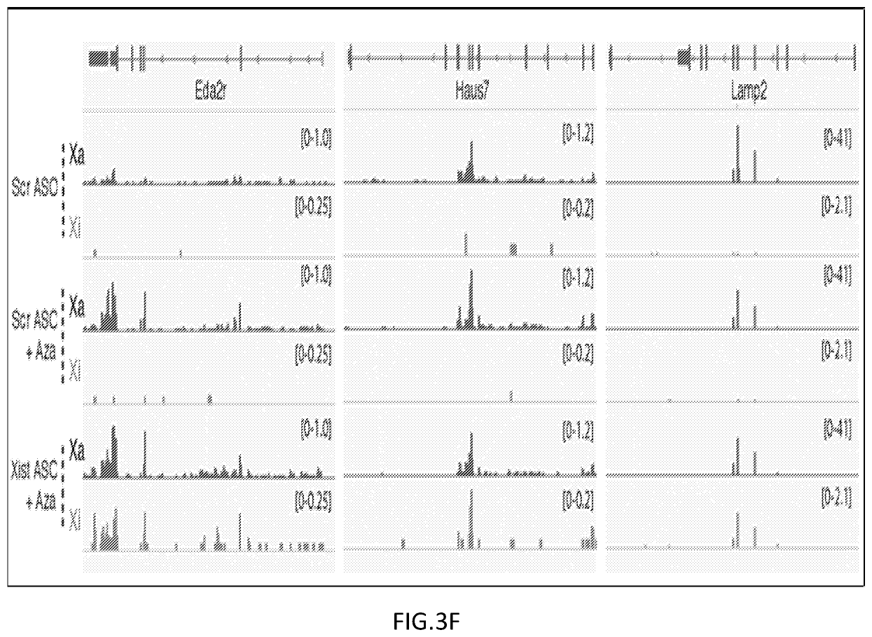

We asked if the Xist ASO+Aza combination achieved effects on the Xi beyond Mecp2 reactivation. The Xi-reactivation strategy would have the potential to treat a number of X-linked diseases, including those caused by mutations of CDKL5, KIAA2022, USP9X, SMC1a, HDAC8, and FMR1. We tested the Xist ASO+Aza combination on an F1 hybrid fibroblast line in which the Xi is of Mus musculus (mus) strain origin and the Xa of Mus casteneus (cas) origin (Yildirim E et al. X-chromosome hyperactivation in mammals via nonlinear relationships between chromatin states and transcription. Nat Struct Mol Biol. 2011; 19:56-61). Between the X^{mus} and X^{cas}, there are over 600,000 X-linked sequence polymorphisms that enable determination of allelic origin (Pinter S F et al. Spreading of X chromosome inactivation via a hierarchy of defined Polycomb stations. Genome Res. 2012; 22:1864-1876). We established an allele-specific pipeline for RNA-seq analysis (FIG. 3A). Among 1063 X-linked genes, only 510 were expressed (FPKM>0) in the fibroblast line. Among these, we considered only the 315 genes with total allelic reads>12. Of these, 243 were considered to be subject to XCI, with a mus-fraction<1/11. RNA-seq analysis showed that Xist^{mus} expression from the Xi was knocked down by the ASO to nearly undetectable levels (FIG. 3B). Xist^{cas} was not expressed from the Xa. As a result, cumulative density plot (CDP) analysis of X-gene expression showed a significant right shift in Xi expression (allelic reads of Xi/Xi+Xa) when cells were treated with Xist ASO+Aza combination in comparison to both the Scr ASO treatment and the Scr ASO+Aza treatment (FIG. 3C). Heatmap (FIG. 3D) and scatterplot (FIG. 3E) analyses revealed a substantial number of Xi-reactivated genes in the Xist ASO+Aza treated samples relative to treatment with Scr ASO and Aza. Specific genic examples also demonstrated the extent of Xi reactivation seen specifically in the combination treatment (FIG. 3F). RNA-seq did not offer enough sensitivity to see reactivation of Mecp2, especially in fibroblasts, where Mecp2 is not expressed as highly as in neurons (Mecp2 is not fused to Luciferase in the hybrid cell line). Unlike the luciferase assay, a 2-5% increase in RNA-seq reads (FPKM) is generally difficult to distinguish from noise. But, taken together, these data show a selective reactivation of the Xi relative to the Xa and the rest of the genome. They highlight the potential for treating other diseases and affirmed the idea of pharmacological synergy between depleting Xist RNA and treating with Aza.

### Example 4. Female Mice Lacking Xist in the Brain Live a Normal Lifespan without Reduced Fitness

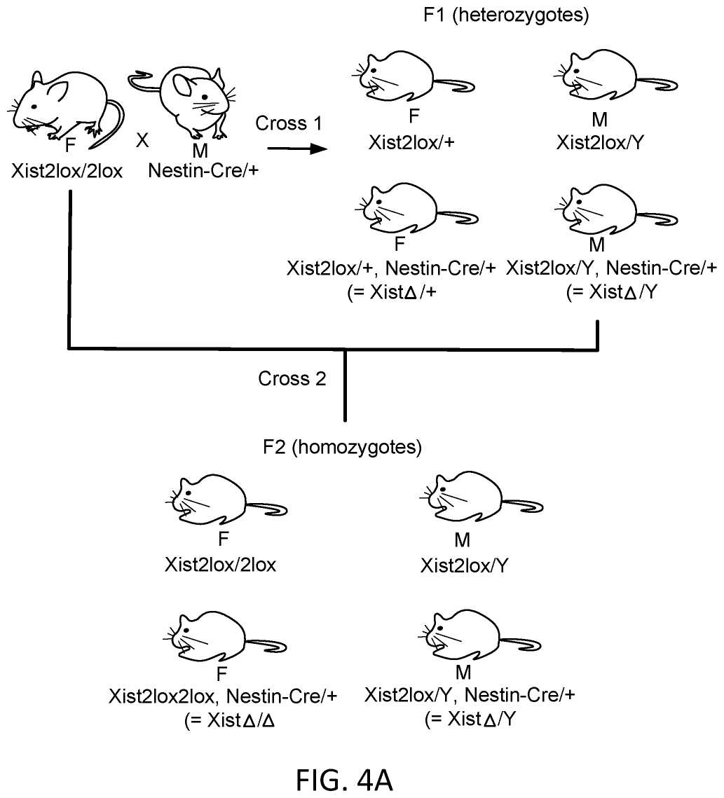

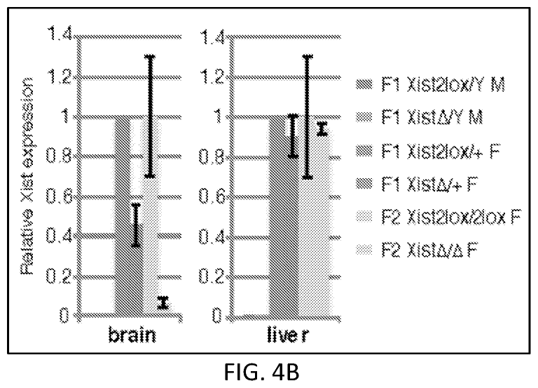

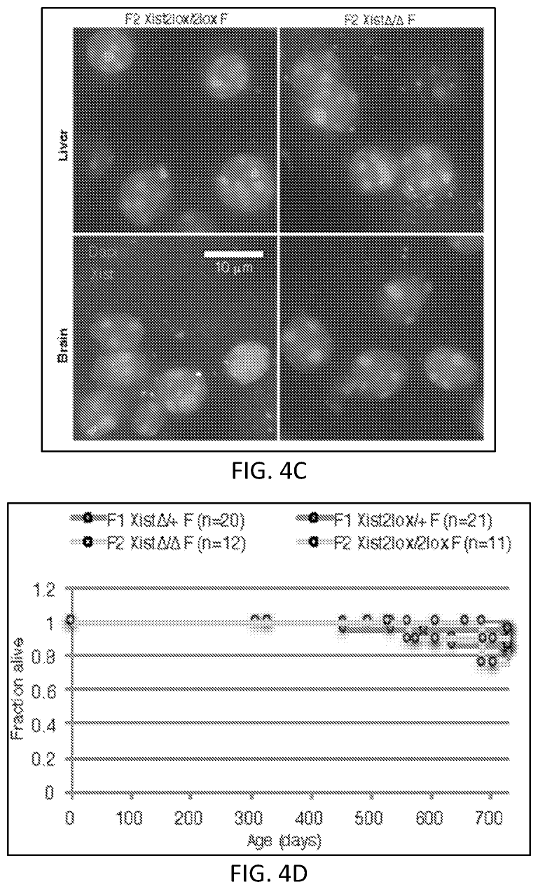

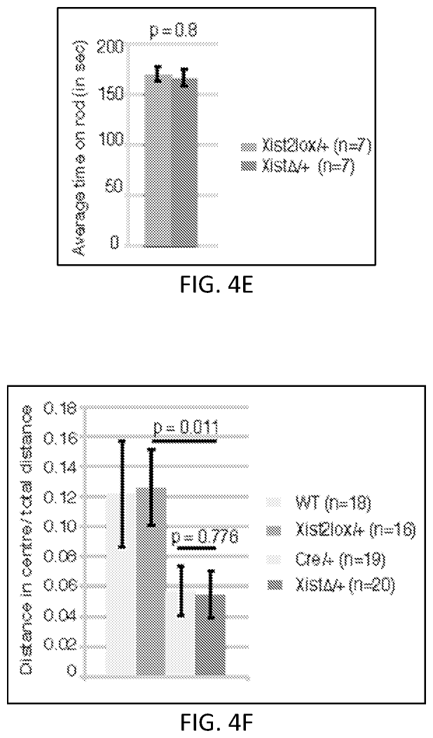

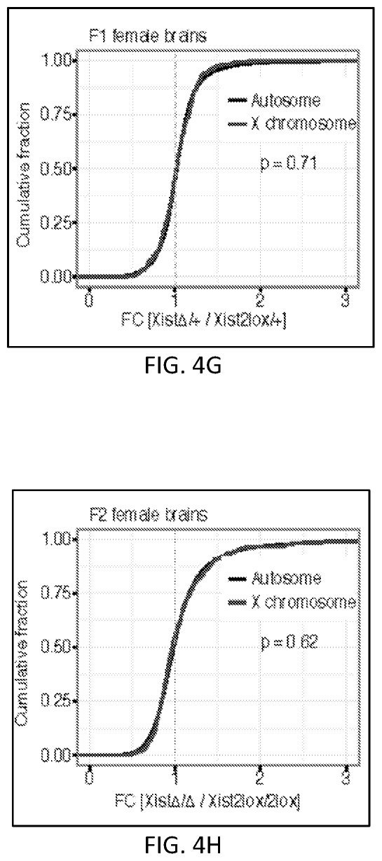

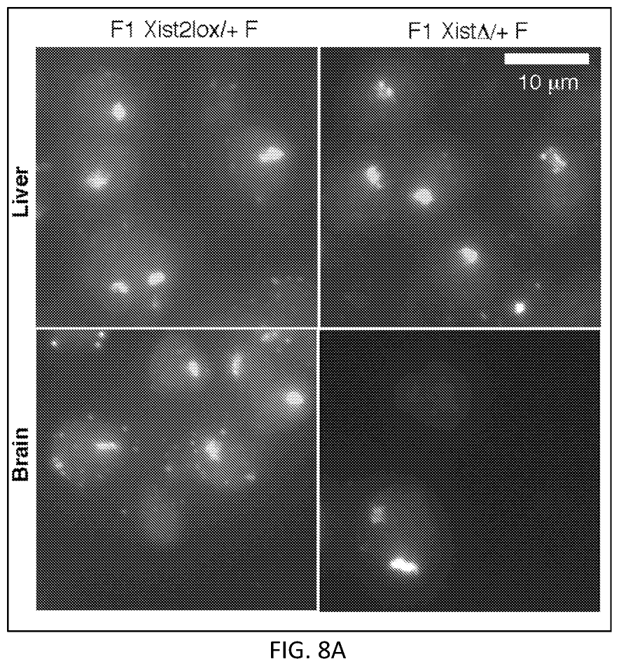

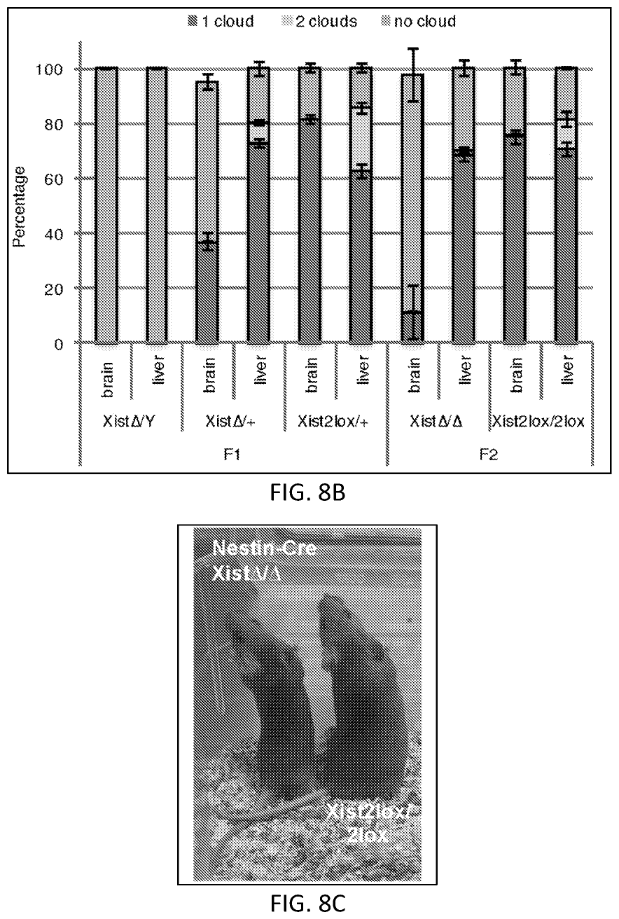

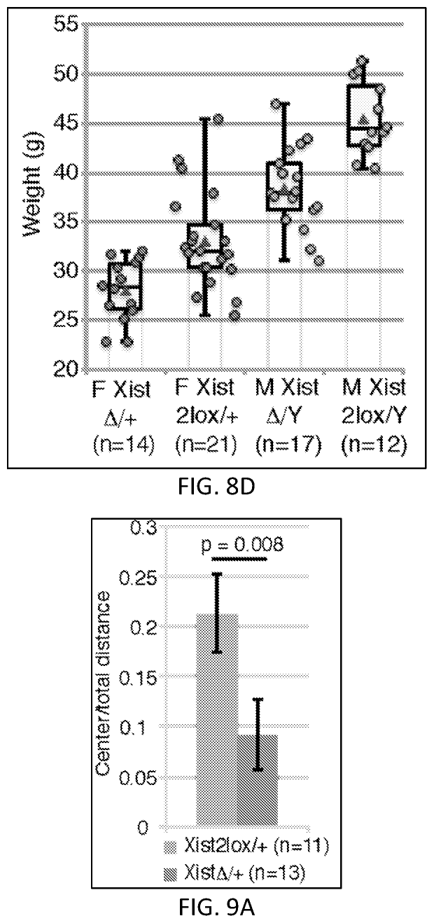

In view of the reduced fitness of the mice lacking Xist RNA (Yang L et al. Female mice lacking Xist RNA show partial dosage compensation and survive to term. Genes Dev. 2016; 30:1747-1760; Yildirim E et al. Xist RNA is a potent suppressor of hematologic cancer in mice. Cell. 2013; 152:727-742), concerns might be raised for any treatment involving Xist depletion. Therefore, we next explored whether Xist loss and associated X-chromosome dosage change could be tolerated in the brain, the target organ of various X-linked neurodevelopmental disorders, including RTT, CDKL5, and Fragile X Syndromes. Using a Nestin-Cre driver (Tronche F et al. Disruption of the glucocorticoid receptor gene in the nervous system results in reduced anxiety. Nat Genet. 1999; 23:99-103), we conditionally knocked out Xist in embryonic brain cells at embryonic day 11 (E11)—a developmental stage long after establishment of XCI (FIG. 4A). Our cross resulted in heterozygous F1 females in whom Xist was deleted from the Xi in half of all neuronal cells. We then generated homozygously deleted F2 mice by backcrossing F1 XistΔ/Y, Nestin-Cre male to female Xist2lox/2lox mice. We confirmed the deletions by RNA FISH and RT-qPCR for Xist expression. In F1 heterozygous females, the number of Xist RNA foci was reduced by half in the brain (FIG. 8A, FIG. 8B), as were relative total Xist levels (FIG. 4B). Xist loss is evident in some cells of the XistΔ/+ brain (36% 1 cloud, 64% no cloud and 0% 2 clouds with n=207 for F1 XistΔ/+F brain, 86% 1 cloud, 15% no cloud and 4% 2 clouds with n=226 for F1 XistΔ/+F liver, 76% 1 cloud, 24% no cloud and 0% 2 clouds with n=206 for F1 Xist2lox/+F brain, 66% 1 cloud, 17% no cloud and 17% 2 clouds with n=202 for F1 Xist2lox/+F liver (FIG. 8A). In F2 homozygous females, Xist expression was absent in the brain (FIG. 4B, FIG. 4C). Xist loss is evident in all cells of the XistΔ/Δ brain (3% with one cloud, 97% with no cloud and 0% with two clouds, with n=367 for F2 XistΔ/Δ brain F; 69% with one cloud, 30% with no cloud, and 1% with two clouds, with n=411 for F2 XistΔ/Δ F liver; 75% with one cloud, 24% with no cloud, and 1% with two clouds, with n=280 for F2 Xist2lox/2lox F brain; and 70% with one cloud, 19% with no cloud, and 11% with two clouds, with n=342 for F2 Xist2lox/2lox F liver (FIG. 4B). In the liver, where Nestin-Cre was not expressed, Xist expression was unaltered.

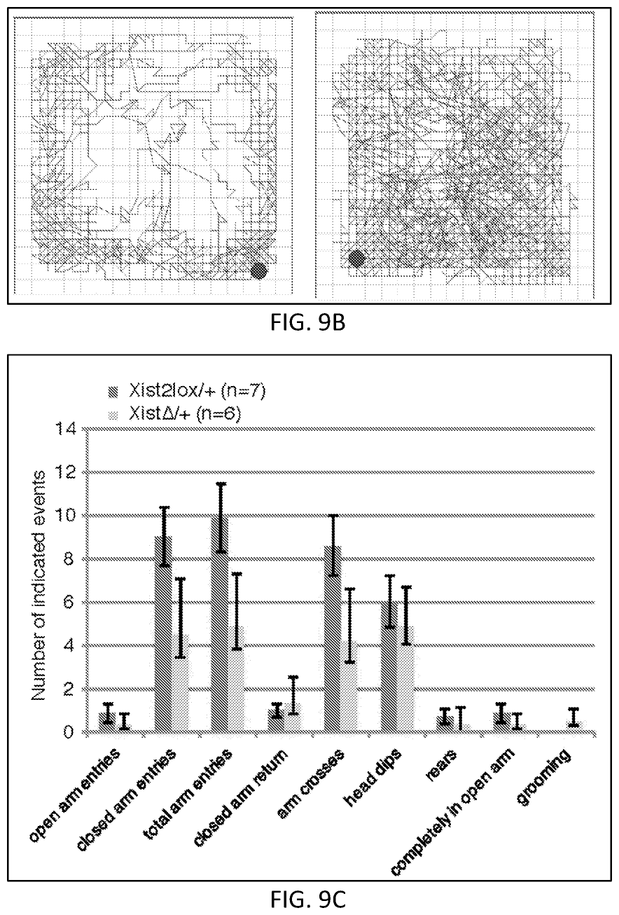

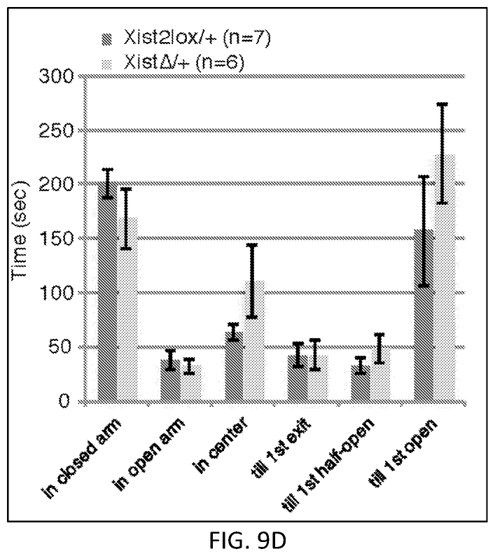

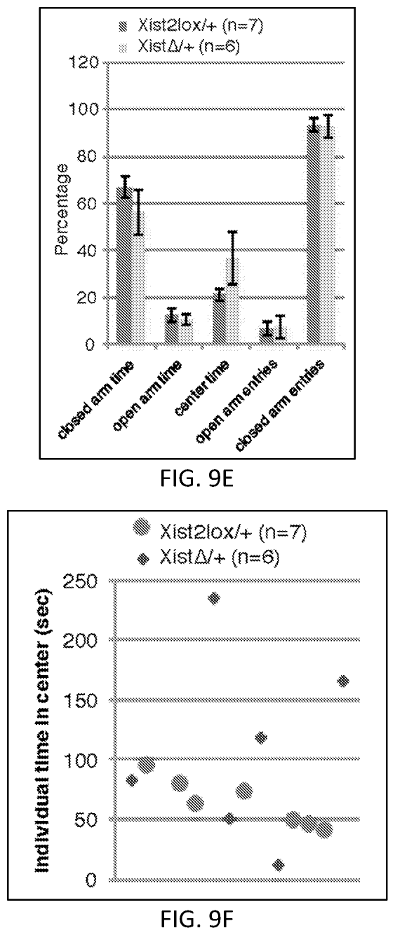

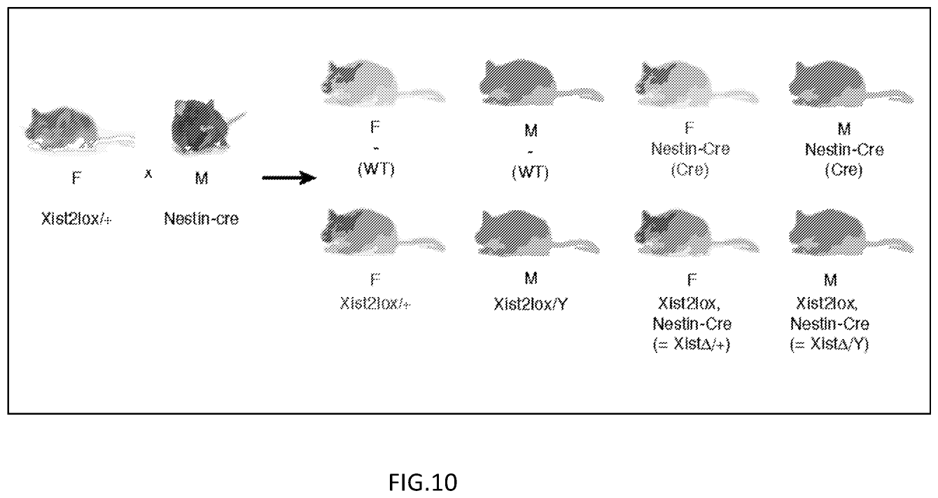

We then asked whether brain-specific Xist deletion resulted in an overt phenotype in mice. In contrast to mice bearing Xist deletions in blood cells and whole body (Yang L et al. Female mice lacking Xist RNA show partial dosage compensation and survive to term. Genes Dev. 2016; 30:1747-1760; Yildirim E et al. Xist RNA is a potent suppressor of hematologic cancer in mice. Cell. 2013; 152:727-742), both F1 and F2 Xist-mutant females were healthy and exhibited a lifespan similar to that of wild-type littermates (FIG. 4D). There was no difference in gait or mobility, as the mice showed equal performance on the rotarod (FIG. 4E). Some differences—such as in body weight—between mutant and wild-type mice were found, but these could be attributed exclusively to the Nestin-Cre knock-in (FIG. 8C, FIG. 8D) (Galichet C et al. Nestin-Cre mice are affected by hypopituitarism, which is not due to significant activity of the transgene in the pituitary gland. PLoS One. 2010; 5:e11443). Notably, Nestin-Cre males who should not be affected by an Xist deletion, nevertheless showed reduced size. The open field and elevated plus maze also showed differences (FIG. 9A-F). Because the cross as set up (FIG. 4A) rendered the Nestin-Cre allele and Xist deletions inseparable, the phenotype could be due to either the presence of Nestin-Cre (Giusti S A et al. Behavioral phenotyping of Nestin-Cre mice: Implications for genetic mouse models of psychiatric disorders. J Psychiatr Res. 2014; 55:87-95) or the absence of Xist. Through an additional cross between an Xist2lox/+ female and a Nestin-Cre male to separate the Nestin-Cre genotype from the Xist2lox genotype (FIG. 10), we attributed observed differences strictly to Nestin-Cre. Repeat open field testing revealed the same significant differences between Xist2lox versus Nestin-Cre (p<0.02), whereas the difference between XistΔ/+ versus Nestin-Cre was insignificant (p>0.78)(FIG. 4F). Because an intercross of F1 animals yielded F2 animals of non-uniform backgrounds, the F2 generation was not subjected to behavior testing.

Given minimal phenotypic differences, we performed RNA-sequencing (RNA-seq) analysis on the brains of F1 XistΔ/+ and F2 XistΔ/Δ females at 1 year of age and looked for deviation of X-linked and autosomal gene expression relative to brains of Xist2lox/2lox control females. Because the mice lacked allelic information that would allow distinguishing Xi from Xa expression, we analyzed composite (both alleles) gene activities on the X-chromosome and displayed transcriptomic data in CDPs for fold-changes (FC) between test and control brains (FIG. 4G, FIG. 4H) (Yang L et al. Female mice lacking Xist RNA show partial dosage compensation and survive to term. Genes Dev. 2016; 30:1747-1760). Interestingly, neither heterozygous nor homozygous mutant brains showed any overall change in X-linked gene expression relative to controls (red curves centered at FC=1 with little deviation). In addition, there was no significant deviation of X-linked gene expression relative to autosomal expression, as shown by the nearly superimposable red and black curves (FIG. 4G, FIG. 4H). Thus, unlike in the blood (Yildirim E et al. Xist RNA is a potent suppressor of hematologic cancer in mice. Cell. 2013; 152:727-742), the Xi in the brain appears to be relatively stable when Xist is conditionally deleted.

### Example 5. Modeling Pharmacological Intervention in the Xist-Deleted Mouse

To assess whether Aza could synergize with the Xist deletion to destabilize the Xi in the brain, we treated XistΔ/Δ female mice at 5 weeks of age—the approximate age at which Rett phenotypes are clearly manifested. Cognizant of cytotoxic effects of long-term Aza treatment (Momparler R L et al. Pilot phase I-II study on 5-aza-2′-deoxycytidine (Decitabine) in patients with metastatic lung cancer. Anticancer Drugs. 1997; 8:358-368), we tested a short-term treatment on the principle that DNA methylation states are stably propagated through mitotic divisions (Zaidi S K et al. Architectural epigenetics: Mitotic retention of mammalian transcriptional regulatory information. Mol Cell Biol. 2010; 30:4758-4766; Kordasiewicz H B, et al. Sustained therapeutic reversal of Huntington's disease by transient repression of huntingtin synthesis. Neuron. 2012; 74:1031-1044). Because Aza can cross the blood-brain-barrier (Chabot G G et al. Plasma and cerebrospinal fluid pharmacokinetics of 5-Aza-2′-deoxycytidine in rabbits and dogs. Cancer Res. 1983; 43:592-597), we administered three sequential Aza pulses via intraperitoneal (IP) injections over the course of one week and then followed health over time.

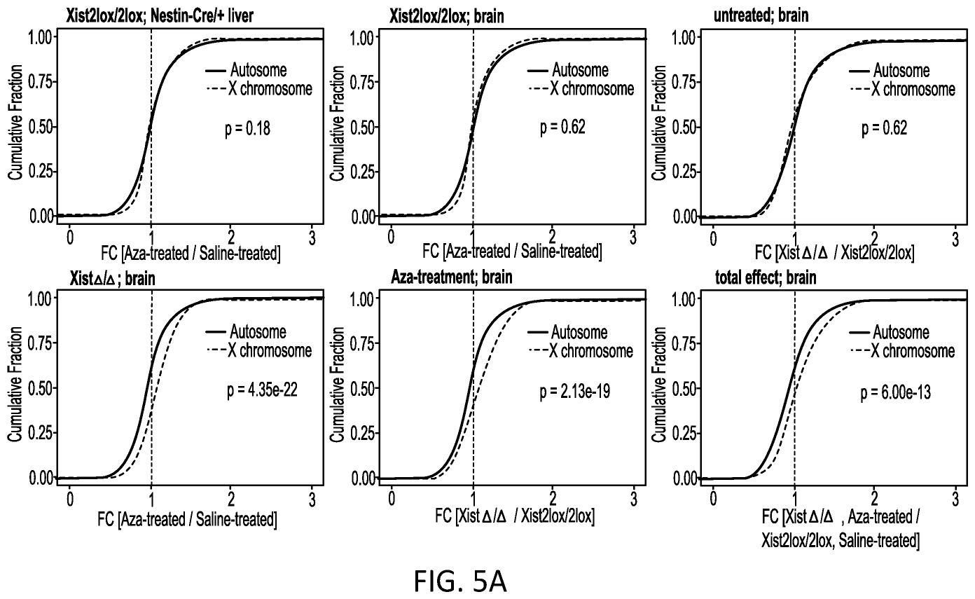

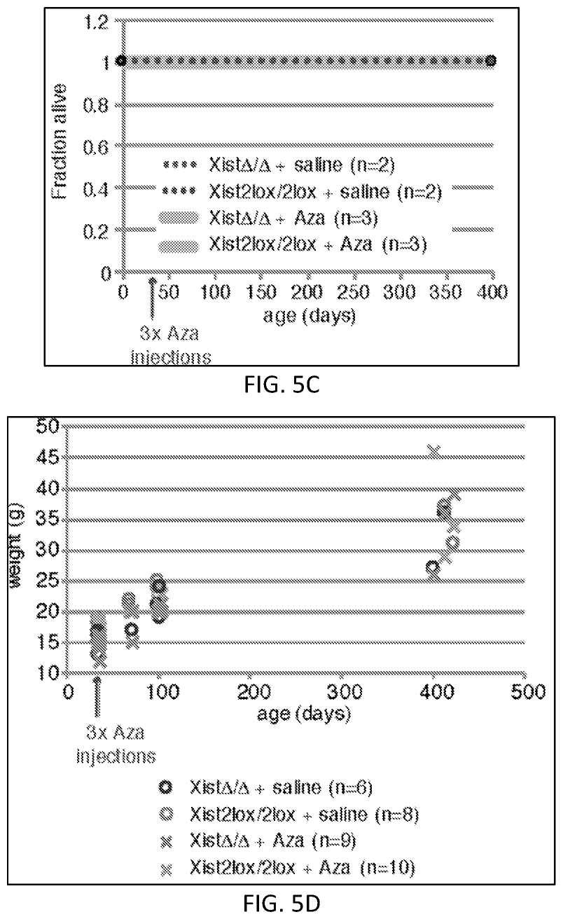

To examine potential changes in X-linked gene expression, we performed transcriptomic analysis on brain (target organ) and liver (non-targeted, control organ) harvested from a subset of mice at two weeks after drug treatment. First, in the liver where Xist was not deleted by the neuronal-specific Nestin-Cre driver, X- and autosomal gene expression remained balanced, even after treatment with Aza (FIG. 5A, upper left graph). Evidently, the three-pulse Aza treatment did not result in a net upward change of gene expression on either the X or autosomes. Second, Aza treatment also did not result in X-to-autosomal gene imbalance in the Xist2lox/2lox (wild-type) brain (FIG. 5A, upper middle graph). Three-pulse IP injections of Aza therefore did not result in global changes in X-linked or autosomal gene expression in wild-type tissues. Furthermore, as shown above, deleting Xist by itself did not result in significant gene expression changes (FIG. 4G; 5A, upper right graph). Thus, neither deleting Xist alone nor short-term treatment with Aza alone was sufficient to perturb X-linked gene expression. On the other hand, combining the Xist deletion with pulse Aza treatment resulted in highly significant positive changes in X-linked gene expression relative to autosomal expression. A right shift of the X-linked curve could be observed when comparing Aza- versus saline-treated XistΔ/Δ brain (FIG. 5A, bottom left graph), indicating that Aza treatment is necessary to unmask the effect of the Xist deletion in brain. Conversely, a right shift was also observed when comparing XistΔ/Δ brain to control brain after Aza-treatment (FIG. 5A, bottom middle graph), indicating that the Xist deletion is required to reveal the effect of Aza in the brain. Indeed, while neither deleting Xist nor Aza treatment alone led to significant changes (FIG. 5A, top middle and top right), combining the Xist deletion and Aza administration yielded increased X-gene expression relative to autosomes (FIG. 5A, bottom right graph).

Notably, a short-term pulse treatment of Aza administered systemically (IP) was sufficient to unmask the effect of the Xist deletion across the blood-brain-barrier, with X-upregulation evident in the brain two weeks later. This is a potentially promising finding, given that Aza can have considerable toxic effects if administered continuously over time (Momparler R L et al. Pilot phase I-II study on 5-aza-2′-deoxycytidine (Decitabine) in patients with metastatic lung cancer. Anticancer Drugs. 1997; 8:358-368). To determine if the short-term pulse treatment resulted in untoward long-term consequences, we followed treated animals over one year and noted no measurable differences in health and lifespan (FIG. 5B). Indeed, all mice advanced to 1-2 year of age. At the time of treatment, the body weight of Xist2lox/2lox mice was on average higher than of XistΔ/Δ due to the Nestin-Cre background. The treatment did not introduce significant differences between the weight of mice from the Aza and saline treatment groups (FIG. 5C). We conclude that the combination of Xist loss and short-term Aza treatment in vivo leads to a partial upregulation of the X-chromosome that is tolerated in vivo, at least during the short-term period of testing.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition comprising an inhibitor of Xist RNA and an inhibitor of an Xist-interacting protein.

2. A method of activating an inactive X-linked allele in a cell, preferably a cell of a female heterozygous subject or male hemizygous subject, the method comprising administering to the cell an inhibitor of Xist RNA and an inhibitor of an Xist-interacting protein.

3. A method of activating an epigenetically silenced or hypomorphic allele on the active X-chromosome in a cell, preferably in a cell of a male or female hemizygous or heterozygous subject, the method comprising administering to the cell an inhibitor of Xist RNA and an inhibitor of an Xist-interacting protein.

4. The composition of claim 1 or the method of claim 2 or 3, wherein the inhibitor of XIST RNA is an inhibitory nucleic acid targeting XIST lncRNA or a gene encoding XIST lncRNA.

5. The composition of claim 1 or the method of claim 2 or 3, wherein the inhibitor of an Xist-interacting protein inhibits a protein shown in Table 1, preferably including proteins selected from the group consisting of: SMC1a; SMC3; WAPL; RAD21; KIF4; PDS5a/b; CTCF; TOP1; TOP2a; TOP2b; SMARCA4 (BRG1); SMARCA5; SMARCC1; SMARCC2; SMARCB1; CBX7; RING1a/b (PRC1); PRC2 (EZH2, SUZ12, RBBP7, RBBP4, EED); AURKB; SPEN/MINT/SHARP; DNMT1; SmcHD1; CTCF; MYEF2; ELAV1; SUN2; Lamin-B Receptor (LBR); LAP; hnRPU/SAF-A; hnRPK; hnRPC; PTBP2; RALY; MATRIN3; MacroH2A; and ATRX.

6. The composition or method of claims 1-3, wherein the inhibitor of an Xist-interacting protein is a small molecule inhibitor or an inhibitory nucleic acid that targets a gene encoding the Xist-interacting protein.

7. The composition or method of claims 1-3, wherein the inactive X-linked allele is associated with an X-linked disorder, and the inhibitor of Xist RNA or an Xist-interacting protein are administered in a therapeutically effective amount.

8. The method of claim 2, wherein the active X-linked allele is associated with an X-linked disorder, and the inhibitor of Xist RNA or an Xist-interacting protein are administered in a therapeutically effective amount.

9. The method of claim 2-3 or 8, wherein the cell is in a living subject.

10. The method of claim 2-3 or 8, further comprising administering one or more of:
(a) an inhibitory nucleic acid targeting a strong or moderate RNA-binding protein binding site on the X chromosome; and/or
(b) an inhibitory nucleic acid targeting a suppressive RNA (supRNA) associated with the X-linked allele.

11. The composition of claim 1, further comprising administering one or more of:
(a) an inhibitory nucleic acid targeting a strong or moderate RNA-binding protein binding site on the X chromosome; and/or
(b) an inhibitory nucleic acid targeting a supRNA associated with the X-linked allele.

12. The composition or method of claim 4, wherein the inhibitory nucleic acid is identical or complementary to at least 8 consecutive nucleotides of a strong or moderate binding site nucleotide sequence as set forth in Tables A, IVA-C, or XIII-XV of WO 2014/025887 or Table 1 of U.S. Ser. No. 62/010,342, or is complementary to at least 8 consecutive nucleotides of a supRNAs as set forth in Tables VI-IX or XVI-XVIII of WO 2014/025887.

13. The composition or method of claim 4, wherein the inhibitory nucleic acid does not comprise three or more consecutive guanosine nucleotides or does not comprise four or more consecutive guanosine nucleotides.

14. The composition or method of claim 4, wherein the inhibitory nucleic acid is 8 to 30 nucleotides in length.

15. The composition or method of claim 4, wherein at least one nucleotide of the inhibitory nucleic acid is a nucleotide analogue.

16. The composition or method of claim 4, wherein at least one nucleotide of the inhibitory nucleic acid comprises a 2′ O-methyl, optionally wherein each nucleotide of the inhibitory nucleic acid comprises a 2′ O-methyl.

17. The composition or method of claim 4, wherein the inhibitory nucleic acid comprises at least one ribonucleotide, at least one deoxyribonucleotide, or at least one bridged nucleotide.

18. The composition or method of claim 17, wherein the bridged nucleotide is a locked nucleic acid (LNA) nucleotide, a 2′-O-Ethyl (cEt) modified nucleotide or a 2′-O,4′-C-ethylene (ENA) modified nucleotide.

19. The composition or method of claim 4, wherein each nucleotide of the inhibitory nucleic acid is a LNA nucleotide.

20. The composition or method of claim 4, wherein one or more of the nucleotides of the inhibitory nucleic acid comprise 2′-fluoro-deoxyribonucleotides and/or 2′-O-methyl nucleotides.

21. The composition or method of claim 4, wherein one or more of the nucleotides of the inhibitory nucleic acid comprise one of both of ENA modified nucleotide or LNA nucleotides.

22. The composition or method of claim 4, wherein the nucleotides of the inhibitory nucleic acid comprise comprising phosphorothioate internucleotide linkages between at least two nucleotides or between all nucleotides.

23. The method or compositions of claim 4, wherein the inhibitory nucleic acid is a gapmer or a mixmer.

24. An inhibitor of Xist RNA and an inhibitor of an Xist-interacting protein, for use in activating an inactive X-linked allele in a cell, preferably a cell of a female heterozygous subject, and further preferably wherein the inactive X-linked allele is associated with an X-linked disorder.

25. An inhibitor of Xist RNA and an inhibitor of an Xist-interacting protein, for use in activating an epigenetically silenced or hypomorphic allele on the active X chromosome in a cell, preferably in a female heterozygous or male hemizygous subject, and further preferably wherein the active X-linked allele is associated with an X-linked disorder.

26. An inhibitor of Xist RNA and an inhibitor of an Xist-interacting protein, for use in treating an X-linked disorder in a female heterozygous or male hemizygous subject.


SEQUENCE LISTING (50 sequences)

SEQ ID NO: 1 - DNA, 15 bp, Artificial Sequence
  designed to not bind Xist
aacacgtctatacgc

SEQ ID NO: 2 - DNA, 16 bp, Artificial Sequence
  designed to target Xist
tcttggttactaacag

SEQ ID NO: 3 - DNA, 15 bp, Artificial Sequence
  designed to target Xist
agtagctcggtggat

SEQ ID NO: 4 - DNA, 16 bp, Artificial Sequence
  designed to target Xist
tgagtcttgaggagaa

SEQ ID NO: 5 - DNA, 16 bp, Artificial Sequence
  designed to target Xist
cggtgcctagcatgca

SEQ ID NO: 6 - DNA, 14 bp, Artificial Sequence
  designed to target Xist
gttgattgatagga

SEQ ID NO: 7 - DNA, 15 bp, Artificial Sequence
  designed to target Xist
gaaaggcacgaagag

SEQ ID NO: 8 - DNA, 14 bp, Artificial Sequence
  designed to target Xist
ggatgcttgaaaca

SEQ ID NO: 9 - DNA, 16 bp, Artificial Sequence
  designed to target Xist
tggaaaggaggaggtg

SEQ ID NO: 10 - DNA, 14 bp, Artificial Sequence
  designed to target Xist
gtgtatgcgtgtga

SEQ ID NO: 11 - DNA, 16 bp, Artificial Sequence
  designed to target Xist
ccttacccctctaaaa

SEQ ID NO: 12 - DNA, 16 bp, Artificial Sequence
  designed to target Xist
aggagcttaaagtgag

SEQ ID NO: 13 - DNA, 16 bp, Artificial Sequence
  designed to target Xist
ggttgaaaggaggaac

SEQ ID NO: 14 - DNA, 15 bp, Artificial Sequence
  designed to target Xist
ttcagcactacacat

SEQ ID NO: 15 - DNA, 15 bp, Artificial Sequence
  designed to target Xist
agatcaggatgtagc

SEQ ID NO: 16 - DNA, 15 bp, Artificial Sequence
  designed to target Xist
gcaggaaggaagctg

SEQ ID NO: 17 - DNA, 15 bp, Artificial Sequence
  designed to target Xist
cgatagatatgagac

SEQ ID NO: 18 - DNA, 15 bp, Artificial Sequence
  designed to target Xist
gatgtttctgctttg

SEQ ID NO: 19 - DNA, 15 bp, Artificial Sequence
  designed to target Xist
catccactcatcctt

SEQ ID NO: 20 - DNA, 15 bp, Artificial Sequence
  designed to target Xist
tcagtccctcactcc

SEQ ID NO: 21 - DNA, 15 bp, Artificial Sequence
  designed to target Xist
tctgtggttgttttc

SEQ ID NO: 22 - DNA, 15 bp, Artificial Sequence
  designed to target Xist
ctttctctcaattcc

SEQ ID NO: 23 - DNA, 21 bp, Artificial Sequence
  Synthetic primer
acggacaactgcgttgatttt

SEQ ID NO: 24 - DNA, 20 bp, Artificial Sequence
  Synthetic primer
acttagctgggaagcccaac

SEQ ID NO: 25 - DNA, 23 bp, Artificial Sequence
  Synthetic primer
atgaatacggctacagcaacagg

SEQ ID NO: 26 - DNA, 22 bp, Artificial Sequence
  Synthetic primer
ctcttgctcagtgtccttgctg

SEQ ID NO: 27 - DNA, 23 bp, Artificial Sequence
  Synthetic primer
cagagtagcgaggacttgaagag

SEQ ID NO: 28 - DNA, 22 bp, Artificial Sequence
  Synthetic primer
gctggttcgtctatcttgtggg

SEQ ID NO: 29 - DNA, 20 bp, Artificial Sequence
  Synthetic primer
tctaaggaagtcggggaagc

SEQ ID NO: 30 - DNA, 20 bp, Artificial Sequence
  Synthetic primer
ccctcgggtgtaatcagaat

SEQ ID NO: 31 - DNA, 22 bp, Artificial Sequence
  synthetic primer
gtgccatatcagtgagctctcg

SEQ ID NO: 32 - DNA, 21 bp, Artificial Sequence
  synthetic primer
aaccaaggttgagagagcaaa

SEQ ID NO: 33 - DNA, 23 bp, Artificial Sequence
  synthetic primer
tgtcacctaccaatgagagatcc

SEQ ID NO: 34 - DNA, 23 bp, Artificial Sequence
  synthetic primer
gcggtctggcagtaaaaactatc

SEQ ID NO: 35 - DNA, 23 bp, Artificial Sequence
  synthetic primer
gtgaaacagcattgctgtcactt

SEQ ID NO: 36 - DNA, 24 bp, Artificial Sequence
  synthetic primer
ctaggccacagaattgaaagatct

SEQ ID NO: 37 - DNA, 25 bp, Artificial Sequence
  synthetic primer
gtaggtggaaattctagcatcatcc

SEQ ID NO: 38 - DNA, 20 bp, Artificial Sequence
  synthetic primer
aaattgggttacaccgctga

SEQ ID NO: 39 - DNA, 20 bp, Artificial Sequence
  synthetic primer
ccacctagcctgcctgtact

SEQ ID NO: 40 - DNA, 21 bp, Artificial Sequence
  synthetic primer
ctgtatccttgggtcaagctg

SEQ ID NO: 41 - DNA, 20 bp, Artificial Sequence
  synthetic primer
ggagaagccattttccatca

SEQ ID NO: 42 - DNA, 20 bp, Artificial Sequence
  synthetic primer
acggaacgcagtaccaaaat

SEQ ID NO: 43 - DNA, 21 bp, Artificial Sequence
  synthetic primer
caaaaatccccaagaatgtga

SEQ ID NO: 44 - DNA, 20 bp, Artificial Sequence
  synthetic primer
cgttggctacccgtgatatt

SEQ ID NO: 45 - DNA, 22 bp, Artificial Sequence
  synthetic primer
tcagaagaactcgtcaagaagg

SEQ ID NO: 46 - DNA, 23 bp, Artificial Sequence
  synthetic primer
ggtaacaattttcccgccatgtg

SEQ ID NO: 47 - DNA, 23 bp, Artificial Sequence
  synthetic primer
ggaaataaacggaacgcagtacc

SEQ ID NO: 48 - DNA, 26 bp, Artificial Sequence
  synthetic primer
gactagacatgtcttaacatctgtcc

SEQ ID NO: 49 - DNA, 25 bp, Artificial Sequence
  synthetic primer
cctattgcatggacagcagcttatg

SEQ ID NO: 50 - DNA, 33273 bp, Homo sapiens
taacctctataggactctattttaatgtataaaacaggaataatcctttattattatctatgcaatacatattccattatctattacatgggataatggaatgggaagtcccttgaagatggtactaacctcaatgtattactcctttctagcttctttggttcaaaagtttggtggaggagttacaaattctggtttgaatgatatatttggatactttatcaacacatcaaagctctacctatcccttcccccattctcaaaaccaagctgaattaacatctttacatttattatgcagtttatggaggattttagcattaattattgcttgatttactcaatgtccccatgttatagatgagaactggaaaacccattgaagttgtgactcctggtctagaaatgaagtctacttccagttaatgttctttctggtatgtctttgctttcttgaaatttcccttttttgtccttactgggtaaattttgaaccaaccaaatcacaaagatgtccggctttcaatcttctaggccacgcctcttatgctctctccgccctcagcccccccttcagttcttaaagcgctgcaattcgctgctgcagccatatttcttactctctcggggctggaagcttcctgactgaagatctctctgcacttggggttctttctagaacattttctagtcccccaacaccctttatggcgtatttctttaaaaaaatcacctaaattccataaaatatttttttaaattctatactttctcctagtgtcttcttgacacgtcctccatatttttttaaagaaagtatttggaatattttgaggcaatttttaatatttaaggaatttttctttggaatcatttttggttgacatctctgttttttgtggatcagttttttactcttccactctcttttctatattttgcccatcggggctgcggatacctggttttattattttttctttgcccaacggggccgtggatacctgccttttaattcttttttattcgcccatcggggccgcggatacctgctttttatttttttttccttagcccatcggggtatcggatacctgctgattcccttcccctctgaacccccaacactctggcccatcggggtgacggatatctgctttttaaaaattttctttttttggcccatcggggcttcggatacctgctttttttttttttatttttccttgcccatcggggcctcggatacctgctttaatttttgtttttctggcccatcggggccgcggatacctgctttgatttttttttttcatcgcccatcggtgctttttatggatgaaaaaatgttggttttgtgggttgttgcactctctggaatatctacacttttttttgctgctgatcatttggtggtgtgtgagtgtacctaccgctttggcagagaatgactctgcagttaagctaagggcgtgttcagattgtggaggaaaagtggccgccattttagacttgccgcataactcggcttagggctagtcgtttgtgctaagttaaactagggaggcaagatggatgatagcaggtcaggcagaggaagtcatgtgcattgcatgagctaaacctatctgaatgaattgatttggggcttgttaggagctttgcgtgattgttgtatcgggaggcagtaagaatcatcttttatcagtacaagggactagttaaaaatggaaggttaggaaagactaaggtgcagggcttaaaatggcgattttgacattgcggcattgctcagcatggcgggctgtgctttgttaggttgtccaaaatggcggatccagttctgtcgcagtgttcaagtggcgggaaggccacatcatgatgggcgaggctttgttaagtggttagcatggtggtggacatgtgcggtcacacaggaaaagatggcggctgaaggtcttgccgcagtgtaaaacatggcgggcctctttgtctttgctgtgtgcttttcgtgttgggttttgccgcagggacaatatggcaggcgttgtcatatgtatatcatggcttttgtcacgtggacatcatggcgggcttgccgcattgttaaagatggcgggttttgccgcctagtgccacgcagagcgggagaaaaggtgggatggacagtgctggattgctgcataacccaaccaattagaaatgggggtggaattgatcacagccaattagagcagaagatggaattagactgatgacacactgtccagctactcagcgaagacctgggtgaattagcatggcacttcgcagctgtctttagccagtcaggagaaagaagtggaggggccacgtgtatgtctcccagtgggcggtacaccaggtgttttcaaggtcttttcaaggacatttagcctttccacctctgtcccctcttatttgtcccctcctgtccagtgctgcctcttgcagtgctggatatctggctgtgtggtctgaacctccctccattcctctgtattggtgcctcacctaaggctaagtatacctccccccccaccccccaacccccccaactccccacccccaccccccaccccccacctccccacccccctacccccctacccccctacccccctctggtctgccctgcactgcactgttgccatgggcagtgctccaggcctgcttggtgtggacatggtggtgagccgtggcaaggaccagaatggatcacagatgatcgttggccaacaggtggcagaagaggaattcctgccttcctcaagaggaacacctaccccttggctaatgctggggtcggattttgatttatatttatcttttggatgtcagtcatacagtctgattttgtggtttgctagtgtttgaatttaagtcttaagtgactattatagaaatgtattaagaggctttatttgtagaattcactttaattacatttaatgagtttttgttttgagttccttaaaattccttaaagtttttagcttctcattacaaattccttaacctttttttggcagtagatagtcaaagtcaaatcatttctaatgttttaaaaatgtgctggtcattttctttgaaattgacttaactattttcctttgaagagtctgtagcacagaaacagtaaaaaatttaacttcatgacctaatgtaaaaaagagtgtttgaaggtttacacaggtccaggccttgctttgttcccatccttgatgctgcactaattgactaatcacctacttatcagacaggaaacttgaattgctgtggtctggtgtcctctattcagacttattatattggagtatttcaatttttcgttgtatcctgcctgcctagcatccagttcctccccagccctgctcccagcaaacccctagtctagccccagccctactcccaccccgccccagccctgccccagccccagtcccctaaccccccagccctagccccagtcccagtcctagttcctcagtcccgcccagcttctctcgaaagtcactctaattttcattgattcagtgctcaaaataagttgtccattgcttatcctattatactgggatattccgtttacccttggcattgctgatcttcagtactgactccttgaccattttcagttaatgcatacaatcccatttgtctgtgatctcaggacaaagaatttccttactcggtacgttgaagttagggaatgtcaattgagagctttctatcagagcattattgcccacaatttgagttacttatcattttctcgatcccctgcccttaaaggagaaaccatttctctgtcattgcttctgtagtcacagtcccaattttgagtagtgatcttttcttgtgtactgtgttggccacctaaaactctttgcattgagtaaaattctaattgccaataatcctacccattggattagacagcactctgaaccccatttgcattcagcagggggtcgcagacaacccgtcttttgttggacagttaaaatgctcagtcccaattgtcatagctttgcctattaaacaaaggcaccctactgcgctttttgctgtgcttctggagaatcctgctgttcttggacaattaaagaacaaagtagtaattgctaattgtctcacccattaatcatgaagactaccagtcgcccttgcatttgccttgaggcagcgctgactacctgagatttaagagtttcttaaattattgagtaaaatcccaattatccatagttctgttagttacactatggcctttgcaaacatctttgcataacagcagtgggactgactcattcttagagccccttcccttggaatattaatggatacaatagtaattattcatggttctgcgtaacagagaagacccacttatgtgtatgcctttatcattgctcctagatagtgtgaactacctaccaccttgcattaatatgtaaaacactaattgcccatagtcccactcattagtctaggatgtcctctttgccattgctgctgagttctgactacccaagtttccttctcttaaacagttgatatgcataattgcatatattcatggttctgtgcaataaaaatggattctcaccccatcccaccttctgtgggatgttgctaacgagtgcagattattcaataacagctcttgaacagttaatttgcacagttgcaattgtccagagtcctgtccattagaaagggactctgtatcctatttgcacgctacaatgtgggctgatcacccaaggactcttcttgtgcattgatgttcataattgtatttgtccacgatcttgtgcactaacccttccactccctttgtattccagcaggggacccttactactcaagacctctgtactaggacagtttatgtgcacaatcctaattgattagaactgagtcttttatatcaaggtccctgcatcatctttgctttacatcaagagggtgctggttacctaatgcccctcctccagaaattattgatgtgcaaaatgcaatttccctatctgctgttagtctggggtctcatcccctcatattccttttgtcttacagcagggggtacttgggactgttaatgcgcataattgcaattatggtcttttccattaaattaagatcccaactgctcacaccctcttagcattacagtagagggtgctaatcacaaggacatttcttttgtactgttaatgtgctacttgcatttgtccctcttcctgtgcactaaagaccccactcacttccctagtgttcagcagtggatgacctctagtcaagacctttgcactaggatagttaatgtgaaccatggcaactgatcacaacaatgtctttcagatcagatccattttatcctccttgttttacagcaagggatattaattacctatgttacctttccctgggactatgaatgtgcaaaattccaatgttcatggtctctccctttaaacctatattctaccccttttacattatagaaagggatgctggaaacccagagtccttctcttgggactcttaatgtgtatttctaattatccatgactcttaatgtgcatattttcaattgcctaattgatttcaattgtctaagacatttcaaatgtctaattgattagaactgagtcttttatatcaagctaatatctagcttttatatcaagctaatatcttgacttctcagcatcatagaagggggtactgatttcctaaagtctttcttgaatttctattatgcaaaattgccctgaggccgggtgtggtggctcacacctgtaatcccagcactttgggaggctgaggtgggaagatcccttactgccaggagtttgagaccagcctggccaacattaaaaaaaaaaaaaagtaagacaattgccctggaatcccatccccctcacacctccttggcaaagcagcaggagtgctaactagctagtgcttcttctcttatactgcttaaatgcgcataattagcagtagttgatgtgcccctatgttagagtagaatcccgcttccttgctccatttgcattactgcaggagcttctaactagcctgaattcactctcttggactgttaatgtgcatacttatatttgctgctgtacttttttaccatgtaaggaccccacccactgtatttacatcccagctggaagtacctactacttaagacccttagactagtaaagttagcgtgcataatcttaggtgttatatacacattttcagttgcatacagttgtgccttttatcaggactcctgtacttatcaaagcagagagtgctaatcaatattaagcccttctcttcgaactgtagatggcatgtaattgcagttgtcaatggtccttcaattagacttgggtttctgacctatcacaccctctttgctttattgcatggggtactattcacttaaggcccctttctcaaactgttaatgtgcctaatgacaattacatcagtatccttccttttgaaggacagcatggttggtgacacctaaggccccatttcttggcctcccaatatgtgtgattgtatttgtcgaggttgctatgcactagagaaggaaagtgctcccctcatccccacttttcccttccagcaggaagtgcccaccccataagacccttttatttggagagtctaggtgcacaattgtaagtgaccacaagcatgcatcttggacatttatgtgcgtaatcgcacactgctcattccatgtgaataaggtcctactctccgaccccttttgcaatacagaagggttgctgataacgcagtccccttttcttggcatgttgtgtgtgattataatcgtctgggatcctatgcactagaaaaggagggtcctctccacatacctcagtctcacctttcccttccagcagggagtgcccactccataagactctcacatttggacagtcaaggtgcgtaattgttaagtgaacacaaccatgcaccttagacatggatttgcataactacacacagctcaacctatctgaataaaatcctactctcagaccccttttgcagtacagcaggggtgctgatcaccaaggccctttttcctggcctggtatgcgtgtgattatgtttgtcccggttcctgtgtattagacatggaagcctcccctgccacactccacccccaatcttcctttcccttccggcagggagtgccctctccataagacgcttacgtttggacaatcaaggtgcacagttgtaagtgaccacaggcatacaccttggacattaatgtgcataaccactttgcccattccatctgaataaggtcctactctcagaccccttttgcagtacagcaggggtgctgatcaccaaggccccttttcttggcctgttatgtgcgtgattatatttgtctgggttcctgtgtattagacaaggaagccttccccccgcccccacccccactcccagtcttcctttcccttccagcagggagtgccccctccataagatcattacatttggacaatcaaggtgcacaattataagtgaccacagccatgcaccttggacattattggacattaatgtgcgtaactgcacatggcccatcccatctgaataaggtcctactctcagatgccctttgcagtacagcaggggtactgaatcaccaaggccctttttcttggcctgttatgtgtgtgattatatttatcccagtttctgtgtaatagacatgaaagcctcccctgccacaccccacctccaatcttcctttcccttccaccagggagtgtccactccatatacccttacatttggacaatcaaggtgcacaattgtaagtgagcataggcactcaccttggacatgaatgtgcataactgcacatggcccatcccatctgaataaggtcctactctcagaccctttttgcagtacagcaggggtgctgatcaccaaggccccttttcctggcctgttatgtgtgtgattatatttgttccagttcctgtgtaatagacatggaagcctcccctgccacactccacccccaatcttcctttcccttctggcaggaagtacccgctccataagacccttacatttggacagtcaaggtgcacaattgtatgtgaccacaaccatgcaccttggacataaatgtgtgtaactgcacatggcccatcccatctgaataaggtcctactctcagaccccttttgcagtacagtaggtgtgctgataaccaaggcccctcttcctggcctgttaacgtatgtgattatatttgtctgggttccagtgtataagacatggaagcctcccctgccccaccccaccctcaatcttcctttcccttctggcagggagtgccagctccataagaaccttacatttggacagtcaaggtgcacaattctaagtgaccgcagccatgcaccttggtcaataatgtgtgtaactgcacacggcctatctcatctgaataaggccttactctcagaccccttttgcagtacagcaggggtgctgataaccaaggcccattttcctggcctgttatgtgtgtgattatatttgtccaggtttctgtgtactagacaaggaagcctcctctgccccatcccatctacgcataatctttcttttcctcccagcagggagtgctcactccataagacccttacatttggacaatcaaggtgcacaattgtaagtgaccacaaccatgcatcttggaaatttatgtgcataactgcacatggcttatcctatttgaataaagtcctactctcagaccccctttgcagtatagctggggtgctgatcactgaggcctctttgcttggcttgtctatattcttgtgtactagataagggcaccttctcatggactccctttgcttttcaacaaggagtacccactactttttaagattcttatatttgtccaaagtacatggttttaattgaccacaacaatgtcccttggacattaatgtatgtaatcaccacatggttcatcctaattaaacaaagttctaccttctcaccctccatttgcagtataccagggttgctgaccccctaagtccccttttcttggcttgttgacatgcataattgcatttatgttggttcttgtgccctagacaaggatgccccacctcttttcaatagtgggtgcccactccttatgatctttacatttgaacagttaatgtgaataattgcagttgtccacaaccctatcacttctaggaccattatacctcttttgcattactgtggggtatactgtttccctccaaggccccttctggtggactatcaacatataattgaaattttcttttgtctttgtcagtagattaaggtcataccccatcacctttcctttgtagtacaacagggtgtcctgatcaaccaaagtcctgttgttttggactgttaatatgtgcaattacatttgctcctgatctgtgcactagataaggatcctacctactttcttagtgtttttagcaggtagtgcccactactcaagactgtcacttggaatgttcatgtgcacaaactcaattctctaagcatgttcctgtaccacctttgctttagagcagggggatgatattcactaagtgccccttcttttggacttaatatgcattaatgcaattgtccacctcttcttttagactaagagttgatctccacatattccccttgcatcaggggcatgttaattatgaatgaacccttttcttttaatattaatgtcataattgtatttgtggacctgtgtaggagaaaaagaccctatgttcctcccattaccctttggattgctgctgagaagtgttaactactcataatctcagctcttggacaattaatagcattaataacaattatcaagggcactgatcattagataagactcctgcttcctcgttgcttacatcgggggtactgacccactaaggccccttgtactgttaatgtgaatatttgcaattatatatgtctccttctggtagagtgggatattatgccctagtatcccctttgcattactgcaggggctgctgactactcaaaacttctcctgggactgttaataggcacaatggcagttatcaatggttttctccctccctgaccttgttaagcaagcgccccaccccacccttagtttcccatggcataataaagtataagcattggagtattccatgcacttgtctatcaaacagtggtccatactcccaacccttttgcattgcgccagtgtgtaaaatcacaggtagccatggtgtcatgctttatatacgaagtcttccctctctctgccccttgtgtgcccttggcccctttttacagactattgctcacaatctcaggtgtccatatttgcagctattaggtaagattgtgctgtctccctcttcccttccctctgccctgccccttttgcctctttgctgggtaatgttgaccagacaaggccctttctcttggacttaaacaattctcagttgcactttccttggtcccacccattatacatgaacccctctacttcctttcgcattgcttctgagtatgctgactacccaaagccccttctgtgttattaataaacacagtactgattgtcccatttttcagcccatcagtccaagatctccctaccactttggtgtgttggtgcagtgttgactatgaaaagcaggcctgaactaggtggataagccttcactcattttctttcatttattaatgatcctagtttcaattattgtcagattctggggacaagaaccattcttgcccacctgtgttactgctttactgtgcaaaatactgaaggcaagtcagacccagggagctggattgccatcctttattttgtgtttccagtgtacactataaaattgtctccccaggaaggaaggttggcactttctctgcattcttctttccagagcagattgcctggttaagaatctcttgttgtcccctttgtatattgttattgtaaagtgccaaatgccaggatacagccagaaaaattgcttattattattaaaaaaatttttttaagaaagacatctggattgtagggtggactcgataacctggtcattatttttttgaagccaaaatatccatttatactatgtacctggtgaccagtgtctctcattttaactgagggtggtgggtctgtggatagaacactgactcttgctattttaatatcaaagatattctagagtggaactcttaagaccagtatctttgtgtgggctttaccagcattcacttttagaaaaactacctaaattttataatcctttaatttcttcatctggagcacctgcccctacttatttcaagaagattgcagtaaaacgattaaatgagggaacatatgcagaggtgcttttaaaaagcatatgccaccttttttattaattattatataaaatgaagcatttaattatagtaataatttgaagtagtttgaagtaccacactgaggtgaggacttaaaaatgataagacgagttccctattttataagaaaaataagccaaaattaaatattcttttggatataaatttcaacagtgagatagctgcctagtggaaatgaataatatcccagccactagtgtacagggtgttttgtggcacaggattatgtaatatggaactgctcaagcaaataactagtcatcacaacagcagttctttgtaataactgaaaaagaatattgtttctcggagaaggatgtcaaaagatcggcccagctcagggagcagtttgccctactagctcctcggacagctgtaaagaagagtctctggctctttagaatactgtaagtactacttcgtagctattaagtaatctttttcctattctattttctttctcttagatgccacctatagaaaagtcagagggtccagtaagtttctttccttcttcccacctcatctgcaatatatatatatagagagagaaatagatacatacatacatgcataaatacacatatgtgagttaaccagcagaactgtagaattaatattgtggacccagctctatgctaggttacactgataacctgggtaggaatgatatcatcctatataatttcattcctgagatgattttatcgttgaggagctaatgtgagcacatttgaaataactttagaaaataataagtgctgttttgtgtgaatcataagtagtagttttaggaagggaacccacaaggatttgaagttgatagaataaacttaaggaagtgggtttgctttttctctttaagccaagataggattaatattgcagccatctggatagtccagttggtttattttaatttcatttgttttttacctcttttggagccatggaaagagatgaaagggatagagcatagccattgtgtttggctatttgcgaaggttggcaaattagtgattgctaaatctcataagcttgagtattttaaagttcagagattgagggcataaatctaatacttcggctccttccacaattttactacatttctgcccaagaacagatgaccatggataatgcatatcgtagatactttttaagtttggaacctttttgccaagagggtagtggagaagtgaagtcaaaaccttgaccttccttgcctactttatgctgtagtttatataccttctttcctcccacctttcgtaaagctaaaagaagcttagcctccttaatgttttccagctgacaaaatattgtttaacataacattcgaaactttttttctggtgcacattcatgcatcacagcaggagcaacaagaaccatataagtgaactggcttcacttatagcccgttttaattcatatccatatttcctcagggcttgtttccatgcctcccagccccactccatatgcttaacaacattgtctggctgactgagggttatatacatcatggtcttgaaccttcttggaaacatggtctgtgccattgtttctcaaacccaagtaatgcttcatgatgaaacaccttctaaaggaacaaaattttctgagatcctaaaaaaatgtgttttgaggaacactgacttaacaaagatatttgaaatgtaaatatgttttccaatttcacgttgtctttgtcaaagatgtgttttatataacttatgtagaacttggggatccattagaatatattcacaaatccccagggttatcaccccaatttgagaaaccctggtctatgcttatgaaatcttctattggtaattaaattgtcattcattgtcaacatacaattataattattattggaatttgttttaaatgaatgaatttggaggtgattctgtaccttaagtcaagaggaaggatggcttgattttaggtggattgattatactagatagcatccaaaggtgaatcttgaagctgtatttaaattcattgcttgaaataatttccacccttaagaaaaatctctagcaattgtaaaaagggatgctctggaaatgtgggcatcttcaaaatagagataattcttgtgttagttcaacaaatattattgtaccaggtgctggaataaatagcaaaaccaaagacaggatttatatcaaggaatttgctttcttatggaggatgcagaaggaaatcattatggttttgggcagaaatgcttagactttagtcctggctctgagtttggttcagatcaccatcaatctgaccatctcgagactgctagtgaaataagataggggcttatatcaaatacctaaatccctgaaaatgacattttgtgatttggaaaattttcaaaagtctaatgaaggaaacttttttggcatttctttaaatgattattgtcatttcttttctgacttttccctttataaaaccttaacatgtaggattggaggaagttttctgaccattttctcatatcctctttcagctttatctttctgtaacttccatttctctagccacctccctaaattacagaagactgtgagacccagggctgctgtgattaggcattcataatttcttttcagggtgtttgtgccctgattatcaaatgtacagcttgaagggagttcatgtcttaaagtaatgaattaagagttgacctttgttgactgctaaaatattcttatatgtgaaagcatcctggaaaaatacgttaccagcttaaagagaaagaaactaatgattatatctgaactgagctaatgcctcttctcttcccccaaaccttatcagtttggatggcaaagagtaatgatgtgtcagttaaacagagctaatgccttcctctgccttgtcttaaagactggattgggagaaaattgatattctcactaccatattttgggctgtaggcaagtagcattttacacaggtttccttcaaaaatccaactcaagttggagctcatgtatttaagacatagctggcctgctgaatttaacaagttaaacttcagtggccatgtacagttatatatcactatatatatgtgtattaggctgtcgagttggtcatgtttttgttggtgacttaggctttacttgatagctcttccttgacctttccaaattgagtactgatacatggagcttgggcttcttctgcatcttatacaaatgagtttggtaaagaagcctctcctttactgttttgatgtttatattagaaataacttttgattattttttttcatgttaggatgagaaactgaaacaaaatgtaaatttgaccggtgctagacttcttaaattatgggtagacttaaagtattattttccttaaccaattagaatgctagtcttctagtgttcccggaaacatgagaggttatgcagtagacccaagcaataccctcttattacataatcaagtgcgtataagaatttaaaaatagggatatgactggaacatcactgtactttaccaggtcccattataaaattatctatgttactttacccatagctttgaaaactagtggcatagtatattttatagtatgctgttagtgtgattggcattgaacagtgatgggatataatcactctacaatctatatgttattaaagttttccagccttatagatctcccttgactgaaaattagctactaacttacgacttattttttacagcagattgactaggtctttccaggaaatctgttgatgtacaaaaacaaagtttaattgctaatgtttttttaaaaaataactttttgatattacggatacctggttatttgggccttgtatattttaacatcaaaattacctattataaatccatataaacagaaaagaaagagagtaagtctttagatcagatctgcaaacaatgatggtacgtactgtagaaaaatctggaacatagacttaccagttcttaggttccattttgcttgctttttaaaaactgtgtcttataagtcttcagcaactggttgggagatttttagaaaaaataaccttttaatgttagaacagtgtagagatttacagaatgattctgaagatagagtttctgtgtacttcacacccagtttttcccagtgttaacattttacattagtttggtacatttgtcacaacaaaccaatattgatacattattattaactagagtccatattttattcagatttccttagtttttccttaatgttctttttgtgttccaggatcccattgaagataccacgctgcatgtgtccttagtagtcatgtctccttaggctcctcttggtaatgacagtttctcagactctttgtttttgatgaacttcacagttttgaggactaatggtccagtattctatagaatgtctctctattggaatttgtctgatgttcttctcatgactagattgggtttatgagtgtttaggaggaagaccacaaaggtagagtgccattcttatcacttatcaagagtacatactatcaacatgacttatcactgtttatgttatccttaatcacctgtctgaggtactatttgtcaggtttctccagcgtaaaattagtctttatttctccatttccctactatactgttcacataggaagtcactatgtgcagccagcacttaaggaatgggaaattaccttccacctcattgagggcagagtatttacataaattatttggaattcttttgcacaggatgtcttttctccacaatgtattgtgtttattcagtcatttatatcagtatgatctcagggatattttatactctgggttataatacagtattactttattctgttgttcaaattgttccagctttggccattgggaggtctttcatttggctttgatataaccccatgaatgtgggttttttgtttgagcactttcttatttttggaactacaacatgcttcagactcatttgcatatctcctgcctggacctaaaatgatgtatttctgcaaggagccttgatactttttattggagagtaatattagaaatcaagaagtgaatgctaggtgcgctcattactactggagtgtcattccttcaagaccttttcagttgacaagagcaaggagatatatatttgcattctaacgtgtgtatatgcacatagctataaatatatataaccatctgtatctatattaaactaaatgtgtttatacctacgtctccaactctaatcattgccacatggatcattatagtctcacctccttgcttatctgttacctcccatttctacagtgagaaacctggcttggttgggaaatttttctgttaatattacggtagtgagtgtttgacatttgcttctatggttaagtttagggagagtttagctgtagggtattcttgaaactagaaatgacccttctgccctaaatgtttctgccagttttgaaacgtaaaataggttgcagaaacaaactttatcttaagaaccagaatttacttcaatccacattttgacattgattttcagattaaattattctgatatcgccaggtaagctgttccttgggtatgcatttcttctttccgtttttttctaagagctaaaggaccctgagaacactggaggtgggaaaggaagggaaaggcatgttcacacgtgggataggaaaggttcatttactgacctccagctagccttccaaagtgcctatttaagacccaaggagtagatgtcttccttggcaattgtaacccaaatataatttttaacctttcaattttagtcaagaaagttggtgtgctgttacaaaaagtgccctgattaacagcattgtcatgtgcattgcatattaatcagcaatttaaaataacatgaaattatgttgagtataattttaatattttatattagatattagtttgagacagtgtttctcaagtctgtataataagtttgatagtagggaggttttctctcaagaaaagaattattcagtgtgcacctacataatcactgcttagattctacaattaatattttgctatatttgattaaacgttttctgtaaaagaaaaatattattatgtactatttaggtttatgggaataattgttaagttaaagtgtatgaacaaacctggaatgaaatctgtttgcctacatctataatacaactataaaacatagcagatgtacaaattagtagttaatagataactaaaatgcaaatatggcactactattatagtattatagtttcttttgagtggcgtgtctgtaatatcacatgctgtgttgatgcacttcaccaaactgctgttttcaaactgctttaaatcctgccattatagcacatagcaatgctatttcactttcatttggcacaaaacacatttatatattgtttgcttctcttcttttctgtaatccccaggcaacaaaactagaacatttgccactaatctggcaacgtggtcctatattatgaagtagtcatatagctgatctaaactatccttacagtgaaatgagagtattgtgaaagttttgtagaaagctccccatatgtcctgagaatctatgcacagaccccacagttaaaagacctttgaattgtgggaagacatgggtttaagtatcacttggttaccttctatttgtgtaacattgaggtagtttcatcttctgggttcccagtttccttagagaatgaaaatgttgaattatgtgatttttttttttttttgagacggagttttgctctttcgcccaggctggagtgaagtagcacgatctcgactcactgcaacctccttcccccatgatcaagcaattctcctgcctcagcctcccaagtagctgggattacaggcacccgccccccaccccccgcccccagctaatgtttgtatttttagtacagatggagttttgccgtgttggccaggctggtctcgaacttctgacctcaggtgatccactcgccttggcctcccaaagtgctaggattacaggcatgagccactgcgcctggcctatgtgattattaatatcacgtctagctgtgacaattctgtctgatgctggagtatttgaaccagatggctggctgtgccactcagttattctctccataagactttgatattttgttggtctgcaagatgacggattctcaaaattcttgtcagtgaatattgaaccctagtgaaatgtatggttctgtatcagttccaaaatgtaaccactttctctagccttagattcccagttccaaaatgtaaccattttctctagccttagattcccgttaagggaaagggaatgctctttgagtatgtcatcaccatagtaacaggcaaaactagagggctttgatgctaaagcaagatactccataaatatgcttaagaagacttggggagactggaatagttgttcccttttagatgccagtgtataaatgaatttgagctaggatccgtttatttaaaatttctttaggtgtatttgcttgcatatggagtgcacatttactctcattaatggagttttaggaagcagtagagtaaatgcataaacatgtatgaaccgccatgtttaactggaagcctgcatttggaagtcaagtatctaatcttagattaaattaggatggggaaggatgttggcaagagattttgaagcttgttctgcttatattgagaacatcatagaacagtttggcctttttaaagctagagaatagtgttgaataagtgatgttccatatattcctgtttgacattgacataaaggtttcctcatgatacagtaatccctgatcagggatctggaagcctgtattcatttaaggtactcaggtttaacatactgggtgcttttcacaccatactatacagtaccatgcaaagtgctttcaagactgcaaatttggcttagatcccctttagtgagctcctatgctatagtaaaggtagatagccaattattaaaaacagtcaagacaattgcacctctaagcagtagtagcagttgccacaccaccttgaatcttgaagtattttcagcaacaggatgaccattagccacaaatttagtgtcagcccttaaggtcggtattggtttgacccatattttcatgtagttctttttcttcacttgtctaatcttcccgtgtactgccagggcttgtcattagaggactttagggagaccaagcaggctagaaagtagagacaggagatacctatgtctaatgcttcagtttatacttcctaggtttttttcattggggtttttgtaactcttttggtatcctaccggtgctttggtagcctactgaaccctgtctttcttcttaaggacattctgagcatgtgagacctgaggactgcaaacagctataagaggctccaaattaatcatatctttccctttgagaatctggccaagctccagctaatctacttggatgggttgccagctatctggagaaaaaggtagtttggggaatttattgttgtagtgcttctgtctttggattgaacttcccacaactctcctttttaaagcagaacacagctgggcatggtggctcctgcttgtaattccagggctttgggaggttgaggtggggggatcacttgaggccaggagttgaagacccatgtctctacaataaaataaaattagttgggcatggtggtacgtgcctgtagtcctacctactctggaggctgaggcagcaggattgcttgagcccaggagttcaaggctgcagtgagccatcattagccactgcactccagcctaggtggcagagcgggacccagtctcttaaaaagaaagaaaagcagaacgtgagccagttttcatcaattcctatactttttcttttgcatgtacacatacattttaactttacataatgagttcggcctgtttcatttatccctcagagctgggctccagtgaggtctgtaagggcaagcatacttgatccccaatgaagaatgagagatgcaaagcactaaattatttcttttctcaccacacagcaagatagatttaatgaacttaacaccttttgattagtggccttttaaattattcccactttcctttggcagatgggtattaagttctcaggatttgtttacaaataagactaacttcatctgtattagctcagttttggtaggcctaattccattatcactgccatttccttgttttaagaaatcaaaatttcttagcttgaaaaacaattgaaattgttaaaaagtggaataggagagccccgggggcctgtataaggaatttactgaatccctggttttctgtaccttgtttttccttctgcatagatttgcttaactgtttttgtggcgtgtattttttttttttcgcagtttcgctcttgttgcccaggctggagtgcaatggcgcaatctcagctcactgcaacctctgtctcctgggttcaagttattctcctgcctcagcctctcgagtagctgagattacaggcatgcgcgaccacgccaggctaattttgtatttttagtagagacggggtttctccatgttggtcaggctggtctcaaactcctgacctcaggtgattcacccgcctcgacctcccaaactgctgggattacaggcgtgagccaccacgcctggccagctgttgttataactggagttctatgtgcttgtgaccattcttggtttctccgaatatcctagaactttggtggcgccctattatacaggttgttgaagaaatgttaccatgtggattgagtaggaaacaattctctttatcttggcaatattatggcatggcactacttaaagtacaaattaaaagagggggatgctacagaactagctgacaggcactttgatagaggtggatttctcagttcttaaaatagctctttataaaggaagccagaggcattgtggaggagaattcttacataactcatagggttagaccacatccgaccttttctgtgtggcttcatggctctcttggttgagaaagcattagtttctccttccattagtttcaacctcttgatttcttgacccccctactatattttgtgctgagaacacaagggtattaacaacccacattgtagaggatcgctcagtaataaagactggagaataaaatgcagcatgggaatattggcaattactcagttctaaatttctcttggaaatgagggaaagcatacagaatagagctggaatgaataggataattttttttttttttgctaagttggtagccagaatataacagctccgcacaactgtaaatgtccactcttcaatccacatgaagaaaagggtaaaaatatggttgaactcaaccactagttgcccattagaacagactttcccagtgtactgcatttcaatactttttcttttatctcttttcagatcttcctcagaagaataggcttgttgttttacagtgttagtgatccattccctttgacgatccctaggtggagatggggcatgaggatcctccaggggaaaagctcactaccactgggcaacaaccctaggtcaggaggttctgtcaagatactttcctggtcccagataggaagataaagtctcaaaaacaaccaccacacgtcaaggtgcgtaagctgtccctaaaagcataataagtagtcttaattttgattttgttttccagtatacattgcacttagtgtttcactgaggtcgtattcatcattattctgcatatgatttggtaaaaacagcttcctaactaacctgggaagcaactgggtgtgagattaactggttaaagtgatgatgtaaagagggtagcgggttgcatgtgttcgggtgtttggagtgggactatagcacgtggcagaggcttacagctaagttgttcttttaggagaacatggacaactgtcacatcagtgacattgatcacatgggcaaatcattctgttccatgtggtccccaaagtctctcttaaagccttacagaagaactttgccaatcatttacatacttcaggatggcttgggatgccatggtgtataatacaacaagtgagaggtgtgtctttttatgctatggttgctgattgatggaagccgcataaatacaaatggaaacctgactaaaaatggcacaaagttatctgtcatcaggcaggagctaaagaaccaggaccctacattctctaggtcagtgttgggagaggctgattagcgagtgagaattggcagataaaggtgaccattcggtgcaataaatcctgaacgtataggctttgcccagcattcttcgtaaatagtgggtagctataaatttcatgaaatattttcatgggtaagaactcttgaaatgttataattgactagaaatctctgtagatttagaaatagagagttactaacaaattgttagaaagtctaggaactagaaagctaagttgagagttatctaggaagatctatctattgtactcataatctttagataaattctcctagggccagtagtctatgtgaattttctttttcttcttcttcttcttctttttttttgtattttagctgcaatgttaaacaacctatgtgaattttcttattgtgagaatatttgccttccagagtgactcacctttatctcaaagagcaatattgtgagttttgaaaatgctgctctaaggctgtgttttgttagtcctgagccaggagacttaaagcaaacttgaggggtcttaaaacatcgaagtgagccttaaacattgggaagaccttatgtttttccctctcatatctattatttttgtgatctcagttattaatcatttaaagggactctttcctagctgattggcacttaaaacaggatggaagtctttttttttttttttttttttgagatggagttttgctcttgttgcccaggctggagtgcaatggtgcaatctcagctcactgcaacctctgcctcccgggttcaagcgattctcctgcctcagcctcccaagtagctgggattacagtcatgcaccaccacgcccggctaattttgtatttttaatagagacggtgtttctccatgttggtcaggctggtctcaaactcctgacctcaggtgatccgcccacctcaacctcccaaagtgctgggattatgggcgtgagccaccgcgcccggcagttctggtctttaactaaggtataaggctatgactggtagtggtgtctctagtgactcatcaagtgatatttggcaagacattttcccatttatgccagtttcctattctgttgaatgaggaaattttctctctaaagacctaaaagttttgactttataggtttcaaagttctgtggaaacattttctattgcttattaatttgaatcttatgtaactctagcacagtactcaatatttatggcatttacatggtttatctcatgtttttttatagctcttcattgttcctatctgccaaatcattatacttcctacaagcagtgcagagagctgagtcttcagcaggtccaagaaatttgaacacactgaaggaagtcagccttcccacctgaagatcaacatgcctggcactctagcacttgaggatagctgaatgaagtaagttgttgatgttgcagtcctgtgaggatcacttcagaactgttataacagctgttttttgggagctggtgttggatggggtgtgttggtctaatgtgaagtggggctaaatgtgagatggaaagatgaccagtcttccatattactgactgggttcactgaagcaactcaaagacattatggtcttcttaccagttgtatcacagaagaatttagcctttgcttgtgtgttctatgtcttcactgtataggccctctgtcattcttagagccttaaacgttgagaagcttaaaacaccatttctgctttctgctgaaagggtaaccctttctcatctccgtttgtgagagactctgtcgtcagttaagattagtgtaaaaagaaaactaaactctgaagtagccattataaaagtgtgagaatgaagtcagttttctaaagagttggggaaaggtgatgctaaaggaggggattgagcaagtcctatcaaagagccttttatgaaaatacttagtcatctgtgacatcccatttggctcttccagaaatcctagtaaatagttgtaacaggatgttaagaggcatacattgtgtgttttaaatcctctgctactcattaggtatatgacctttgacaacttaaagtctctagacttctctgtttgtgagggttaaatgaaatcatgtatgtaaagtgctcacctattgcagtgcctggcacatgtcaagtaaaaggtaacccaagaagactcataagttcatttcccacaatataagtgaccactagcactatcaggtagcaggcagagttggcatgctttggttctatgtaagaaatccctaaggtaaaagtttataaatagaagagcatctgtgttggtattggtggttgttattattgtagtactataagtagtattcgtagtaacaatagtttattataattactaatgacactttttgattttttttatctttctgtgatgcttttcatgcctcttgtgcccctcactgtatcttgcctcttctactacttacttcctctgaatgtctgcctttgcttatctcttgcactcaagtgtgtatttctttgtctctttctttcttgtctttgctctttgttctctatctaaagtgtgtcttacccatttccatgtttctcttgctaatttctttcgtgtgtgcctttgcctcattttctctttttgttcacaagagtggtctgtgtcttgtcttagacatatctctcatttttcattttgttgctatttctctttgctctcctagatgtggctcttctttcacgctttatttcatgtctcctttttgggtcacatgctgtgtgctttttgtccttttcttgttctgtctacctctcctttctctgcctacctctcttttctctttgtgaactgtgattatttgttaccccttccccttctcgttcgttttaaatttcaccttttttctgagtctggcctcctttctgctgtttctactttttatctcacatttctcatttctgcatttcctttctgcctctcttgggctattctctctctcctcccctgcgtgcctcagcatctcttgctgtttgtgattttctatttcagtattaatctctgttggcttgtatttgttctctgcttcttccctttctactcacctttgagtatttcagcctcttcatgaatctatctccctctctttgatttcatgtaatctctccttaaatatttctttgcatatgtgggcaagtgtacgtgtgtgtgtgtcatgtgtggcagaggggcttcctaacccctgcctgataggtgcagaacgtcggctatcagagcaagcattgtggagcggttccttatgccaggctgccatgtgagatgatccaagaccaaaacaaggccctagactgcagtaaaacccagaactcaagtagggcagaaggtggaaggctcatatggatagaaggcccaaagtataagacagatggtttgagacttgagacccgaggactaagatggaaagcccatgttccaagatagatagaagcctcaggcctgaaaccaacaaaagcctcaagagccaagaaaacagagggtggcctgaattggaccgaaggcctgagttggatggaagtctcaaggcttgagttagaagtcttaagacctgggacaggacacatggaaggcctaagaactgagacttgtgacacaaggccaacgacctaagattagcccagggttgtagctggaagacctacaacccaaggatggaaggcccctgtcacaaagcctacctagatggatagaggacccaagcgaaaaaggtatctcaagactaacggccggaatctggaggcccatgacccagaacccaggaaggatagaagcttgaagacctggggaaatcccaagatgagaaccctaaaccctacctcttttctattgtttacacttcttactcttagatatttccagttctcctgtttatctttaagcctgattcttttgagatgtactttttgatgttgccggttacctttagattgacagtattatgcctgggccagtcttgagccagctttaaatcacagcttttacctatttgttaggctatagtgttttgtaaacttctgtttctattcacatcttctccacttgagagagacaccaaaatccagtcagtatctaatctggcttttgttaacttccctcaggagcagacattcatataggtgatactgtatttcagtcctttcttttgaccccagaagccctagactgagaagataaaatggtcaggttgttggggaaaaaaaagtgccaggctctctagagaaaaatgtgaagagatgctccaggccaatgagaagaattagacaagaaatacacagatgtgccagacttctgagaagcacctgccagcaacagcttccttctttgagcttaggtgagcaggattctggggtttgggatttctagtgatggttatggaaagggtgactgtgcctgggacaaagcgaggtcccaaggggacagcctgaactccctgctcatagtagtggccaaataatttggtggactgtgccaacgctactcctgggtttaatacccatctctaggcttaaagatgagagaacctgggactgttgagcatgtttaatactttccttgatttttttcttcctgtttatgtgggaagttgatttaaatgactgataatgtgtatgaaagcactgtaaaacataagagaaaaaccaattagtgtattggcaatcatgcagttaacatttgaaagtgcagtgtaaattgtgaagcattatgtaaatcaggggtccacagtttttctgtaaggggtcaaatcataaatactttagactgtgggccatatggtttctgttacatatttgttttttaaacaacgtttttataaggtcaaaatcattcttagtttttgagccaattggatttggcctgctgttcatagcttaccaccccctgatgtattatttgttattcagagaaaatttctgaatactactagtttccttttctgtgcctgtccctgtgctaggcactaaaaatgcaatgattattgatatctaggtgacctgaaaaaaaatagtgaatgtgctttgtaaactgtaaagcacttgtattctactgtgataagcgttgtggatacaaagaaaggagcaagcataaaaaagtgctctttcaaaaggatatagtactatgcagacacaaggaattgtttgataaatgaataaattatatgtatatttgaggccaatttgtgtttgctgctctggtaattttgagtaaaaatgcagtattccaggtatcagaaacgaaaacacatggaaactgcttttaaactttaaaatatactgaaaacataagggactaagcttgttgtggtcacctataatgtgccagataccatgctgggtgctagagctaccaaagggggaaaagtattctcatagaacaaaaaatttcagaaaggtgcatattaaagtgctttgtaaactaaagcatgatacaaatgtcaatgggctacatatttatgaatgaatgaatggatgaatgaatattaagtgcctcttacataccagctattttgggtactgtaaaatacaagattaattctcctatgtaataagaggaaagtttatcctctatactattcagatgtaaggaatgatatattgcttaattttaaacaatcaagactttactggtgaggttaagttaaattattactgatacatttttccaggtaaccaggaaagagctagtatgaggaaatgaagtaatagatgtgagatccagaccgaaagtcacttaattcagcttgcgaatgtgctttctaaattataaagcacttgtaaatgaaaaatttgatgctttctgtatgaataaaactttctgtaagctaggtattgtctctacaaaattctcattgtatagttaaaccacagtgagaagggttctataagtagttatacaaaccaagggtttaaatacctgttaaatagatcaattttgattgcctactatgtgaactcactgttaaaggcactgaaaatttatcatatttcatttagccacagccaaaaataaggcaatacctatgttagcattttgtgaactctaaggcaccatataaatgtaactgttgattttctcacttggtgctgggtactaggtttataaaattgtatgatagttattatattgtgcaaataaagtaggaaaatttgaataacaatgattatcttttgaatacgcatacgcaagggattggttgtctgaagaatgccactatagtagttatctattgtgtgccaatctcattgctaggcattggggatgcaaagataaaccatctttattgtgtcttgggtagcagaagaaaatatgtgtaaaatcaatttataatttgtaaactgccacccatatataagctatatctgctgaatgatcattgattactcttatccttagagataacaactgggggcacaaacatttattatcattattgaacctacaacagagatctatgtgtagatttacaaagcctacagttctatacagataggaatgaactattggcttactgaatggtgattactttctgtggggctcggaactacatgccctaggatataaaaatgatgttatcattatagagtgctcacagaaggaaatgaagtaatataggtgtgagatccagaccaaaagtcatttaacaagtttattcagtgatgaaaacatgggacaaatggactaatataaggcagtgtactaagctgagtagagagataaagtcctgtccagaagatacatgcttcctggcctgattgaggagatggaaaatttttgcaaaaaacaaggtgttgtggtcttccatccagtttcttaagtgctgatgataaaagtgaattagacccaccttgacctggcctacagaagtaaaggagtaaaaataaatgcctcaggcgtgctttttgattcatttgataaacaaagcatcttttatgtggaatataccattctgggtcctgaggataagagagatgagggcattagatcactgacagctgaagatagaagaacatctttggtttgattgtttaaataatatttcaatgcctattctctgcaaggtactatgtttcgtaaattaaataggtctggcccagaagacccactcaattgcctttgagattaaaaaaaaaaaaaaaaagaaagaaaaatgcaagtttctttcaaaataaagagacatttttcctagtttcaggaatcccccaaatcacttcctcattggcttagtttaaagccaggagactgataaaagggctcagggtttgttctttaattcattaactaaacattctgcttttattacagttaaatggttcaagatgtaacaactagttttaaaggtatttgctcattggtctggcttagagacaggaagacatatgagcaataaaaaaaagattcttttgcatttaccaatttagtaaaaatttattaaaactgaataaagtgctgttcttaagtgcttgaaagacgtaaaccaaagtgcactttatctcatttatcttatggtggaaacacaggaacaaattctctaagagactgtgtttctttagttgagaagaaacttcattgagtagctgtgatatgttcgatactaaggaaaaactaaacagatcacctttgacatgcgttgtagagtgggaataagagagggctttttattttttcgttcatacgagtattgatgaagatgatactaaatgctaaatgaaatatatctgctccaaaaggcatttattctgacttggagatgcaacaaaaacacaaaaatggaatgaagtgatactcttcatcaaacagaagtgactgttatctcaaccattttgttaaatcctaaacagaaaacaaaaaaaatcatgacgaaaagacacttgcttattaattggcttggaaagtagaatataggagaaaggttactgtttattttttttcatgtattcattcattctacaaatatattcgggtgccaataggtacttggtataaggtttttggccccagagacatgggaaaaaaatgcatgccttcccagagaatgcctaatactttccttttggcttgttttcttgttaggggcatggcttagtccctaaataacattgtgtggtttaattcctactccgtatctcttctaccactctggccactacgataagcaggtagctgggttttgtagtgagcttgctccttaagttacaggaactctccttataatagacacttcattttcctagtccatccctcatgaaaaatgactgaccactgctgggcagcaggagggatgatgaccaactaattcccaaaccccagtctcattggtaccagccttggggaaccacctacacttgagccacaattggttttgaagtgcatttacaaggtttgtctattttcagttctttactttttacatgctgacacatacatacactgcctaaatagatctctttcagaaacaatcctcagataacgcatagcaaaatggagatggagacatgatttctcatgcaacagcttctctaattataccttagaaatgttctcctttttatcatcaaatctgctcaagaagggctttttatagtagaataatatcagtggatgaaaacagcttaacattttaccatgcttaagttttaagaataaaataaaaattggaaataattggccaaaattgaaaggaaaaatttttttaaaatttctctaaatgtaggcctggctgggctttgaccttttccgtttttaaatcactcacagagggtgggacaggaggaagagtgaaggaaaaggtcaaacctgttttaagggcaacctgcctttgttctgaattggtcttaagaacattaccagctccaggtttaaattgttcagtttcatgcagttccaatagctgatcattgttgagatgaggacaaaatcctttgtcctcactagtttgctttacatttttgaaaagtattatttttgtccaagtgcttatcaactaaaccttgtgttaggtaagaatggaatttattaagtgaatcagtgtgacccttcttgtcataagattatcttaaagctgaagccaaaatatgcttcaaaagaagaggactttattgttcattgtagttcatacattcaaagcatctgaactgtagtttctatagcaagccaattacatccataagtggagaaggaaatagataaatgtcaaagtatgattggtggagggagcaaggttgaagataatctggggttgaaattttctagttttcattctgtacatttttagttagacatcagatttgaaatattaatgtttacctttcaatgtgtggtatcagctggactcagtaacacccctttcttcagctggggatggggaatggattattggaaaatggaaagaagaaagtaactaaaagccttcctttcacagtttctggcatcactaccactactgattaaacaagaataagagaacattttatcatcatctgctttattcacataaatgaagttgtgatgaataaatctgcttttatgcagacacaaggaattaagtggcttcgtcattgtccttctacctcaaagataatttattccaaaagctaagataaatggaagactcttgaacttgtgaactgatgtgaaatgcagaatctcttttgagtctttgctgtttggaagattgaaaaatattgttcagcatgggtgaccaccagaaagtaatcttaagccatctagatgtcacaattgaaacaaactggggagttggttgctattgtaaaataaaatatactgttttgaaaactttgtattttgatgtgacaatttctaactcactgtccctaaccacactaattaccgtaagttcttcatttcttttttctttttctttttaaattaataaattaatttattttttggagatggagtctcgctttgttgcccaggctggagtgcagtggtgccatcttggctcactgtaacctctgcctcctgggttcaagcgattctcctgcctcagcctcaccagtagctgggattacagtcatgagccaccatgcctggctaatggttgtatttttagtagagacggggttttgccatgttggccaggctggtcacgaactcctgacttaaagtgaaacacctgcctcggcctcccaaagttctgggattacaggcgggagccaccgtgcctggcctcatttctttttttaaactaccttaatcacccagtgtgggtaaatggtttgaagtctcatttcttacagcatcagttgctttggtcaaggatactgagaagcacaaacccagtaaaaaatgtgagaagcaaaaaacttgaggatcaaaagtgggagaggagaagggtataaactgtaaggctgtgggaattaagcattcatgaaaga